(19) United States
(12) Patent Application Publication
(10) Pub. No.: US 2005/0282819 A1
(43) Pub. Date: Dec. 22, 2005

(54) Heterocyclic substituted indane derivatives and related compounds for the treatment of schizophrenia

(76) Inventors: James M. Graham, Ann Arbor, MI (US); Brian Edward Kornberg, Ann Arbor, MI (US); Sham Shridhar Nikam, Ann Arbor, MI (US); Dejian Xie, Glenmont, NY (US)

Correspondence Address: WARNER-LAMBERT COMPANY, 2800 PLYMOUTH RD, ANN ARBOR, MI, 48105, US

(21) Appl. No.: 11/007,486

(22) Filed: Dec. 8, 2004

Related U.S. Application Data
(60) Provisional application No. 60/527,852, filed on Dec. 8, 2003.
(60) Provisional application No. 60/531,096, filed on Dec. 19, 2003.

(51) Int. Cl. A61K 31/496; C07D 417/02

(52) U.S. Cl. 514/254.04; 544/368

(57) ABSTRACT

This invention relates to compounds of the formula 1 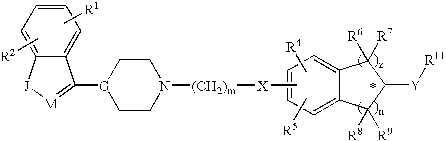
wherein J, M, G, m, X, R¹, R², R⁴, R⁵, R⁶, R⁷, R⁸, R⁹, Y, n, z, and R¹¹ are defined as in the specification, pharmaceutical compositions containing them and their use in the treatment of central nervous system and other disorders.

This application is a United States Utility application, which claims the benefit of priority to the U.S. Provisional Application Ser. No. 60/527,852, filed Dec. 8, 2003 and U.S. Provisional Application Ser. No. 60/531,096 filed Dec. 19, 2003.

### BACKGROUND OF THE INVENTION

This invention relates to heterocyclic substituted piperazines, pharmaceutical compositions containing them and their use for the m treatment of schizophrenia and other central nervous system (CNS) disorders or conditions.

The heterocyclic substituted piperazine derivatives of this invention exhibit activity as antagonists of dopamine D2 receptors and of serotonin 2A (5HT2A) receptors.

Other heterocyclic piperazine derivatives that are useful for the treatment of schizophrenia are referred to in U.S. Pat. No. 5,350,747, which issued on Sep. 27, 1994, and in U.S. Pat. No. 6,127,357, which issued on Oct. 3, 2000. These patents are incorporated herein by reference in their entireties.

Other piperazine and piperidine derivatives that have been stated to be useful as antipsychotic agents are those referred to in PCT patent publication WO 93/04684, which published on Mar. 18, 1993, and European patent application EP 402644A, which was published on Dec. 19, 1990. These patent applications are incorporated herein by reference in their entireties.

### SUMMARY OF THE INVENTION

The present invention relates to compounds of the formula 1

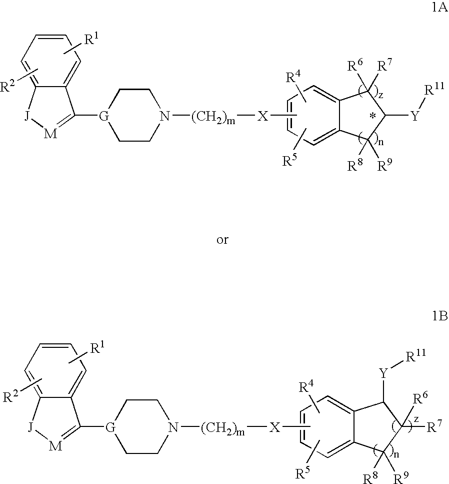

wherein J is S, SO, SO₂, CH₂, O, or NR¹⁰ wherein R¹⁰ is hydrogen, (C₁-C₆)alkyl, C(═O)(C₁-C₆)alkyl, or C(═O)O—(C₁-C₆)alkyl;

M is CH or N;

G is CH or N;

m is an integer from one to six;

X is O or NR³ wherein R³ is defined as R¹⁰ is defined above, C(═O), CHOH, CHOR³, CH(halo), or CHNR³R¹², wherein R¹² is defined as R¹⁰ is defined above;

or X is absent;

R¹ is hydrogen, halogen, cyano, (C₁-C₆)alkyl optionally substituted with from one to three fluorine atoms, (C₁-C₆)alkoxy optionally substituted with from one to three fluorine atoms, or R¹ forms a heterocyclic ring with R¹⁰;

R² is defined as R¹ with the proviso that R² cannot form a heterocyclic ring when R¹ is present;

R⁴ and R⁵ are, independently, hydrogen, halogen, cyano, amino-(C₁-C₆)alkyl, (C₁-C₆)alkylamino-(C₁-C₆)alkyl, di(C₁-C₆)alkylamino-(C₁-C₆)alkyl, hydroxy(C₁-C₆)alkyl, (C₁-C₆)alkoxy, or (C₁-C₆)alkoxyalkyl, wherein each of the alkoxy and alkyl moieties of the foregoing R⁴ and R⁵ groups can be optionally substituted with from one to three halo atoms, preferably with from one to three fluorine atoms;

R⁶, R⁷, R⁸, and R⁹ are, independently, hydrogen or (C₁-C₆)alkyl optionally substituted with from one to three fluorine atoms;

Y, when R¹¹ is present, is selected from O, NR¹³, wherein R¹³ is defined as R¹⁰ is defined above, or (CH₂)_{w} wherein w is an integer from one to six; or

Y, when R¹¹ is absent, is selected from (═O), hydroxy, NR¹³R¹⁴ wherein R¹³ and R¹⁴ are defined as R¹⁰ is defined above, and (CH₂)_{q}CH₃, wherein q is an integer from one to five;

n is an integer from one to three;

z is an integer from one to three; and

R¹¹ is hydrogen, (C₁-C₆)alkyl, —SO₂(C₁-C₆)alkyl, —SO²aryl, aryl, aryl-(C₁-C₆)alkyl, heteroaryl, heteroaryl-(C₁-C₆)alkyl, heterocyclyl, heterocyclyl-(C₁-C₄)alkyl, COR¹⁵, C(O)OR¹⁵, or C(O)NR¹⁵R¹⁶, wherein R¹⁵ and R¹⁶ are independently selected from (C₁-C₆)alkyl, aryl, heteroaryl, heteroaryl-(C₁-C₆)alkyl, aryl-(C₁-C₆)alkyl, heterocyclyl and heterocyclyl-(C₁-C₆)alkyl;

wherein said alkyl moieties within said R¹¹ groups can optionally be substituted with from one to three fluorine atoms, and the aryl, heteroaryl, and heterocyclyl moieties within said R¹¹ groups can optionally be substituted, with one or more substituents, preferably with from zero to two substituents, independently selected from (C₁-C₆)alkyl optionally substituted with from one to three fluorine atoms, (C₁-C₆)alkoxy optionally substituted with from one to three fluorine atoms, cyano, nitro, halo, amino, (C₁-C₆)alkylamino and di-(C₁-C₆)alkylamino; or

R¹¹ is absent;

with the proviso that the sum of n plus z cannot exceed 3;

and the pharmaceutically acceptable salts of such compounds.

Compounds of the formulas 1A and 1B are hereinafter referred to, collectively, as compounds of the formula 1.

More specific embodiments of this invention relate to compounds of the formula 1, and their pharmaceutically acceptable salts, wherein:

J is sulfur and M is nitrogen;

M is nitrogen and J is oxygen;

G is nitrogen;

m is 2;

X is absent;

X is CH(halo);

X is CH(OH);

X is CHNR³R¹²;

X is C(═O);

X is CHOR³;

X is NR³;

X is oxygen;

X is CHNR¹³R¹²;

R¹ and R² are hydrogen;

R¹ and R² are selected from hydrogen and fluoro;

R¹ and R² are selected from hydrogen, methyl, methoxy, chloro and fluoro;

R¹¹ is absent;

R¹¹ is absent and Y is oxo;

YR¹¹ is NR¹³;

YR¹¹ is acetamide;

YR¹¹ is an amide;

Y is NR¹³R¹⁴;

R¹³ is C(═O)(C₁-C₆)alkyl;

R¹⁴ is methyl;

R⁴ is hydrogen and one or both of R² and R³ are hydrogen;

one or both of R² and R³ are hydrogen;

R⁴ and R⁵ are hydrogen;

R¹, R⁵, R⁶, R⁷ and R⁸ are selected, independently, from hydrogen and (C₁-C₃)alkyl;

R⁶, R⁷, R⁸ and R⁹ are hydrogen;

R⁶, R⁷, R⁸ and R⁹ are methyl;

R⁶ and R⁷ and methyl and R⁸ and R⁹ are hydrogen;

R⁶ and R⁷ and hydrogen and R⁸ and R⁹ are methyl; or

R⁶, R⁷, R⁸ and R⁹ are independently selected from hydrogen and methyl.

### DETAILED DESCRIPTION OF THE INVENTION

The term “alkyl”, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “aryl”, unless otherwise indicated, includes an aromatic ring system with no ring heteroatoms (e.g., phenyl or naphthyl).

The term “alkoxy”, unless otherwise indicated, means “alkyl-O—”, wherein “alkyl” is as defined above. Examples of “alkoxy” groups include, but are not limited to, methoxy, ethoxy, propoxy, isopropoxy, butoxy, secbutyloxy and pentoxy.

The term “alkenyl”, unless otherwise indicated, includes unsaturated hydrocarbon radicals having one or more double bonds connecting two carbon atoms, wherein said hydrocarbon radical may have straight, branched or cyclic moieties or combinations thereof. Examples of “alkenyl” groups include, but are not limited to, ethenyl, propenyl, butenyl, pentenyl.

The term “heteroaryl”, unless otherwise indicated, includes monocyclic aromatic heterocycles containing five or six ring members, of which from 1 to 4 are heteroatoms selected, independently, from N, S and O, and bicyclic aromatic heterocycles containing from eight to twelve ring members, of which from 1 to 4 are heteroatoms selected, independently, from N, S and O. Examples of heteroaryl groups include, but are not limited to, furyl, thienyl, triazole, pyridyl, pyrimidinyl, pyrrolyl, imidazolyl, tetrazolyl, oxazolyl and isoxazolyl.

The term “heterocyclyl” or “heterocyclic”, refers to monocyclic saturated or unsaturated nonaromatic ring systems containing 5 or 6 ring members, from 1 to 4 of which are heteroatoms selected, independently, from oxygen, sulfur and nitrogen, and to bicyclic saturated or unsaturated nonaromatic ring systems containing from 10 to 12 ring members, of which from 1 to 4 are heteroatoms selected, independently, from oxygen, sulfur and nitrogen. Examples of heterocyclyl groups include the following: piperidinyl, piperazinyl, morpholinyl, tetrahydrofuryl and tetrahydropyranyl.

The term “one or more substituents”, refers to a number of substituents that equals from one to the maximum number of substituents possible based on the number of available bonding sites.

The terms “halo” and “halogen”, unless otherwise indicated, include, fluoro, chloro, bromo and iodo.

The compounds of formula 1 and the pharmaceutically acceptable salts of these compounds are referred to herein, collectively, as the “compounds of this invention” and the “active compounds of this invention”.

This invention also relates to a pharmaceutical composition comprising a therapeutically effective amount of a compound of the formula 1, or a pharmaceutically acceptable salt thereof, and a pharmaceutically acceptable carrier.

Compounds of the formula 1 may contain chiral centers and therefore may exist in different enantiomeric and diastereomeric forms. This invention relates to all optical isomers and all stereoisomers of compounds of the formula 1, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as optical resolution, fractional crystallization, optically selective reaction, or chromatographic separation in the preparation of the final product or its intermediate. Individual enantiomers of the compounds of formula 1 may have advantages, as compared with the racemic mixtures of these compounds, in the treatment of various disorders or conditions.

In so far as the compounds of formula 1 are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol, ethanol, diethyl ether, dioxane, acetonitrile or tetrahydrofuran. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The present invention also includes isotopically labelled compounds, which are identical to those of formula 1, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, 35S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements and, hence, may be preferred in some circumstances. Isotopically labelled compounds of formula 1 and salts and prodrugs thereof can generally be prepared by carrying out the procedures disclosed in the Schemes and/or in the Examples below, by substituting a readily available isotopically labelled experimental reagent for a non-isotopically labelled reagent.

The compounds of formula 1 have useful pharmaceutical and medicinal properties.

The term “treating”, refers to reversing, alleviating, inhibiting the progress of, or preventing the disorder or condition to which such term applies, or preventing one or more symptoms of such condition or disorder. The term “treatment”, refers to the act of treating, as “treating” is defined immediately above.

This invention also relates to a method of treating a disorder or condition selected from the group consisting of single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound of the formula 1, or a pharmaceutically acceptable salt thereof, that is effective in treating such disorder or condition.

This invention also relates to a pharmaceutical composition for treating a disorder or condition selected from the disorders and conditions as defined in the paragraph directly above, in a mammal in need of such treatment, including a human, comprising an amount of a compound of the formula 1, or a pharmaceutically acceptable salt thereof, that is effective in treating such disorder or condition, and a pharmaceutically acceptable carrier.

A more specific embodiment of this invention relates to the above method and composition wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method and composition wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method and composition wherein the disorder or condition that is being treated is selected from schizophrenia, schizophrenia with concomitant depression or schizophrenia with concomitant anxiety.

Another more specific embodiment of this invention relates to the above method and composition wherein the disorder or condition that is being treated is selected from autism, pervasive development disorder, and attention deficit hyperactivity disorder.

Another more specific embodiment of this invention relates to the above method and composition wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method and composition wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method and composition wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorder, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method and composition wherein the compound of formula 1 is administered to a human for the treatment of any two or more comorbid disorders or conditions selected from those disorders and conditions referred to in any of the above methods.

For the treatment of depression, anxiety, schizophrenia or any of the other disorders and conditions referred to above in the descriptions of the methods and pharmaceutical compositions of this invention, the compounds of this invention can be used in conjunction with one or more other antidepressants or anti-anxiety agents. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine and sertraline. Examples of monoamine oxidase inhibitors include isocarboxazid, pheneizine, and tranylcyclopramine. Suitable reversible inhibitors of monoamine oxidase include moclobemide. Suitable serotonin and noradrenaline reuptake inhibitors of use in the present invention include venlafaxine. Suitable CRF antagonists include those compounds described in International Patent Application Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in World Patent Publication WO 01/77100.

Suitable classes of anti-anxiety agents that can be used in combination with the active compounds of this invention include benzodiazepines and serotonin 1A (5-HT_{1A}) agonists or antagonists, especially 5-HT_{1A} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{1A} receptor agonists or antagonists include buspirone, flesinoxan, gepirone and ipsapirone.

This invention also relates to a method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising administering to said mammal:

(a) a compound of the formula 1, or a pharmaceutically acceptable salt thereof; and

(b) another pharmaceutically active compound that is an antidepressant or anti-anxiety agent, or a pharmaceutically acceptable salt thereof;

wherein the active compounds “a” and “b” are present in amounts that render the combination effective in treating such disorder or condition.

This invention also relates to a pharmaceutical composition for treating a disorder or condition selected from the disorders and conditions as defined in the paragraph directly above, in a mammal in need of such treatment, including a human, comprising:

(a) a compound of the formula 1, or a pharmaceutically acceptable salt thereof;

(b) another pharmaceutically active compound that is an antidepressant or anti-anxiety agent, or a pharmaceutically acceptable salt thereof; and

(c) a pharmaceutically acceptable carrier;

wherein the active compounds “a” and “b” are present in amounts that render the composition effective in treating such disorder or condition.

A more specific embodiment of this invention relates to the above method and composition wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method and composition wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method and composition wherein the disorder or condition that is being treated is selected from schizophrenia, schizophrenia with concomitant depression or schizophrenia with concomitant anxiety.

Another more specific embodiment of this invention relates to the above method and composition wherein the disorder or condition that is being treated is selected from autism, pervasive development disorder, and attention deficit hyperactivity disorder.

Another more specific embodiment of this invention relates to the above method and composition wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method and composition wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method and composition wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorder, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method and composition wherein the compound of formula 1 and the additional antidepressant or anti-anxiety agent are administered to a human for the treatment of any two or more comorbid disorders or conditions selected from those disorders and conditions referred to in any of the above methods.

The active compounds of this invention may be prepared as described in the following reaction schemes. Unless otherwise indicated, Y, J, M, G, n, z, m, q, X, and R¹ through R¹⁶ in the reaction schemes and discussion that follow, are as defined above.

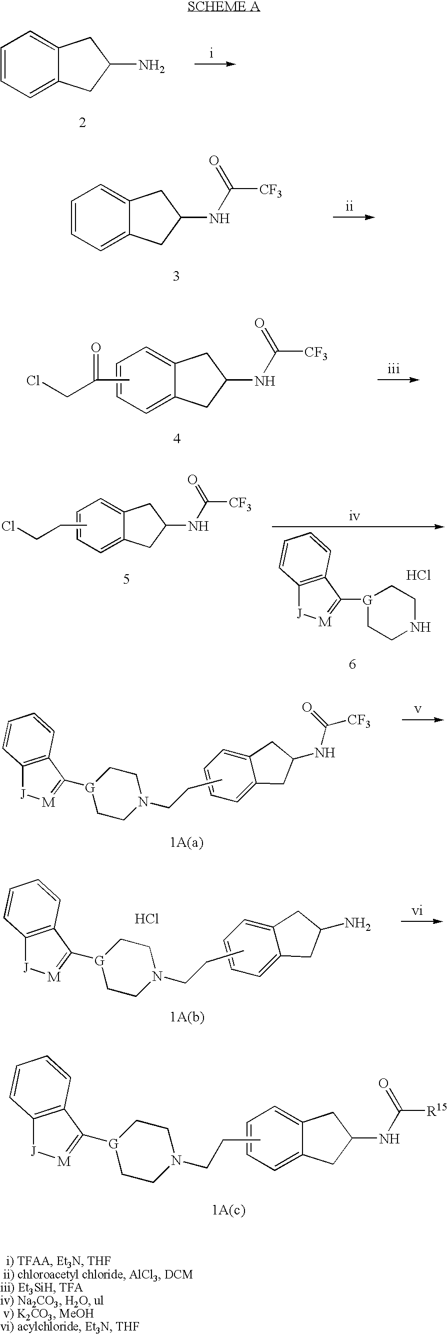

Scheme A illustrates a method of synthesizing compounds of the formula 1A wherein YR¹¹ is amino (compounds of the formula 1A(b)), trifluoroacetamide (compounds of the formula 1A(a)), and NHC(O)R¹⁵ (compounds of the formula 1A(c)). Steps i, ii and iii of Scheme A are carried out as described in Preparations 1, 2, and 3 in the experimental examples section of this application. Compounds of the formula 1A(a) can be prepared by reacting a compound of the formula 5 with a compound of the formula 6 in the presence of a base such as Na₂CO₃, potassium carbonate, cesium carbonate, sodium bicarbonate, potassium bicarbonate, triethylamine, pyridine or any other organic or inorganic base that is suitable for quenching acid, preferably sodium carbonate, in a polar solvent such as water, acetonitrile, tetrahydrofuran (THF), dimethylformamide (DMF), dioxane, dimethylsulfoxide (DMSO), or a mixture two or more of the foregoing solvents, preferably in water, at a temperature from about 40° C. to about 200° C., preferably at about 175° C. under microwave assistance for about 10 to 180 minutes. This reaction can also be carried out at about 100° C. using conventional heating for about 1-96 hours, preferably for about 24-48 hours.

Compounds of the formula 1A(a) can be converted into the corresponding amines of formula 1A(b) by reacting them with potassium carbonate, sodium carbonate, sodium hydroxide, potassium hydroxide, lithium hydroxide, barium hydroxide or triethylamine, preferably potassium carbonate, in a polar solvent such as water, ethanol, propanol or methanol, or a mixture of water and methanol, at a temperature from about 25° C. to about the reflux temperature of the solvent, preferably at about the reflux temperature. This reaction is preferably conducted in a mixture of water and methanol at about 60° C.

Reaction of a compound of the formula 1A(b) with the appropriate acylchloride or sulfonyl chloride, or with isocyanate, in the presence of an inorganic base such as potassium carbonate or sodium carbonate, or an organic base such as triethylamine (TEA), pyridine, or diisopropylethylamine, in an ethereal solvent such as THF, dioxane, diglyme, or ethyl ether, or a chlorinated hydrocarbon solvent such as chloroform (CHCl₃), dichloroethane or methylene chloride (CH₂Cl₂), at a temperature from about 0° C. to about the reflux temperature of the reaction mixture, preferably at about room temperature, yields the corresponding compound of formula 1A(c). Preferably, this reaction is carried out in THF in the presence of triethylamine at about room temperature.

Compounds of the formula 1A(a), 1A(b), and 1A(c) wherein 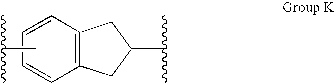
is replaced by 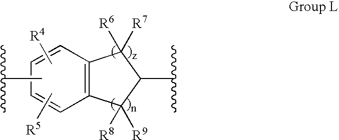
can be prepared using methods analogous to those depicted in Scheme A, as known to those of skill in the art, from the appropriate starting materials of the formula 2′.

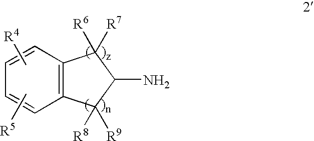

Compounds of the formula 1B wherein YR¹¹ is amino, trifluoroacetamide and NHC(O)R¹⁵ can be prepared using methods analogous to those depicted in Scheme A, that will be within the skill of one in the art, from the appropriate starting materials of the formula 2″.

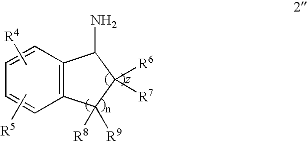

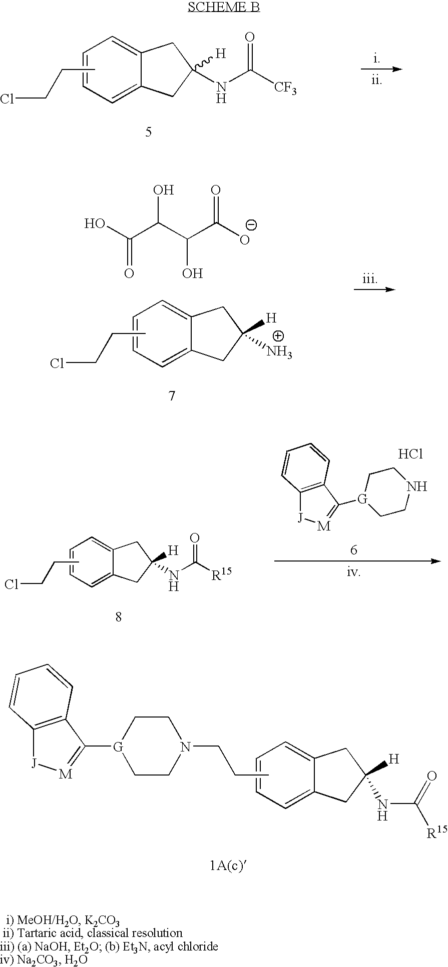

Scheme B illustrates a method of preparing compounds of the formula 1A(c) having the stereochemistry depicted in Scheme B at the carbon to which YR¹¹ is attached. Such compounds are hereinafter referred to as compounds of the formula 1A(c)′.

Steps i, ii, and iii of Scheme B are described in Preparations 7, 8, and 9 in the experimental examples section of this application. The preparation of compounds of the formula 1A(c)′ from compounds of the formula 8 can be accomplished using the procedure described above for preparing compounds of the formula 1A(a) from compounds of the formula 5 in Scheme A.

Compounds of the formula 1A(c)′ wherein group K, as defined above, is replaced by group L, as defined above, can be prepared using methods analogous to those depicted in Scheme B, from the appropriate starting materials, that will be know to one of skill in the art.

Compounds of the formula 1B wherein YR¹¹ is defined as for compounds of the formula 1A(c)′ in Scheme B can be prepared using methods analogous to those depicted in Scheme B, from starting materials identical to those of the formula 5, as depicted in Scheme B, except that the trifluoroacetamide substituent in such compounds is attached to a carbon atom adjacent to the benzo ring.

Compounds of the formulas 1A(a) and 1A(b) wherein Y is NR¹³ and R¹³ is hydrogen can be converted into the corresponding compounds wherein Y is NR¹³ and R¹³ is other than hydrogen, by reacting such compounds with the appropriate compound of the formula X¹R¹³ wherein X¹ is a leaving group such as halo, mesylate, or tosylate, preferably iodo, in the presence of a base such as sodium hydride (NaH), potassium hydride (KH), sodium methoxide (NaOCH₃), or potassium t-butoxide (KOt-Bu). This reaction is typically conducted in an ethereal solvent such as THF, ethyl ether, dioxane, or diglyme, at a temperature from about room temperature to about the reflux temperature of the reaction mixture. It is preferably carried out in THF, using KOt-Bu as the base, at the reflux temperature.

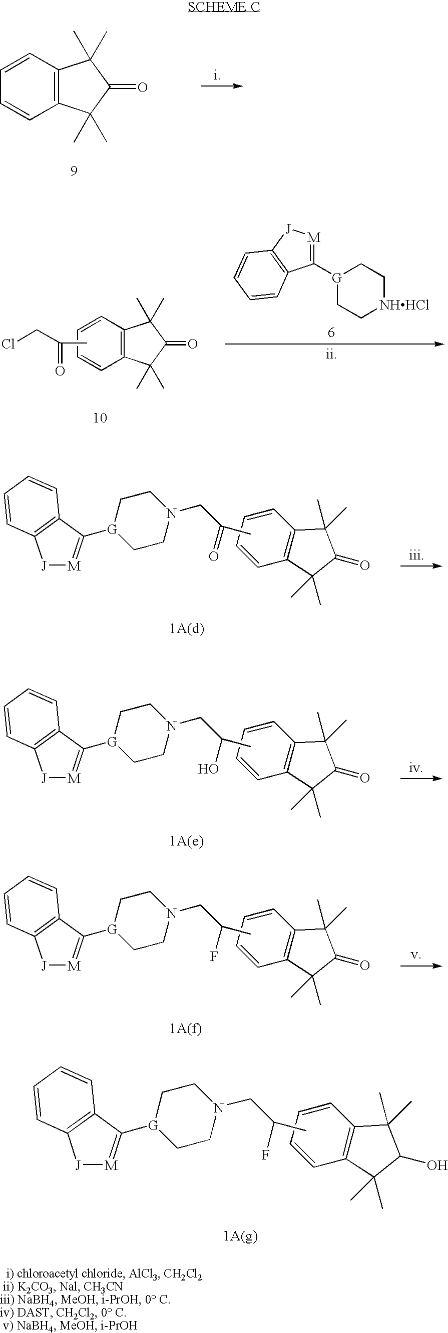

Scheme C illustrates a method that can be used to prepare compounds of the formulas 1A and 1B wherein R¹¹ is absent, Y is oxo (═O) or hydroxy (OH), and X is C(═O), CHOH, or CH(halo). Although Scheme D depicts this process only for a subgenus of compounds of the formula 1A, analogous processes that will be within the skill of one skilled in the art can be used to prepare all compounds of formulas 1A and 1B wherein R¹¹ is absent, Y is (═O) or OH, and X is C(═O), CHOH, or CH(halo).

Step i of Scheme C is carried out as described in Preparation 22 in the experimental examples section of this application. Compounds of the formula 10 are reacted with the appropriate compounds of the formula 6 to form the corresponding compounds of the formula 1A(d). This reaction is typically carried out in a solvent such as acetonitrile, THF, dioxane, DMF, DMSO, dichloromethane, diethyl ether, methanol or ethanol, preferably in acetonitrile, in the presence of potassium carbonate, sodium carbonate, cesium carbonate, triethylamine, diethylisopropylamine, pyridine or tert-butoxide, and sodium iodide or potassium iodide, at a temperature from about 0° C. to about the reflux temperature of the reaction mixture. Preferably, the reaction is carried out in the presence of potassium carbonate and sodium iodide at about room temperature.

Reduction of the compounds of formula 1A(d) yields the corresponding compounds of formula 1A(e). This reduction can be accomplished using sodium borohydride (NaBH₄) or lithium borohydride (LiBH₄), preferably NaBH₄, in a hydroxylated solvent such as a (C₁-C₆)alkanol, or a mixture of such solvents, at a temperature between 0° C. and room temperature. A mixture of methanol and isopropanol is the preferred solvent. The preferred temperature is about 0° C.

The corresponding compounds of formula 1A(f) can be prepared by reacting the compounds of formula 1A(e) formed in the above step with (diethylamino)sulfur trifluoride (DAST) in a chlorinated hydrocarbon solvent such as chloroform, dichloroethane or methylene chloride, preferably in methylene chloride, at a temperature from about 0° C. to about room temperature, preferably at about 0° C. Reduction of the resulting compounds of the formula 1A(f), using the methods set forth above for step iii in Scheme C, yields the corresponding compounds of formula 1A(g).

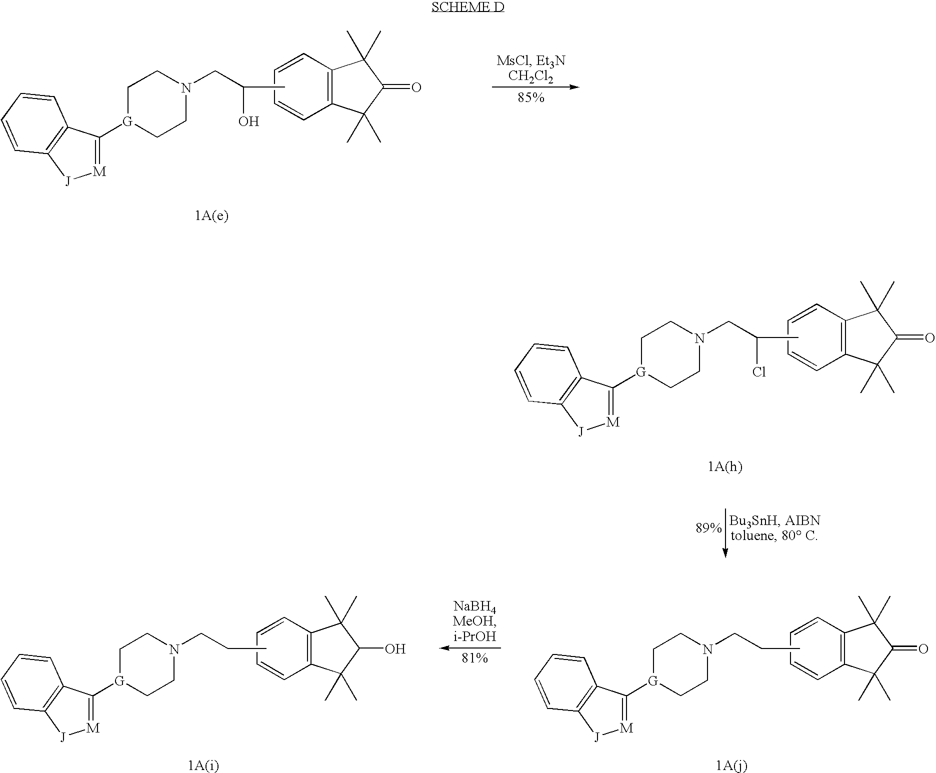

Scheme D illustrates the synthesis of compounds of the formula 1 wherein X is CH(halo) from the corresponding compounds wherein X is CH(OH), and of synthesizing compounds of the formula 1A wherein X is absent from the corresponding compounds wherein X is CH(halo). While these methods are illustrated for certain subgenera of compounds of the formula 1A, they are applicable to all compounds of the formula 1.

Referring to Scheme D, compounds of the formula 1A(e) are reacted with Ts(halo) or Ms(halo), wherein Ms is mesyl and Ts is tosyl, in a chlorinated hydrocarbon solvent such as chloroform (CHCl₃), dichloroethane (DCE), or methylene chloride (CH₂Cl₂), or in an ethereal solvent such as diethyl ether, dioxane or THF, at a temperature from about 0° C. to about room temperature, preferably at about room temperature, in the presence of a base, to yield the corresponding compounds of the formula 1A(h). Suitable bases include tertiary organic bases such as triethylamine (TEA, Et₃N), pyridine, or diethylaminopyridine. Triethylamine is the preferred base. Methylene chloride is the preferred solvent.

The resulting compounds of formula 1A(h) can be converted into the corresponding compounds of the formula 1A(j) by reacting them with tributyl tin hydride (Bu₃SnH) in the presence of a catalyst that is a radical initiator such as benzoyl peroxide or azobisisobutyronitrile (AIBN), preferably AIBN, in an aromatic hydrocarbon solvent such as benzene, toluene or xylene, preferably toluene. Suitable reaction temperatures range from about room temperature to about the reflux temperature of the reaction mixture. The reflux temperature is preferred. Reduction of compounds of the formula 1A(j), using the procedure described above for reducing compounds of the formula 1A(d) in Scheme C, yields the corresponding compounds of formula 1A(i).

Compounds of the formula 1 wherein X is CH(halo) can be converted into the corresponding compounds of the formula 1 wherein X is CHNR³R¹² by reacting them with a compound of the formula NHR³R¹² in an ethereal solvent such as THF, diglyme, dioxane, DMF, DMSO, acetonitrile or ethyl ether, at a temperature from about room temperature to about the reflux temperature of the reaction mixture, preferably at about the reflux temperature.

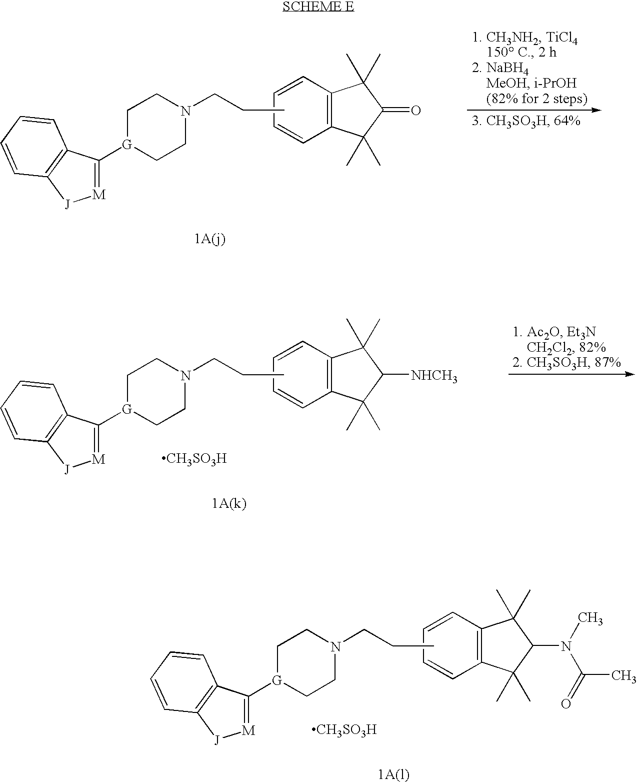

Scheme E illustrates the synthesis of compounds of the formula 1 wherein YR¹¹ is NHCH₃ or N(CH₃)C(═O)CH₃. Referring to Scheme E, compounds of the formula 1A(k) can be prepared as follows. A compound of the formula 1A(j) is reacted with methylamine in the presence of a Lewis acid such as aluminum trichloride or titanium tetrachloride, preferably titanium tetrachloride. This reaction is generally conducted in an aromatic hydrocarbon solvent such as toluene, zylene or benzene, preferably toluene, at a temperature from about 80° C. to about 150° C., preferably at about 150° C. The product of this reaction is then reacted with a reducing agent such as NaBH₄, LiBH₄, sodium cyanoborohydride (NaCNBH₃) or KBH₄, preferably NaBH₄, in a hydroxylated solvent such as a (C₁-C₆)alkanol, or a mixture of two or more such solvents, at a temperature between 0° C. and room temperature. A mixture of methanol and isopropanol is the preferred solvent. The preferred temperature is about 0° C. The resulting free base of the compound of formula 1A(k) can be converted into the corresponding methanesulfonate salt by reacting it with methanesulfonic acid using methods well known to those of skill in the art.

Reaction of the compound of formula 1A(k) with acetic anhydride or acetyl chloride yields the desired compound of formula 1A(l). The reaction with acetic anhydride is typically carried out in a chlorinated hydrocarbon solvent such as methylene chloride, chloroform or dichloroethane, or in an ethereal solvent such as THF, diglyme or ethyl ether, at a temperature from about 0° C. to about the reflux temperature of the reaction mixture. Preferably, the reaction is carried out in a methylene chloride solvent at about room temperature.

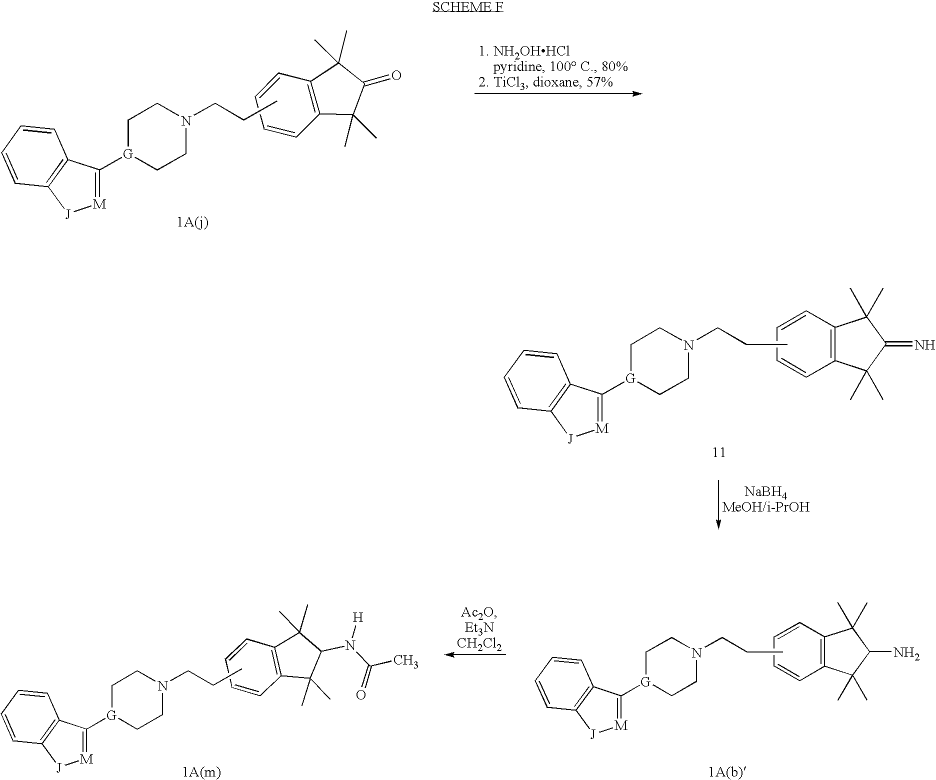

Scheme F depicts a method of synthesizing compounds of the formula 1 wherein YR¹¹ is NHC(═O)CH₃. While depicted only for a subgenus of compounds of the formula 1A, analogous methods that will be within the skill of one skilled in the art can be used to prepare all compounds of the formula 1 wherein YR¹¹ is NHC(═O)CH₃.

Referring to Scheme F, a compound of the formula 1A(j) is reacted with hydroxylamine hydrochloride (NH₂OH.HCl) in a tertiary amine base solvent such as pyridine or triethylamine. Pyridine is preferred. Suitable reaction temperatures range from about room temperature to about the reflux temperature of the reaction mixture, with the reflux temperature being preferred. Treatment of the product from this reaction with titanium trichloride in an ethereal solvent such as dioxane, ethyl ether, diglyme or THF, preferably dioxane, at a temperature from about 0° C. to about the reflux temperature of the reaction mixture, preferably at about room temperature, yields the corresponding compound of formula 11. Reduction of the compound of formula 11 using the procedure described above for reducing compounds of the formula 1A(d) in Scheme C, yields the corresponding compounds of the formula 1A(b)′.

Compounds of the formula 1A(b)′ can be converted into the corresponding compounds of formula 1A(m) using the procedure described above in Scheme E for preparing the free base of compounds of the formula 1A(l).

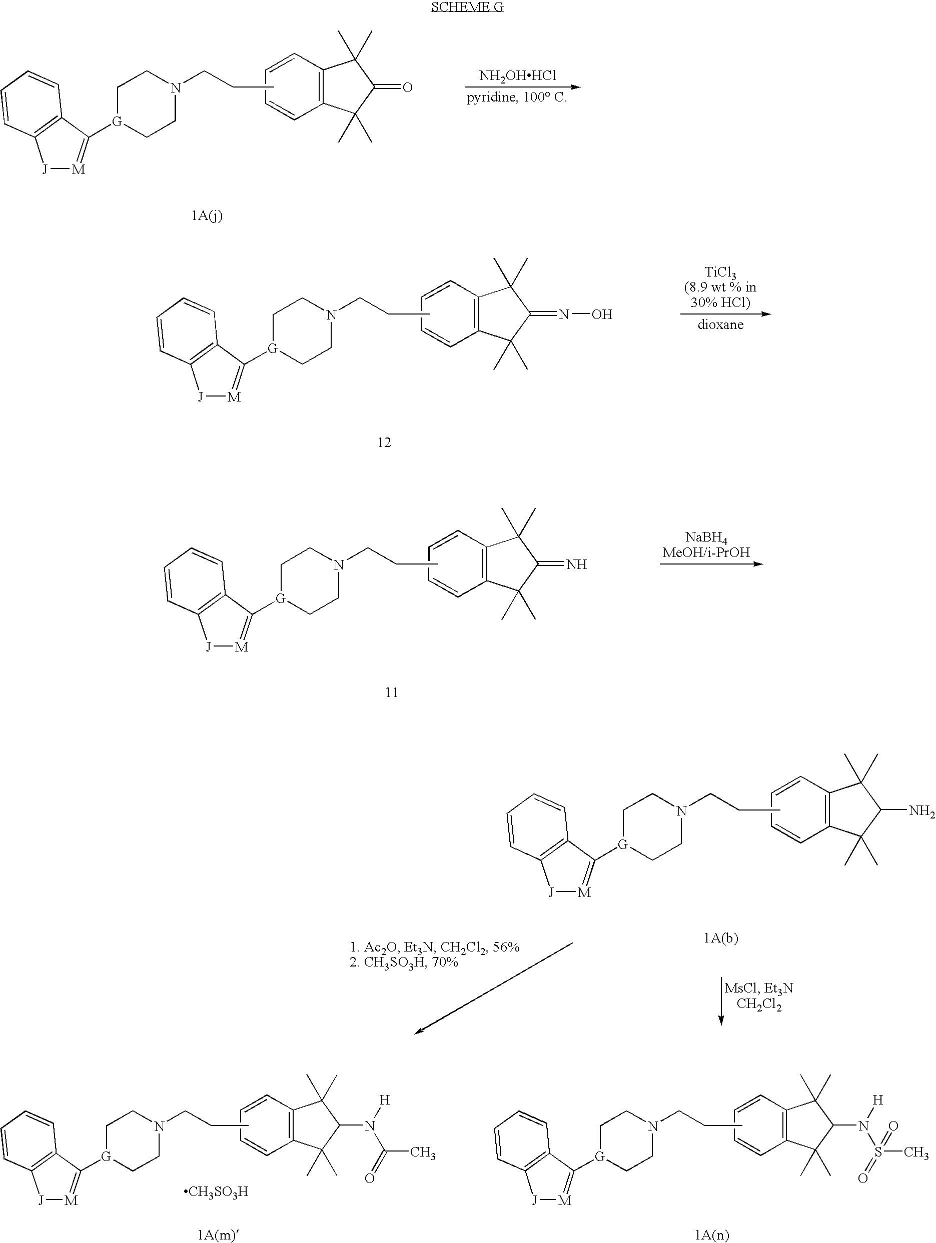

The synthesis of compounds of the formula 1 wherein YR¹¹ is NHSO₂CH₃ is depicted in Scheme G. While only a subgenus of compounds of the formula 1A is shown in the scheme, the synthetic procedures of Scheme G can be used to prepare all compounds having formula 1 wherein YR¹¹ is NHSO₂CH₃.

Referring to Scheme G, compounds of the formulas 12 and 11 can be prepared as described in Examples 110 and 111, respectively, in the experimental examples section of this application. Reduction of the compounds of formula 11 using the procedure described above for reducing compounds of the formula 1A(d) in Scheme C yields the corresponding compounds of formula 1A(b). The resulting compounds of formula 1A(b) can be converted into the corresponding compounds having formula 1A(n) using the procedure described above for preparing compounds of the formula 1A(m) in Scheme F, with the exception that the acyl derivative reactant is mesyl chloride instead of acetic anhydride or acetyl chloride.

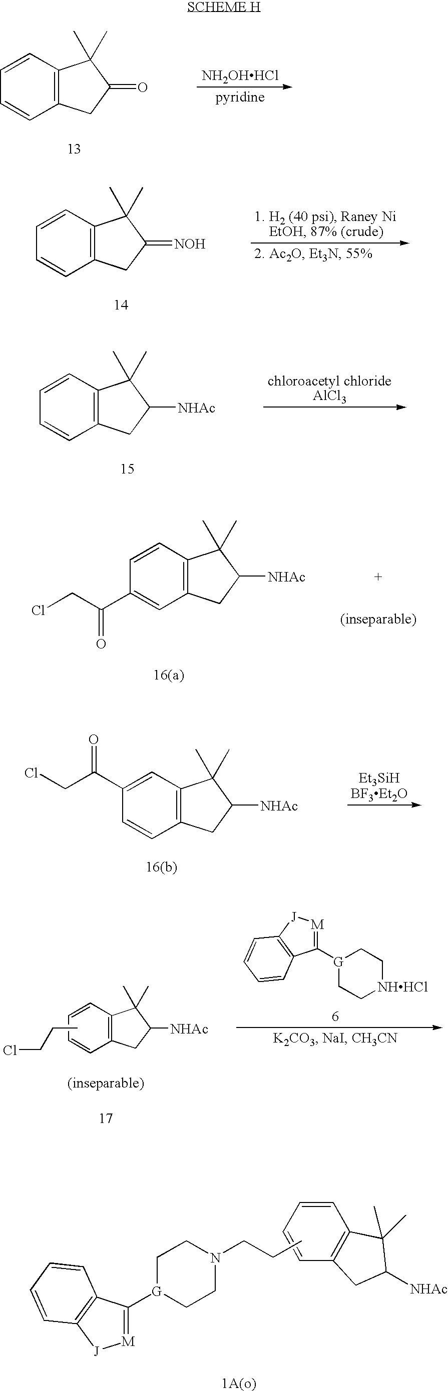

Scheme H depicts an alternative method of synthesizing compounds of the formula 1 wherein YR¹¹ is NHC(═O)R¹⁵. Referring to Scheme H, procedures for making chemical intermediates 14, 15, 16(a), 16(b) and 17 are described in Preparations 30 through 33 of the experimental examples section of this application. Compounds of the formula 1A(o) can be prepared using methods analogous to those described above for the preparation of compounds of the formula 1A(d) in Scheme C.

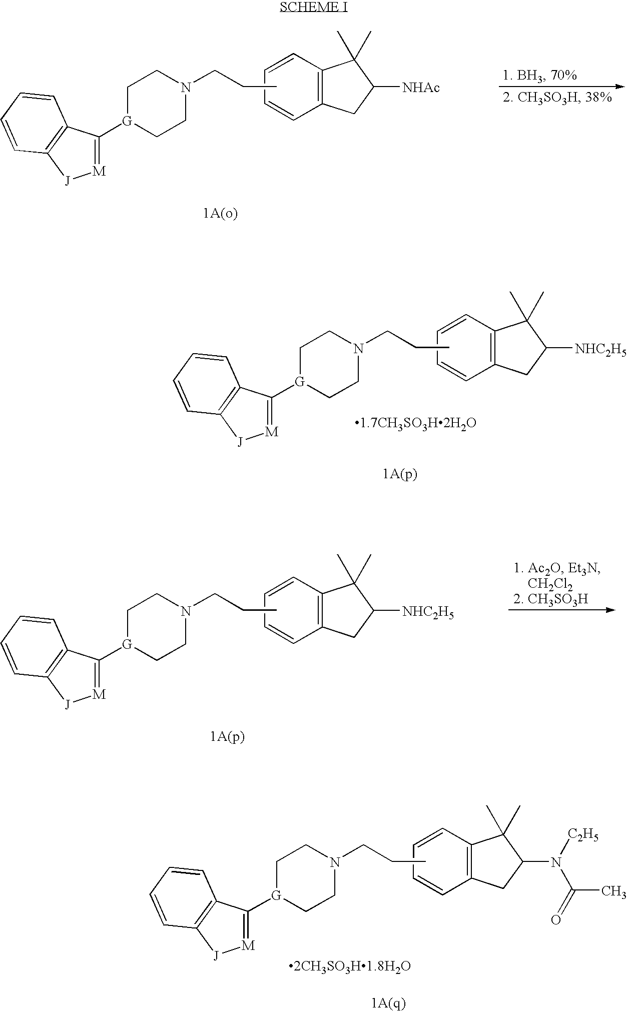

Compounds of the formulas 1A(p) and 1A(q) can be prepared as illustrated in Scheme I. Referring to Scheme I, a compound of the formula 1A(o) is reduced using a boron hydride such as diborane or lithium aluminum hydride, preferably diborane, to yield the corresponding compounds of formula 1A(p). Typically, this reaction is carried out in an ethereal solvent such as THF, diglyme, dioxane or ethyl ether, preferably in THF, at a temperature from about room temperature to about the reflux temperature of the reaction mixture, preferably at about the reflux temperature. Formation of the methanesulfonate hydrate of formula 1.7CH₃SO₃H.2H₂O is described in Example 117 in the experimental examples section of this application. Reaction of the resulting compound of formula 1A(p) with acetic anhydride or acetyl chloride, in a chlorinated hydrocarbon solvent such as CHCl₃, CH₂Cl₂ or trichloroethane, preferably CH₂Cl₂, at a temperature from about 0° C. to about the reflux temperature of the reaction mixture, preferably at about room temperature yields the corresponding compound of formula 1A(q). Formation of the methanesulfonate hydrate of the formula 2CH₃SO₃H.1.8H₂O is described in Example 118 in the experimental examples section of this application.

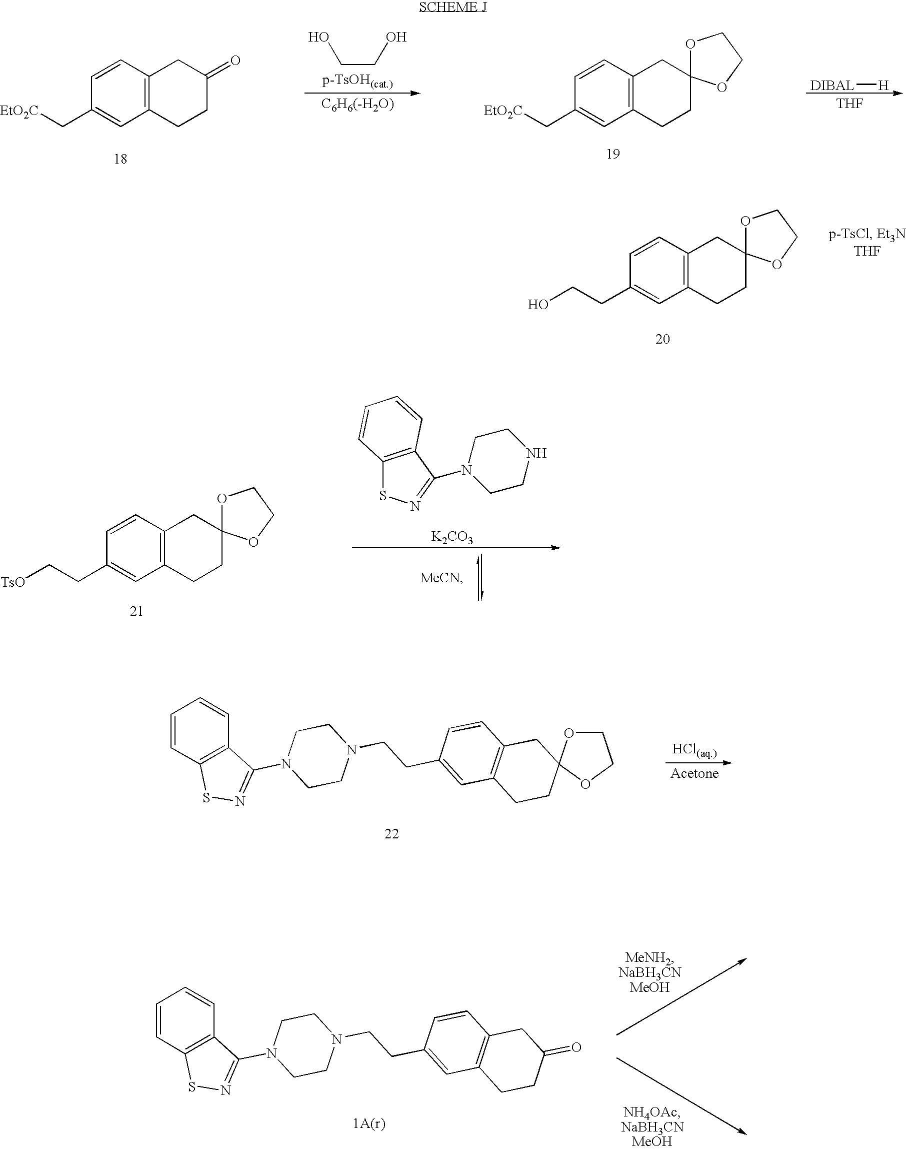

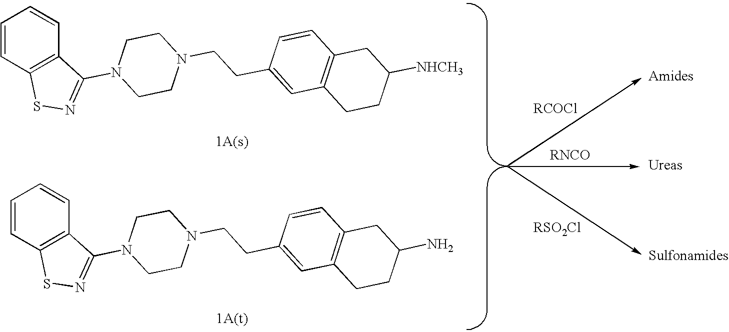

Scheme J illustrates the synthesis of exocyclic amido tetralin derivatives of the formulas 1A(r), 1A(s) and 1A(t). Analogous compounds of the formulas 1A and 1B wherein J, M, G, X, Y, m and R¹ through R¹¹ have any of the meanings set forth above in the definition of compounds of the formulas 1A and 1B, and wherein one of n and z is one and the other is two, can be prepared using methods that will be within the skill of one skilled in the art in view of the synthetic methods described above and in experimental Examples 1 through 172. Examples of the synthesis of such tetralin derivatives are set forth in experimental Examples 121 through 172.

Referring to Scheme J, the preparation of compound 19 is described in Preparation 34 in the experimental Examples section of this application. The preparation of compounds 20, 21 and 22 is described, respectively, in Preparation 35, Preparation 36 and Example 121 in the experimental Examples section of this application. The synthesis of compound 1A(r) is described in Example 122. Compound 1A(r) can also be prepared using methods similar to those described above in Schemes C and D, starting with the appropriate analogous starting materials. The syntheses of compounds 1A(t) and 1A(s) are exemplified, respectively, in Examples 123 and 124. These compounds can also be prepared using methods similar to those described in Scheme F, starting with the appropriate analogous starting materials.

The preparation of other compounds of the formula 1 not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience. Under microwave assisted heating, sealed reactors are indicated, resulting in high pressure reactions up to as much as 350 psi.

The compounds of the formula 1, and the intermediates shown in the above reaction schemes can be isolated and purified by conventional procedures, such as recrystallization or chromatographic separation.

The compounds of the formula 1, and their pharmaceutically acceptable salts, can be administered to mammals via either the oral, parenteral (such as subcutaneous, intravenous, intramuscular, intrasternal and infusion techniques), rectal, buccal or intranasal routes. In general, these compounds are most desirably administered in doses ranging from about 3 mg to about 600 mg per day, in single or divided doses (i.e., from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 25 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

The compounds of the present invention may be administered alone or in combination with pharmaceutically acceptable carriers or diluents by any of the routes previously indicated, and such administration may be carried out in single or multiple doses. More particularly, the therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

This invention relates to methods of treating anxiety, depression, schizophrenia and the other disorders referred to in the description of the methods of the present invention, wherein a compound of this invention and one or more of the other active agents referred to above (e.g., an NK1 receptor antagonist, tricyclic antidepressant, 5HT1D receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the compounds of this invention, when used as a single active agent or in combination with another active agent, will be administered to an adult human in an amount from about 3 mg to about 300 mg per day, in single or divided doses, preferably from about 25 to about 100 mg per day. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.1 mg to about 2000 mg, preferably from about 1 mg to about 200 mg of the 5HT reuptake inhibitor per unit dose, which could be administered, for example, 1 to 4 times per day. A proposed daily dose of a 5HT1D receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1D receptor antagonist per unit dose, which could be administered, for example, 1 to 4 times per day.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

All of the title compounds of the examples were tested and at least one stereoisomer of each such compound exhibited a binding affinity for the D2 receptor, measured as percent inhibition at a concentration of 0.1 μm, of no less than 14% and up to 100%. At least one stereoisomer of each such compound exhibited a binding affinity for the 5HT2 receptor, measured as percent inhibition at a concentration of 0.1 μm, of no less than 80% and up to 100%.

The ability of the compounds of this invention to bind to the dopamine D2 and serotonin 2A (5HT2A) receptors can be determined using conventional radioligand receptor binding assays. All receptors can be heterologously expressed in cell lines and experiments conducted in membrane preparations from the cell lines using procedures outlined below. IC₅₀ concentrations can be determined by nonlinear regression of concentration-dependent reduction in specific binding. The Cheng-Prussoff equation can be used to convert the IC₅₀ to Ki concentrations.

Dopamine D2 Receptor Binding:

[³H]Spiperone binding to a membrane preparation from CHO-hD2L cells is carried out in 250 μl of 50 mM Tris-HCl buffer containing 100 mM NaCl, 1 mM MgCl₂ and 1% DMSO at pH 7.4. Duplicate samples containing (in order of addition) the test compounds, 0.4 nM [³H]spiperone and approximately 12 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-120 were tested using the above assay, in which specific binding determined in the presence of 1 mM haloperidol was 95%. All of the title compounds of Examples 1-120 exhibited Ki values less than or equal to 1 uM. The title compound of Example 58 exhibited a Ki of 3 nM. The title compound of Example 56 exhibited a Ki of 5 nM. The title compound of Example 60 exhibited a Ki of 9nM.

Serotonin 2A Binding:

[³H]Ketanserin binding to Swiss-h5HT2A cell membranes can be carried out in 250 μl of 50 mM Tris-HCl buffer pH 7.4. Duplicate samples containing (in order of addition) test compounds, 1.0 nM [³H]ketanserin, and approximately 75 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-120 were tested using the above assay, in which specific binding determined in the presence of 1 mM ketanserin was 90%. All of the title compounds of Examples 1-120 exhibited Ki values less than or equal to 1 uM. The title compound of Example 58 exhibited a Ki of 0.03 nM. The title compound of Example 56 exhibited a Ki of 0.55 nM. The title compound of Example 60 exhibited a Ki of 0.09 nM.

The following Examples illustrate the preparation of the compounds of the present invention. Melting points are uncorrected. NMR data are reported in parts per million and are referenced to the deuterium lock signal from the sample solvent.

### EXAMPLES

### Preparation 1

### 2,2,2-TRIFLUORO-N-INDAN-2-YL-ACETAMIDE

To a solution of 2-aminoindane (7.89 g, 69.71 mmole, 1 eq) in anhyd. THF (100 ml) and triethylamine (19.43 ml, 139.42 mmole, 2.0 eq) at <5° C. (ice/acetone), was added dropwise a solution of trifluoroaceticanhydride (TFAA) (14.77 ml, 104.56 mmole, 1.5 eq), in anhyd. THF (20 ml) keeping the reaction temperature <10° C. After addition of TFAA, the reaction was allowed to warm to room temperature (rt) and stirred for 30 min. The reaction was diluted with H₂O (100 ml)/ethyl acetate (100 ml) and the layers separated. The organics were washed with 2N HCl (2×100 ml), brine (100 ml), dried (MgSO₄) and concentrated to a dark liquid residue. The residue was taken up in MeOH (75 ml) and desired product was precipitated with water to give green/brown crystals. Upon drying in vacuo at 60° C. for 1 h, titled product was obtained (13.56 g, 59.16 mmole, 85% yield). ¹H NMR (400 MHz, CDCl₃) δ7.30-7.15 (m, 4H), 6.44 (bs, 1H), 4.81-4.71 (M, 1H), 3.40 (d, J=7.1 Hz, 1H), 3.35 (d, J=7.1 Hz, 1H), 2.90 (d, J=4.1 Hz, 1H), 2.86 (d, J=4.1 Hz, 1H).

### Preparation 2

### N-[5-(2-CHLORO-ACETYL)-INDAN-2-YL]-2,2,2-TRIFLUORO-ACETAMIDE

To a solution of 2,2,2-Trifluoro-N-indan-2-yl-acetamide (11.75 g, 51.26 mmole, 1.0 eq) in carbondisulfide (125 ml) was added aluminum trichloride (27.34 g, 205.04 mmole, 4.0 eq) at room temperature. To the stirring slurry was added portionwise chloroacetylchloride (6.12 ml, 76.90 mmole, 1.5 eq) with noted HCl evolution. After stirring for 15 min at rt, the reaction was heated to reflux for 1.5 h followed by cooling to rt. The solvent was decanted and the residue was carefully quenched with cold water. The precipitate was filtered off and recrystallized from 2-propanol/water and dried in vacuo @ 60° C. o/n to give titled product as an olive solid (13.43 g, 43.93 mmole, 86% yield). ¹H NMR (400 MHz, CDCl₃) δ7.84 (s, 1H), 7.81 (d, J=8.0 Hz, 1H), 7.36 (d, J=8.0 Hz, 1H), 6.43 (bs, 1H), 4.86-4.76 (m, 1H), 4.66 (s, 2H), 3.46 (d, J=7.2 Hz, 1H), 3.42 (d, J=7.2 Hz, 1H), 2.98 (d, J=4.5 Hz, 1H), 2.94 (d, J=4.5 Hz, 1H).

### Preparation 3

### N-[5-(2-CHLORO-ETHYL)-INDAN-2-YL]-2,2,2-TRIFLUORO-ACETAMIDE

To a solution of N-[5-(2-Chloro-acetyl)-indan-2-yl]-2,2,2-trifluoro-acetamide (13.43 g, 43.93 mmole, 1.0 eq) in TFA (100 ml) was added triethylsilylhydride (17.5 ml, 109.82 mmole, 2.5 eq) at rt. The reaction was heated to 60° C. for 20 min. The reaction was cooled and poured into cold water. The resulting solid was collected by filtration and crystallized from MeOH/H₂O to give desired product in 87% yield. ¹H NMR (400 MHz, CDCl₃) δ7.19 (d, J=7.6 Hz, 1H), 7.11 (s, 1H), 7.06 (d, J=7.6 Hz, 1H), 6.42 (bs, 1H), 4.83-3.73 (m, 1H), 3.70 (t, J=6.95, 2H), 3.40-3.30 (m, 2H), 3.04 (t, J=6.95 Hz, 1H), 2.87 (dd, J₁=4.5 Hz, J₂=4.9 Hz, 1H), 2.83 (dd, J₁=4.4 Hz, J₂=4.9 Hz, 1H).

### Example 1

### N-{5-[2-(4BENZO[D]ISOTHIAZOL-3-YL-PIPERAZIN-1-YL)-ETHYL]-INDAN-2-YL}-2,2,2-TRIFLUORO-ACETAMIDE

N-[5-(2-Chloro-ethyl)-indan-2-yl]-2,2,2-trifluoro-acetamide (3.00 g, 10.28 mmol), 3-piperazin-1-yl-benzo[d]isothiazole hydrochloride (5.26 g, 20.57 mmol) and sodium carbonate (2.18 g, 20.57 mmol) in H₂O (20 mL) was subjected to 175° C. for 10 min. under microwave assistance using a CEM MARS-5 microwave. The reaction was diluted with H₂O (50 mL) and EtOAc (100 mL). The layers were separated and the organics washed with 4N HCl (2×25 mL). The aqueous layer was made basic and extracted with CH₂Cl₂ (3×50 mL). The organics were dried (MgSO₄), and concentrated to a solid residue. The residue was subjected to chromatography (3% MeOH/CH₂Cl₂). N-{5-[2-(4-Benzo[d]isothiazol-3-yl-piperazin-1-yl)-ethyl]-indan-2-yl}-2,2,2-trifluoro-acetamide (3.20 g), was isolated in 100% purity @ 254 nm; LCMS (APCI): 475 [M+H]⁺. ¹H NMR (400 MHz, CDCl₃) δ 7.90 (d, J=8.2 Hz, 1H), 7.80 (d, J=8.2 Hz, 1H), 7.46 (t, J=7.4 Hz, 1H), 7.34 (t, J=7.4 Hz, 1H), 7.17 (d, J=7.4 Hz, 1H), 7.12 (s, 1H), 7.08 (d, J=7.4 Hz, 1H), 6.46 (bs, 1H), 4.82-4.71 (m, 1H), 3.63-3.55 (m, 4H), 3.40-3.29 (m, 2H), 2.90-2.80 (m, 4H), 2.79-2.72 (m, 4H), 2.71-2.65 (m, 2H).

### Example 2

### 5-[2-(4-BENZO[D]ISOTHIAZOL-3-YL-PIPERAZIN-1-YL)-ETHYL]-INDAN-2-YLAMINE HYDROCHLORIDE SALT

To a solution of N-{5-[2-(4-Benzo[d]isothiazol-3-yl-piperazin-1-yl)-ethyl]-indan-2-yl}-2,2,2-trifluoro-acetamide (2.89 g, 6.09 mmole), in MeOH/H₂O (100 ml, 1:1), was added K₂CO₃ (2.17 g, 30 mmole) and the whole heated to 60° C. for 2H. The organics were evaporated and the aqueous extracted with dichloromethane (DCM) (4×50 ml). The organics were dried (MgSO₄), concentrated and the residue purified by chromatography (10% MeOH/DCM with 1% NH₄OH) to give desired product (1.45 g, 3.83 mmole). The product was taken up in and treated with 1N HCl in Et₂O. The HCl salt was isolated by filtration and dried in vacuo at 50° C. in 100% purity @ 254 nm; LCMS (APCI): 379 [M+H]⁺. 1H NMR (400 MHz, DMSO-D6) δ ppm 2.99 (d, J=8.40 Hz, 1 H) 3.09 (d, J=16.80 Hz, 2 H) 3.20 (m, 3 H) 3.32 (d, J=11.92 Hz, 4 H) 3.51 (s, 4 H) 3.57 (s, 1 H) 3.62 (s, 2 H) 3.94 (dq, J=12.26, 6.14 Hz, 1 H) 4.05 (d, J=13.29 Hz, 2 H) 5.07 (s, 1 H) 7.09 (d, J=7.62 Hz, 1 H) 7.20 (m, 2 H) 7.44 (t, J=7.42 Hz, 1 H) 7.57 (t, J=7.52 Hz, 1 H) 8.10 (dd, J=13.19, 8.30 Hz, 2 H) 8.41 (s, 3 H) 11.71 (s, 1 H).

### Example 3

### N-{5-[2-(4-BENZO[D]ISOTHIAZOL-3-YL-PIPERAZIN-1-YL)-ETHYL]-INDAN-2-YL}-ACETAMIDE

To a solution of 5-[2-(4-Benzo[d]isothiazol-3-yl-piperazin-1-yl)-ethyl]-indan-2-ylamine (1.00 g, 2.41 mmole) and Et₃N (1.08 ml, 7.23 mmole) in dry THF (10 ml) was added acetyl chloride (0.26 ml, 3.61 mmole). The reaction was stirred at rt for 1 h. then quenched with water (25 ml). The reaction was diluted with EtOAc (50 ml) and the layers separated. The organics were dried (MgSO₄), concentrated and the residue subjected to chromatography (EtOAc) to give N-{6-[2-(4-Benzo[d]isothiazol-3-yl-piperazin-1-yl)-ethyl]-indan-1-yl}-acetamide (0.97 g, 2.30 mmole) with 100% purity @ 254 nm; LCMS (APCI) 421 [M+H]⁺. 1H NMR (400 MHz, CHLOROFORM-D) δ ppm 1.94 (s, 3 H) 2.65-2.71 (m, 2 H) 2.73-2.80 (m, 6 H) 2.81-2.87 (m, 2 H) 3.28 (ddd, J=16.04, 6.10, 5.92 Hz, 2 H) 3.57-3.62 (m, 4 H) 4.69-4.76 (m, 1 H) 5.70 (d, J=7.08 Hz, 1 H) 7.06 (d, J=7.81 Hz, 1 H) 7.11 (s, 1 H) 7.16 (d, J=7.56 Hz, 1 H) 7.35 (ddd, J=8.17, 7.08, 1.10 Hz, 1 H) 7.46 (ddd, J=8.11, 7.02, 1.22 Hz, 1 H) 7.81 (dt, J=8.05, 0.85 Hz, 1 H) 7.91 (dt, J=8.30, 0.98 Hz, 1 H).

### Example 4

### N-{5-[2-(4-BENZO[D]ISOTHIAZOL-3-YL-PIPERAZIN-1-YL)-ETHYL]-INDAN-2-YL}-BUTYRAMIDE

5-[2-(4-Benzo[d]isothiazol-3-yl-piperazin-1-yl)-ethyl]-indan-2-ylamine was diluted to 0.20 M with anhydrous dichloromethane, then delivered to an 8 mL vial via pipette (0.20 mmol). To the amine solution was added PS—N-Methylmorpholine resin (0.40 mmol). Butyryl chloride was diluted to 0.20 M with dichloromethane, and added at rt (0.40 mmol). The solution was shaken overnight at rt. Polyamine scavenging resin was added (0.5 mmol). The solution was shaken overnight at room temperature, then filtered into an 8 mL vial. The filtrate was evaluated by MS, then concentrated using an HT-12 GeneVac. Crude was purified by HPLC (30×100 mm ODS-A C(18) 5u column). N-{5-[2-(4-Benzo[d]isothiazol-3-yl-piperazin-1-yl)-ethyl]-indan-2-yl}-butyramide was isolated in 100% purity @ 254 nm, LCMS (APCI) 449 [M+H]⁺.

The amides of Examples 5-42 were synthesized in combinatorial library format following the steps outlined in example 1 on a 0.20 mmol scale using 5-[2-(4-Benzo[d]isothiazol-3-yl-piperazin-1-yl)-ethyl]-indan-2-ylamine with appropriate acid chloride starting materials and N-methylmorpholine on polystyrene resin. The crude products were purified by HPLC (30×100 mm ODS-A C(18) 5u column).

| Example | | |
|---|---|---|
| Number | Compound Name | Data |
| 5 | ISOXAZOLE-5-CARBOXYLIC ACID {5-[2-(4- | Isolated in 90 purity @ 254nm; |
| | BENZO[D]ISOTHIAZOL-3-YL-PIPERAZIN-1- | LCMS(APCI) |
| | YL)-ETHYL]-INDAN-2-YL}-AMIDE | 474[M+H]⁺ |
| 6 | THIOPHENE-2-CARBOXYLIC ACID {5-[2-(4- | Isolated in 100% purity @ |
| | BENZO[D]ISOTHIAZOL-3-YL-PIPERAZIN-1- | 254nm; LCMS(APCI) |
| | YL)-ETHYL]-INDAN-2-YL}-AMIDE | 489[M+H]⁺ |
| 7 | N-{5-[2-(4-BENZO[D]ISOTHIAZOL-3-YL- | Isolated in 100% purity @ |
| | PIPERAZIN-1-YL)-ETHYL]-INDAN-2-YL}-2- | 254nm; LCMS(APCI) |
| | THIOPHEN-2-YL-ACETAMIDE | 503[M+H]⁺ |
| 8 | N-{5-[2-(4-BENZO[D]ISOTHIAZOL-3-YL- | Isolated in 100% purity @ |
| | PIPERAZIN-1-YL)-ETHYL]-INDAN-2-YL}- | 254nm; LCMS(APCI) |
| | BENZAMIDE | 483[M+H]⁺ |
| 9 | N-{5-[2-(4-BENZO[D]ISOTHIAZOL-3-YL- | Isolated in 100% purity @ |
| | PIPERAZIN-1-YL)-ETHYL]-INDAN-2-YL}-4- | 254nm; LCMS(APCI) |
| | METHYL-BENZAMIDE | 497[M+H]⁺ |
| 10 | N-{5-[2-(4-BENZO[D]ISOTHIAZOL-3-YL- | Isolated in 100% purity @ |
| | PIPERAZIN-1-YL)-ETHYL]-INDAN-2-YL}-2- | 254nm; LCMS(APCI) |
| | PHENYL-ACETAMIDE | 497[M+H]⁺ |
| 11 | N-{5-[2-(4-BENZO[D]ISOTHIAZOL-3-YL- | Isolated in 100% purity @ |
| | PIPERAZIN-1-YL)-ETHYL]-INDAN-2-YL}-4- | 254nm; LCMS(APCI) |
| | FLUORO-BENZAMIDE | 501[M+H]⁺ |
| 12 | N-{5-[2-(4-BENZO[D]ISOTHIAZOL-3-YL- | Isolated in 98% purity @ |
| | PIPERAZIN-1-YL)-ETHYL]-INDAN-2-YL}-4- | 254nm; LCMS(APCI) |
| | CYANO-BENZAMIDE | 508[M+H]⁺ |
| 13 | N-{5-[2-(4-BENZO[D]ISOTHIAZOL-3-YL- | Isolated in 100% purity @ |
| | PIPERAZIN-1-YL)-ETHYL]-INDAN-2-YL}- | 254nm; LCMS(APCI) |
| | ISOBUTYRAMIDE | 449[M+H]⁺ |
| 14 | N-{5-[2-(4-BENZO[D]ISOTHIAZOL-3-YL- | Isolated in 98% purity @ |
| | PIPERAZIN-1-YL)-ETHYL]-INDAN-2-YL}-3- | 254nm; LCMS(APCI) |
| | PHENYL-PROPIONAMIDE | 511[M+H]⁺ |
| 15 | N-{5-[2-(4-BENZO[D]ISOTHIAZOL-3-YL- | Isolated in 100% purity @ |
| | PIPERAZIN-1-YL)-ETHYL]-INDAN-2-YL}-4- | 254nm; LCMS(APCI) |
| | METHOXY-BENZAMIDE | 513[M+H]⁺ |
| 16 | N-{5-[2-(4-BENZO[D]ISOTHIAZOL-3-YL- | Isolated in 100% purity @ |
| | PIPERAZIN-1-YL)-ETHYL]-INDAN-2-YL}-4- | 254nm; LCMS(APCI) |
| | CHLORO-BENZAMIDE | 518[M+H]⁺ |
| 17 | N-{5-[2-(4-BENZO[D]ISOTHIAZOL-3-YL- | Isolated in 99% purity @ |
| | PIPERAZIN-1-YL)-ETHYL]-INDAN-2-YL}-2- | 254nm; LCMS(APCI) |
| | (3,4-DIMETHOXY-PHENYL)-ACETAMIDE | 557[M+H]⁺ |
| 18 | N-{5-[2-(4-BENZO[D]ISOTHIAZOL-3-YL- | Isolated in 100% purity @ |
| | PIPERAZIN-1-YL)-ETHYL]-INDAN-2-YL}-3-(3- | 254nm; LCMS(APCI) |
| | METHYL-PYRAZOL-1-YL)-PROPIONAMIDE | 515[M+H]⁺ |
| 19 | 4-METHOXY-CYCLOHEXANECARBOXYLIC | Isolated in 100% purity @ |
| | ACID {5-[2-(4-BENZO[D]ISOTHIAZOL-3-YL- | 254nm; LCMS(APCI) |
| | PIPERAZIN-1-YL)-ETHYL]-INDAN-2-YL}- | 519[M+H]⁺ |
| | AMIDE | |
| 20 | N-{5-[2-(4-BENZO[D]ISOTHIAZOL-3-YL- | Isolated in 100% purity @ |
| | PIPERAZIN-1-YL)-ETHYL]-INDAN-2-YL}- | 254nm; LCMS(APCI) |
| | PROPIONAMIDE | 435[M+H]⁺ |
| 21 | CYCLOPROPANECARBOXYLIC ACID {5-[2- | Isolated in 100% purity @ |
| | (4-BENZO[D]ISOTHIAZOL-3-YL-PIPERAZIN- | 254nm; LCMS(APCI) |
| | 1-YL)-ETHYL]-INDAN-2-YL}-AMIDE | 447[M+H]⁺ |
| 22 | CYCLOBUTANECARBOXYLIC ACID {5-[2-(4- | Isolated in 100% purity @ |
| | BENZO[D]ISOTHIAZOL-3-YL-PIPERAZIN-1- | 254nm; LCMS(APCI) |
| | YL)-ETHYL]-INDAN-2-YL}-AMIDE | 461[M+H]⁺ |
| 23 | N-{5-[2-(4-BENZO[D]ISOTHIAZOL-3-YL- | Isolated in 100% purity @ |
| | PIPERAZIN-1-YL)-ETHYL]-INDAN-2-YL}-2- | 254nm; LCMS(APCI) |
| | METHOXY-ACETAMIDE | 451[M+H]⁺ |
| 24 | 1-METHYL-CYCLOPROPANECARBOXYLIC | Isolated in 100% purity @ |
| | ACID {5-[2-(4-BENZO[D]ISOTHIAZOL-3-YL- | 254nm; LCMS(APCI) |
| | PIPERAZIN-1-YL)-ETHYL]-INDAN-2-YL}- | 461[M+H]⁺ |
| | AMIDE | |
| 25 | 3-ACETYLAMINO-N-{5-[2-(4- | Isolated in 99% purity @ |
| | BENZO[D]ISOTHIAZOL-3-YL-PIPERAZIN-1- | 254nm; LCMS(APCI) |
| | YL)-ETHYL]-INDAN-2-YL}-PROPIONAMIDE | 492[M+H]⁺ |
| 26 | 2,2,3,3-TETRAMETHYL- | Isolated in 100% purity @ |
| | CYCLOPROPANECARBOXYLIC ACID {5-[2- | 254nm; LCMS(APCI) |
| | (4-BENZO[D]ISOTHIAZOL-3-YL-PIPERAZIN- | 503[M+H]⁺ |
| | 1-YL)-ETHYL]-INDAN-2-YL}-AMIDE | |
| 27 | 1H-PYRROLE-2-CARBOXYLIC ACID {5-[2- | Isolated in 96% purity @ |
| | (4-BENZO[D]ISOTHIAZOL-3-YL-PIPERAZIN- | 254nm; LCMS(APCI) |
| | 1-YL)-ETHYL]-INDAN-2-YL}-AMIDE | 472[M+H]⁺ |
| 28 | 1-METHYL-1H-PYRROLE-2-CARBOXYLIC | Isolated 97% purity @ 254 |
| | ACID {5-[2-(4-BENZO[D]ISOTHIAZOL-3-YL- | nm; LCMS(APCI) |
| | PIPERAZIN-1-YL)-ETHYL]-INDAN-2-YL}- | 486[M+H]⁺ |
| | AMIDE | |
| 29 | 1-METHYL-1H-IMIDAZOLE-2-CARBOXYLIC | Isolated in 100% purity @ |
| | ACID {5-[2-(4-BENZO[D]ISOTHIAZOL-3-YL- | 254nm; LCMS(APCI) |
| | PIPERAZIN-1-YL)-ETHYL]-INDAN-2-YL}- | 487[M+H]⁺ |
| | AMIDE | |
| 30 | 2,4-DIMETHYL-THIAZOLE-5-CARBOXYLIC | Isolated in 100% purity @ |
| | ACID {5-[2-(4-BENZO[D]ISOTHIAZOL-3-YL- | 254nm; LCMS(APCI) |
| | PIPERAZIN-1-YL)-ETHYL]-INDAN-2-YL}- | 518[M+H]⁺ |
| | AMIDE | |
| 31 | N-{5-[2-(4-BENZO[D]ISOTHIAZOL-3-YL- | Isolated in 99% purity @ |
| | PIPERAZIN-1-YL)-ETHYL]-INDAN-2-YL}-2- | 254nm; LCMS(APCI) |
| | PHENOXY-ACETAMIDE | 513[M+H]⁺ |
| 32 | N-{5-[2-(4-BENZO[D]ISOTHIAZOL-3-YL- | Isolated in 99% purity @ |
| | PIPERAZIN-1-YL)-ETHYL]-INDAN-2-YL}-2-(4- | 254nm; LCMS(APCI) |
| | FLUORO-PHENYL)-ACETAMIDE | 515[M+H]⁺ |
| 33 | N-{5-[2-(4-BENZO[D]ISOTHIAZOL-3-YL- | Isolated in 100% purity @ |
| | PIPERAZIN-1-YL)-ETHYL]-INDAN-2-YL}-2,2- | 254nm; LCMS(APCI) |
| | DIMETHYL-PROPIONAMIDE | 463[M+H]⁺ |
| 34 | N-{5-[2-(4-BENZO[D]ISOTHIAZOL-3-YL- | Isolated in 99% purity @ |
| | PIPERAZIN-1-YL)-ETHYL]-INDAN-2YL}-3- | 254nm; LCMS(APCI) |
| | PYRIDIN-3-YL-PROPIONAMIDE | 512[M+H]⁺ |
| 35 | N-{5-[2-(4-BENZO[D]ISOTHIAZOL-3-YL- | Isolated in 99% purity @ |
| | PIPERAZIN-1-YL)-ETHYL]-INDAN-2-YL}-2- | 254nm; LCMS(APCI) |
| | ETHYLSULFANYL-ACETAMIDE | 512[M+H]⁺ |
| 36 | PYRAZINE-2-CARBOXYLIC ACID {5-[2-(4- | Isolated in 100% purity @ |
| | BENZO[D]ISOTHIAZOL-3-YL-PIPERAZIN-1- | 254nm; LCMS(APCI) |
| | YL)-ETHYL]-INDAN-2-YL}-AMIDE | 485[M+H]⁺ |
| 37 | N-{5-[2-(4-BENZO[D]ISOTHIAZOL-3-YL- | Isolated in 98% purity @ |
| | PIPERAZIN-1-YL)-ETHYL]-INDAN-2-YL}-3- | 254nm; LCMS(APCI) |
| | HYDROXY-2-METHYL-BENZAMIDE | 513[M+H]⁺ |
| 38 | CYCLOPENTANECARBOXYLIC ACID {5-[2- | Isolated in 100% purity @ |
| | (4-BENZO[D]ISOTHIAZOL-3-YL-PIPERAZIN- | 254nm; LCMS(APCI) |
| | 1-YL)-ETHYL]-INDAN-2-YL}-AMIDE | 475[M+H]⁺ |
| 39 | N-{5-[2-(4-BENZO[D]ISOTHIAZOL-3-YL- | Isolated in 100% purity @ |
| | PIPERAZIN-1-YL)-ETHYL]-INDAN-2-YL}-3- | 254nm; LCMS(APCI) |
| | METHYL-BUTYRAMIDE | 463[M+H]⁺ |
| 40 | N-{5-[2-(4-BENZO[D]ISOTHIAZOL-3-YL- | Isolated in 100% purity @ |
| | PIPERAZIN-1-YL)-ETHYL]-INDAN-2-YL}-3,3- | 254nm; LCMS(APCI) |
| | DIMETHYL-BUTYRAMIDE | 477[M+H]⁺ |
| 41 | CYCLOHEXANECARBOXYLIC ACID {5-[2-(4- | Isolated in 100% purity @ |
| | BENZO[D]ISOTHIAZOL-3-YL-PIPERAZIN-1- | 254nm; LCMS(APCI) |
| | YL)-ETHYL]-INDAN-2-YL}-AMIDE | 489[M+H]⁺ |
| 42 | FURAN-2-CARBOXYLIC ACID {5-[2-(4- | Isolated in 99% purity @ |
| | BENZO[D]ISOTHIAZOL-3-YL-PIPERAZIN-1- | 254nm; LCMS(APCI) |
| | YL)-ETHYL]-INDAN-2-YL}-AMIDE | 473[M+H]⁺ |

### Preparation 4

### 2,2,2-TRIFLUORO-N-INDAN-1-YL-ACETAMIDE

Starting with Indan-1-ylamine (20.88 g, 156.77 mmole), trifluoroacetic anhydride (33.21 ml, 235.15 mmole) and triethyl amine (2.0 eq) and following the procedure as outlined in Preparation 1, 33.75 g of 2,2,2-Trifluoro-N-indan-1-yl-acetamide was isolated in 94% yield. 1H NMR (400 MHz, CHLOROFORM-D) δ ppm 1.91 (m, 1 H) 2.65 (m, 1 H) 2.92 (ddd, J=16.00, 7.94, 7.82 Hz, 1 H) 3.04 (ddd, J=16.12, 8.79, 4.40 Hz, 1 H) 5.49 (q, J=7.57 Hz, 1 H) 6.42 (s, 1 H) 7.27 (m, 4 H).

### Preparation 5

### N-[6-(2-CHLORO-ACETYL)-INDAN-1-YL]-2,2,2-TRIFLUORO-ACETAMIDE

To a solution of 2,2,2-Trifluoro-N-indan-1-yl-acetamide (33.45 g, 145.94 mmole) in DCM (300 ml) was added AlCl₃ (58.38 g, 437.82 mmole) in one portion followed by addition of chloroacetyl chloride (21.5 ml, 218.91 mmole).The reaction was heated to 40° C. for 1.5 h then cooled to <rt. The reaction was poured into iced water and diluted with DCM. The layers were separated and the aqueous washed with DCM (3×200 ml). The organics were washed with water (250 ml) brine (250 ml), dried (MgSO₄) and concentrated to a solid mass. Dissolved in hot IPA (500 ml) and recrystallized overnight. N-[6-(2-Chloro-acetyl)-indan-1-yl]-2,2,2-trifluoro-acetamide was isolated by filtration as a tan cotton like solid and dried in vacuo at 50° C. overnight. 1H NMR (400 MHz, CHLOROFORM-D) δ ppm 2.00 (m, 1 H) 2.72 (m, 1 H) 2.98 (m, 1 H) 3.09 (m, 1 H) 3.13 (m, 1 H) 4.62 (s, 2H) 5.53 (dd, J=7.82, 16.37 Hz, 1 H, NOE with singlet at 7.81 ppm) 6.65 (s, 1 H, NOE with singlet at 7.81 ppm) 7.39 (d, J=7.81 Hz, 1 H) 7.81 (s, 1 H, NOE with singlet at 6.65 ppm and dd at 5.53 ppm) 7.86 (dd, J=8.06, 1.47 Hz, 2 H). The filtrate from above was chromatographed (20% EtOAc/Hex) to give two compounds of R_{f} 0.2 and 0.17. The compound with R_{f} of 0.2 was determined to be regioisomer N-[4-(2-Chloro-acetyl)-indan-1-yl]-2,2,2-trifluoro-acetamide. 1H NMR (400 MHz, CHLOROFORM-D) δ ppm 1.94 (m, 1 H) 2.68 (m, 1 H) 3.23 (ddd, J18.13, 8.00, 7.82 Hz, 1 H) 3.46 (ddd, J=18.19, 8.91, 4.40 Hz, 1 H) 4.68 (d, J=1.47 Hz, 2 H) 5.53 (q, J=7.90 Hz, 1 H) 6.45 (s, 1 H) 7.39 (t, J=7.69 Hz, 1 H) 7.54 (d, J=7.57 Hz, 1 H) 7.76 (d, J=7.81 Hz, 1 H). The compound with R_{f} of 0.17 was recrystallized from EtOAc/Hex to give N-[5-(2-Chloro-acetyl)-indan-1-yl]-2,2,2-trifluoro-acetamide. 1H NMR (400 MHz, CHLOROFORM-D) δ ppm 1.96 (m, 1 H) 2.73 (m, 1 H) 2.98 (m, 1 H) 3.08 (m, 1 H) 4.67 (s, 2 H) 5.55 (q, J=8.06 Hz, 1 H) 6.50 (s, 1 H) 7.39 (d, J=8.06 Hz, 1 H) 7.82 (d, J=7.81 Hz, 1 H) 7.85 (s, 1 H)

### Preparation 6

### N-[6-(2-CHLORO-ETHYL)-INDAN-1-YL1-2,2,2-TRIFLUORO-ACETAMIDE

Starting with N-[6-(2-Chloro-acetyl)-indan-1-yl]-2,2,2-trifluoro-acetamide (1.00 g, 3.275 mmole) and following the procedure outlined in Preparation 3, (0.67 g, 2.29 mmole) N-[6-(2-Chloro-ethyl)-indan-1-yl]-2,2,2-trifluoro-acetamide was isolated. 1H NMR (400 MHz, CHLOROFORM-D) δ ppm 1.91 (m, 1 H) 2.66 (m, 1 H) 2.89 (ddd, J=15.94, 7.88, 7.69 Hz, 1 H) 3.00 (m, 1 H) 3.05 (t, J=7.20 Hz, 2 H) 3.69 (t, J=7.33 Hz, 2 H) 5.48 (q, J=7.57 Hz, 1 H) 6.39 (s, 1 H) 7.13 (s, 1 H) 7.15 (d, J=7.81 Hz, 1 H) 7.22 (m, 1 H)

### Example 43

### N-{6-[2-(4-BENZO[D]ISOTHIAZOL-3-YL-PIPERAZIN-1-YL)-ETHYL]-INDAN-1-YL}-2,2,2-TRIFLUORO-ACETAMIDE

Starting with N-[6-(2-Chloro-ethyl)-indan-1-yl]-2,2,2-trifluoro-acetamide (6.35 g, 21.76 mmole), and 3-Piperazin-1-yl-benzo[d]isothiazole (11.13 g, 43.52 mmole) and following the procedure outlined in Example 1, (6.31 g, 13.30 mmole) of N-{6-[2-(4-Benzo[d]isothiazol-3-yl-piperazin-1-yl)-ethyl]-indan-1-yl}-2,2,2-trifluoro-acetamide was prepared with 100% purity @ 254 nm; LCMS (APCI) 475 [M+H]⁺. 1H NMR (400 MHz, CHLOROFORM-D) δ ppm 1.91 (m, 1 H) 2.66 (m, 3 H) 2.76 (m, 4 H) 2.88 (m, 3 H) 3.01 (ddd, J=16.30, 8.73, 4.52 Hz, 1 H) 3.59 (m, 4 H) 5.49 (q, J=7.82 Hz, 1 H) 6.41 (d, J=8.55 Hz, 1 H) 7.18 (m, 3 H) 7.35 (ddd, J=8.18, 7.08, 0.85 Hz, 1 H) 7.46 (ddd, J=8.06, 6.96, 1.10 Hz, 1 H) 7.81 (d, J=8.30 Hz, 1 H) 7.91 (d, J=8.30 Hz, 1 H).

### Example 44

### 6-[2-(4-BENZO[D]ISOTHIAZOL-3-YL-PIPERAZIN-1-YL)-ETHYL]-INDAN-1-YLAMINE

To a solution of N-{6-[2-(4-Benzo[d]isothiazol-3-yl-piperazin-1-yl)-ethyl]-indan-1-yl}-2,2,2-trifluoro-acetamide (4.35 g, 9.17 mmole) in MeOH/H₂O (3:1) was added K₂CO₃ (6.34 g, 45.85 mmole). The whole was subjected to microwave irradiation and heated to 100° C. for 15 min using the CEM MARS-5 microwave reactor. Upon cooling, the reaction was concentrated and diluted with EtOAc (200 ml) and water (100 ml). The layers were separated and the aqueous extracted with EtOAc (100 ml). The combined organics were washed with water (2×50 ml), brine (50 ml), and dried (MgSO₄) and concentrated to dryness. The residue was taken up in 1,4-dioxane/Et₂O (2:1) followed by treatment with 1N HCl in Et₂O. The precipitate was collected by filtration as a pale yellow solid and dried at 50° C. under hi-vac to give 6-[2-(4-Benzo[d]isothiazol-3-yl-piperazin-1-yl)-ethyl]-indan-1-ylamine (3.371 g, 8.90 mmole) with 100% purity @ 254 nm; LCMS (APCI) 379 [M+H]⁺. 1H NMR (400 MHz, CHLOROFORM-D) δ ppm 2.16 (m, 1 H) 2.51 (m, 1 H) 2.91 (m, 6 H) 3.12 (ddd, J=16.12, 8.43, 5.25 Hz, 1 H) 3.68 (s, 4 H) 4.65 (t, J=6.59 Hz, 1 H) 7.05 (d, J=7.82 Hz, 1 H) 7.11 (m, 1 H) 7.33 (ddd, J=8.06, 7.08, 0.98 Hz, 1 H) 7.44 (td, J=7.57, 0.98 Hz, 1 H) 7.63 (s, 1 H) 7.77 (d, J=8.06 Hz, 1 H) 7.84 (d, J=8.30 Hz, 1 H)

### Example 45

### N-{6-[2-(4-BENZO[D]ISOTHIAZOL-3-YL-PIPERAZIN-1-YL)-ETHYL]-INDAN-1-YL}-ACETAMIDE

To a solution of 6-[2-(4-Benzo[d]isothiazol-3-yl-piperazin-1-yl)-ethyl]-indan-1-ylamine (1.00 g, 2.41 mmole) and Et₃N (1.08 ml, 7.23 mmole) in dry THF (10 ml) was added acetyl chloride (0.26 ml, 3.61 mmole). The reaction was stirred at rt for 1 h. then quenched with water (25 ml). The reaction was diluted with EtOAc (50 ml) and the layers separated. The organics were dried (MgSO₄), concentrated and the residue subjected to chromatography (EtOAc) to give N-{6-[2-(4-Benzo[d]isothiazol-3-yl-piperazin-1-yl)-ethyl]-indan-1-yl}-acetamide (0.97 g, 2.30 mmole) with 100% purity @ 254 nm; LCMS (APCI) 421 [M+H]⁺. 1H NMR (400 MHz, CHLOROFORM-D) δ ppm 1.71-1.83 (m, 1 H) 2.04 (s, 3 H) 2.52-2.63 (m, 1 H) 2.63-2.70 (m, 2 H) 2.70-2.78 (m, 4H) 2.78-2.87 (m, 3H) 2.88-2.97 (m, 1 H) 3.59-3.63 (m, 4 H) 5.46 (dd, J=7.73, 16.80 Hz, 1 H) 5.65 (d, J=8.79 Hz, 1 H) 7.11 (d, J=7.81 Hz, 1 H) 7.16 (m, 2 H) 7.35 (ddd, J=8.12, 7.02, 0.98 Hz, 1 H) 7.46 (ddd, J=8.06, 7.08, 0.98 Hz, 1 H) 7.81 (d, J=8.06 Hz, 1 H) 7.91 (d, J=8.06 Hz, 1 H).

Examples 46-58 were synthesized in a parallel format following the steps outlined in example 1 on a 0.12 mmole scale using Benzo[d]isothiazol-3-yl-piperazin-1-yl)-ethyl]-indan-1-ylamine hydrochloride salt (0.259 g, 0.624 mmole) with appropriate acid chloride (1.5 eq) starting materials and Et₃N (2 eq) in THF (5 ml). The crude products were purified by chromatography (50% EtOAc/Hex).

| Example | | |
|---|---|---|
| Number | Compound Name | Data |
| 46 | N-{6-[2-(4- | 100% purity @ 254nm; LCMS(APCI) |
| | BENZO[D]ISOTHIAZOL-3-YL- | 435[M+H]⁺. 1H NMR(400MHz, |
| | PIPERAZIN-1-YL)-ETHYL]- | CHLOROFORM-D) δ ppm 1.19(t, |
| | INDAN-1-YL}-PROPIONAMIDE | J=7.57Hz, 3H) 1.76(ddd, J=15.75, |
| | | 12.94, 8.43Hz, 1H) 2.25(t, J=7.57Hz, |
| | | 2H) 2.54-2.64(m, 1H) 2.65-2.88(m, |
| | | 9H) 2.93(ddd, J=16.06, 8.85, 3.91Hz, 1H) |
| | | 3.52-3.69(s, 4H) 5.47(dd, J=7.35, |
| | | 17.35Hz, 1H) 5.60(d, J=8.55Hz, 1H) |
| | | 7.11(d, J=7.75Hz, 1H) 7.12(s, 1H) |
| | | 7.16(d, J=7.75Hz, 1H) 7.34(ddd, |
| | | J=8.18, 7.08, 0.85Hz, 1H) 7.45(ddd, |
| | | J=8.06, 7.08, 0.98Hz, 1H) 7.80(d, |
| | | J=8.06Hz, 1H) 7.89(d, J=8.30Hz, 1H). |
| 47 | N-{6-[2-(4- | 100% purity @ 254nm; LCMS(APCI) |
| | BENZO[D]ISOTHIAZOL-3-YL- | 449[M+H]⁺. 1H NMR(400MHz, |
| | PIPERAZIN-1-YL)-ETHYL]- | CHLOROFORM-D) δ ppm 0.96(t, |
| | INDAN-1-YL}-BUTYRAMIDE | J=7.45Hz, 3H) 1.73(m, 3H) 2.18(t, |
| | | J=7.15Hz, 2H) 2.53-2.63(m, 1H) 2.64-2.71(m, |
| | | 2H) 2.73-2.88(m, 6H) 2.92(ddd, |
| | | J=15.87, 8.79, 3.66Hz, 1H) 3.54-3.65(m, |
| | | 4H) 5.47(dd, J=15.90, 7.73Hz, |
| | | 1H) 5.61(d, J=8.55Hz, 1H) 7.09(d, |
| | | J=7.60Hz, 1H) 7.11(s, 1H) 7.16(d, |
| | | J=7.60Hz, 1H) 7.34(t, 8.06Hz, 1H) |
| | | 7.45(t, J=8.06Hz, 1H) 7.79(d, J=8.06Hz, |
| | | 2H) 7.89(d, J=8.06Hz, 2H). |
| 48 | CYCLOPROPANECARBOXYLIC | 100% purity @ 254nm; LCMS(APCI) |
| | ACID {6-[2-(4- | 447[M+H]⁺. 1H NMR(400MHz, |
| | BENZO[D]ISOTHIAZOL-3-YL- | CHLOROFORM-D) δppm 0.71-0.78(m, |
| | PIPERAZIN-1-YL)-ETHYL]- | 2H) 0.99-1.06(m, 2H) 1.27-1.36(m, 1H) |
| | INDAN-1-YL}-AMIDE | 1.79(ddd, J=15.87, 12.94, 8.55Hz, 1H) |
| | | 2.53-2.63(m, 1) 2.63-2.69(m, 2H) 2.72-2.78(m, |
| | | 4H) 2.78-2.87(m, 3H) 2.94(ddd, |
| | | J=15.69, 8.85, 4.03Hz, 1H) 3.54-3.61(m, |
| | | 4H) 5.48(dd, J=16.75, 7.57Hz, |
| | | 1H) 5.78(d, J=8.79Hz, 1H) 7.10(d, |
| | | J=8.79Hz, 1H) 7.16(m, 2H) 7.34(t, |
| | | J=8.18Hz, 1H) 7.45(t, J=8.18Hz, 1H) |
| | | 7.79(d, J=8.06Hz, 1H) 7.89(d, J=8.06Hz, |
| | | 1H). |
| 49 | N-{6-[2-(4- | 100% purity @ 254nm; LCMS(APCI) |
| | BENZO[D]ISOTHIAZOL-3-YL- | 449[M+H]⁺. 1H NMR(400MHz, |
| | PIPERAZIN-1-YL)-ETHYL]- | CHLOROFORM-D) δ ppm 1.19(dd, |
| | INDAN-1-YL}- | rotomers, J=7.94, 6.96Hz, 6H) |
| | ISOBUTYRAMIDE | 1.74(ddd, J=16.18, 12.88, 8.55Hz, 1H) |
| | | 2.36(heptet, J=6.85Hz, 1H) 2.54-2.71(m, |
| | | 3H) 2.71-2.79(m, 4H) 2.79-2.88(m, 3H) |
| | | 2.91(ddd, J=16.00, 8.79, 3.54Hz, 1H) |
| | | 3.54-3.65(m, 4H) 5.47(dd, J=16.51, |
| | | 7.82Hz, 1H) 5.59(d, J=8.30Hz, 1H) |
| | | 7.07-7.12(m, 2H) 7.16(d, J- 8.10Hz, |
| | | 1H) 7.34(t, J=8.06Hz, 1H) 7.45(t, |
| | | J=8.18Hz, 1H) 7.79(d, J=8.06Hz, 1H) |
| | | 7.89(d, J=8.30Hz, 1H). |
| 50 | N-{6-[2-(4- | 100% purity @ 254nm; LCMS(APCI) |
| | BENZO[D]ISOTHIAZOL-3-YL- | 517[M+H]⁺. 1H NMR(400MHz, |
| | PIPERAZIN-1-YL)-ETHYL]- | CHLOROFORM-D) δ ppm 1.90(ddd, |
| | INDAN-1-YL}-4-CHLORO- | J=15.75, 12.82, 8.30Hz, 1H) 2.69(m, |
| | BENZAMIDE | 7H) 2.80-2.93(m, 3H) 2.99(ddd, |
| | | J=15.94, 8.73, 3.91Hz, 1H) 3.56(m, |
| | | 4H) 5.66(dd, J=15.79, 7.57Hz, 1H) |
| | | 6.25(d, J=8.30Hz, 1H) 7.13(dd, J=7.45, |
| | | 1.59Hz, 1H) 7.20(m, 2H) 7.33(ddd, |
| | | J=8.12, 7.02, 0.98Hz, 1H) 7.38(m, 1H) |
| | | 7.40(m, 1H) 7.44(ddd, J=8.12, 7.02, |
| | | 1.22Hz, 1H) 7.71(m, 1H) 7.73(m, 1H) |
| | | 7.79(d, J=8.06Hz, 1H) 7.88(d, |
| | | J=8.06Hz, 1H). |
| 51 | N-{6-[2-(4- | 100% purity @ 254nm; LCMS(APCI) |
| | BENZO[D]ISOTHIAZOL-3-YL- | 497[M+H]⁺. 1H NMR(400MHz, |
| | PIPERAZIN-1-YL)-ETHYL]- | CHLOROFORM-D) δ ppm 1.89(ddd, |
| | INDAN-1-YL}-4-METHYL- | J=16.06, 12.88, 8.43Hz, 1H) 2.36(s, |
| | BENZAMIDE | 3H) 2.61-2.77(m, 7H) 2.79-2.82(m, 3H) |
| | | 2.98(ddd, J=15.81, 8.85, 3.66Hz, 1H) |
| | | 3.51-3.60(m, 4H) 5.67(dd, J=16.41, |
| | | 7.73Hz, 1H) 6.27(d, J=8.30Hz, 1H) |
| | | 7.12(d, J=7.81Hz, 1H) 7.17-7.23(m, |
| | | 4H) 7.32(t, J=7.65Hz, 1H) 7.44(t, J=7.65Hz, |
| | | 1H) 7.68(d, J=8.06Hz, 2H) |
| | | 7.79(d, J=8.06Hz, 1H) 7.88(d, J=8.30Hz, |
| | | 1H). |
| 52 | N-{6-[2-(4- | 100% purity @ 254nm; LCMS(APCI) |
| | BENZO[D]ISOTHIAZOL-3-YL- | 483[M+H]⁺. 1H NMR(400MHz, |
| | PIPERAZIN-1-YL)-ETHYL]- | CHLOROFORM-D) δ ppm 1.91(ddd, |
| | INDAN-1-YL}-BENZAMIDE | J=16.00, 12.82, 8.55Hz, 1H) 2.62-2.79(m, |
| | | 7H) 2.80-2.93(m, 3H) 3.00(ddd, J=16.12, |
| | | 8.79, 3.66Hz, 1H) 3.50-3.63(m, |
| | | 4H) 5.68(dd, J=16.19, 7.65Hz, 1H) |
| | | 6.32(d, J=8.30Hz, 1H) 7.13(d, J=7.82Hz, |
| | | 1H) 7.19(d, J=4.40Hz, 2H) 7.33(ddd, |
| | | J=8.12, 7.02, 0.98Hz, 1H) 7.38-7.51(m, |
| | | 4H) 7.76-7.81(m, 3H) 7.88(d, |
| | | J=8.06Hz, 1H). |
| 53 | N-{6-[2-(4- | 100% purity @ 254nm; LCMS(APCI) |
| | BENZO[D]ISOTHIAZOL-3-YL- | 501[M+H]⁺. 1H NMR(400MHz, |
| | PIPERAZIN-1-YL)-ETHYL]- | CHLOROFORM-D) δ ppm 1.90(ddd, |
| | INDAN-1-YL}-4-FLUORO- | J=15.81, 12.88, 8.43Hz, 1H) 2.61-2.78(m, |
| | BENZAMIDE | 7H) 2.80-2.93(m, 3H) 2.99(ddd, |
| | | J=15.75, 8.67, 3.91Hz, 1H) 3.51-3.60(m, |
| | | 4H) 5.65(dd, J=16.20, 7.57Hz, 1H) |
| | | 6.26(d, J=8.30Hz, 1H) 7.05-7.15(m, |
| | | 3H) 7.16-7.22(m, 2H) 7.33(ddd, |
| | | J=8.02, 8.19, 1.10Hz, 1H) 7.44(ddd, |
| | | 8.02, 8.10 0.981H) 7.76-7.82(m, |
| | | 3H) 7.88(d, J=8.06Hz, 1H). |
| 54 | N-{6-[2-(4- | 100% purity @ 254nm; LCMS(APCI) |
| | BENZO[D]ISOTHIAZOL-3-YL- | 497[M+H]⁺. 1H NMR(400MHz, |
| | PIPERAZIN-1-YL)-ETHYL]- | CHLOROFORM-D) δ ppm 1.55-1.69(m, |
| | INDAN-1-YL}-2-PHENYL- | 1H) 2.51-2.68(m, 3H) 2.70-2.90(m, |
| | ACETAMIDE | 8H) 3.55-3.67(m, 6H) 5.45(dd, J=17.28, |
| | | 8.14Hz, 1H) 5.54(d, 8.70Hz, 1H) 6.95(s, |
| | | 1H) 7.06(d, J=7.68Hz, 1H) 7.11(d, |
| | | J=7.168Hz, 1H) 7.24-7.29(m, 3H) 7.30-7.37(m, |
| | | 3H) 7.46(td, J=7.51, 1.10Hz, |
| | | 1H) 7.80(d, J=7.99Hz, 1H) 7.91(d, J=7.99Hz, |
| | | 1H). |
| 55 | N-{6-[2-(4- | 100% purity @ 254nm; LCMS(APCI) |
| | BENZO[D]ISOTHIAZOL-3-YL- | 511[M+H]⁺. 1H NMR(400MHz, |
| | PIPERAZIN-1-YL)-ETHYL]- | CHLOROFORM-D) δ ppm 1.53-1.70(m, |
| | INDAN-1-YL}-3-PHENYL- | 2H) 2.47-2.58(m, 3H) 2.59-2.68(m, |
| | PROPIONAMIDE | 2H) 2.71-2.90(m, 7H) 2.95-3.06(m, 2H) |
| | | 3.54-3.63(m, 4H) 5.37-5.51(m, 2H) |
| | | 6.94(s, 1H) 7.07(d, 6.86Hz, 1H) 7.13(d, |
| | | J=6.86Hz, 1H) 7.17-7.23(m, 3H) |
| | | 7.25-7.31(m, 2H) 7.34(td, J=7.57, 0.98Hz, |
| | | 1H), 7.45(td, J=7.57, 0.98Hz, 1H) |
| | | 7.79(d, J=8.37Hz, 1H), 7.90(d, J=8.37Hz, |
| | | 1H). |
| 56 | N-{6-[2-(4- | 100% purity @ 254nm; LCMS(APCI) |
| | BENZO[D]ISOTHIAZOL-3-YL- | 473[M+H]⁺. 1H NMR(400MHz, |
| | PIPERAZIN-1-YL)-ETHYL]- | CHLOROFORM-D) δ ppm 1.90(ddd, |
| | INDAN-1-YL}-2-FURAN-3-YL- | J=16.00, 12.94, 8.43Hz, 1H) 2.60-2.71(m, |
| | ACETAMIDE | 3H) 2.74(s, 4H) 2.79-2.92(m, 3H) |
| | | 2.99(ddd, J=15.94, 8.85, 3.79Hz, 1H) |
| | | 3.50-3.62(m, 4H) 5.63(dd, J=17.05, |
| | | 7.98Hz, 1H) 6.48(dd, J=3.42, 1.71Hz, |
| | | 1H) 6.54(d, J=8.55Hz, 1H) 7.10-7.16(m, |
| | | 2H) 7.17-7.21(m, 2H) 7.33(td, |
| | | J=7.57, 0.98Hz, 1H) 7.39(dd, J=1.83, |
| | | 0.85Hz, 1H) 7.44(td, J=7.57, 0.98Hz, |
| | | 1H) 7.79(d, J=8.06Hz, 1H) 7.88(d, |
| | | J=8.30Hz, 1H). |
| 57 | N-{6-[2-(4- | 100% purity @ 254nm; LCMS(APCI) |
| | BENZO[D]ISOTHIAZOL-3-YL- | 485[M+H]⁺. 1H NMR(400MHz, |
| | PIPERAZIN-1-YL)-ETHYL]- | CHLOROFORM-D) δ ppm 1.88-2.00(m, |
| | INDAN-1-YL}-NICOTINAMIDE | 1H) 2.64-2.76(m, 4H) 2.77(s, 3H) |
| | | 2.83-2.94(m, 3H) 2.95-3.06(m, 1H) |
| | | 3.59(s, 4H) 5.68(q, J=7.73Hz, 1H) |
| | | 6.36(d, J=7.82Hz, 1H) 7.14(dd, |
| | | J=7.45, 1.59Hz, 1H) 7.19-7.22(m, 2H) |
| | | 7.30-7.40(m, 2H) 7.45(ddd, J=8.06, |
| | | 6.96, 1.10Hz, 1H) 7.79(d, J=8.06Hz, 1H) |
| | | 7.87(d, J=8.30Hz, 1H) 8.11-8.16(m, |
| | | 1H) 8.71(dd, J=4.88, 1.71Hz, 1H) |
| | | 8.97(dd, J=2.32, 0.85Hz, 1H). |
| 58 | ISOXAZOLE-5-CARBOXYLIC | 100% purity @ 254nm; LCMS(APCI) |
| | ACID {6-[2-(4- | 474[M+H]⁺. 1H NMR(400MHz, |
| | BENZO[D]ISOTHIAZOL-3-YL- | CHLOROFORM-D) δ ppm 1.90-1.99(m, |
| | PIPERAZIN-1-YL)-ETHYL]- | 1H) 2.63-2.71(m, 4H) 2.73-2.78(m, |
| | INDAN-1-YL}-AMIDE | 4H) 2.82-2.91(m, 3H) 3.55-3.59(m, 4H) |
| | | 5.62(q, J=7.90Hz, 1H) 6.77(d, J=8.30Hz, |
| | | 1H) 6.96(d, J=1.71Hz, 1H) 7.13-7.21(m, |
| | | 3H) 7.33(td, J=7.57, 0.98Hz, |
| | | 1H) 7.44(td, J=7.57, 0.98Hz, 1H) 7.79(d, |
| | | J=8.06Hz, 1H) 7.88(d, J=8.30Hz, |
| | | 1H) 8.32(d, J=1.95Hz, 1H). |

### Preparation 7

### 5-(2-CHLORO-ETHYL)-INDAN-2-YL-AMMONIUM(L)(+)TARTRATE SALT

(±)2-indanamine (4.4 g, 22.5 mmol) and L-(+)-tartaric acid (3.4 g, 22.5 mmol) was dissolved in a refluxing mixture of 50 mL ethanol and 10 mL water. After 6 hrs stirring, the mixture was allowed to cool to rt, the obtained precipitate was collected, rinsed with ethanol/water(5/1) and dried. The crystalline salt was recrystallized twice. First from 50 mL ethanol/15 mL water and second with 50 mL ethanol and 20 mL water. 5-(2-chloro-ethyl)-indan-2-yl-ammonium (L)(+)Tartrate salt (0.94 g) was isolated with an e.e. of 98% for the amine. 1H NMR (200 MHz, D6-DMSO) δ 2.80-3.10 (m, 4H), 3.15-3.40 (m, 2H), 3.8-4.1 (m, 4H), 6.0-6.9 (bs, 3H), 7.0-7.3 (m, 3H).

### Preparation 8

### 5-(2-CHLORO-ETHYL)-INDAN-2-YL-AMMONIUM(D)(−)TARTRATE SALT

(±)2-indanamine (1 eq) and D(−)-tartaric acid (1 eq) was dissolved in a refluxing mixture of 50 mL ethanol and 10 mL water. After 6 hrs stirring, while the mixture was allowed to cool to rt, the obtained precipitate was collected, rinsed with ethanol/water(5/1) and dried. The crystalline salt was recrystallized twice. First from 50 mL ethanol/15 mL water and second with 50 mL ethanol and 20 mL water. 5-(2-chloro-ethyl)-indan-2-yl-ammonium (D)(−)Tartrate salt was isolated with an e.e. of 95% for the amine. ¹H NMR (200 MHz, D6-DMSO) δ 2.80-3.10 (m, 4H), 3.15-3.40 (m, 2H), 3.80-4.10 (m, 3H), 6.0-6.9 (bs, 3H), 7.1-7.3 (m, 3H).

### Preparation 9

### (R)—N-[5-(2-CHLORO-ETHYL)-INDAN-2-YL1-ACETAMIDE

To a slurry of 5-(2-chloro-ethyl)-indan-2-yl-ammonium (L)(+)Tartrate salt (4.00 g, 11.58 mmole) in Et₂O (150 ml) was added 2.5N NaOH (100 ml). The slurry was sonicated then diluted with Et₂O (100 ml) and the layers separated. The organics were washed with 2.5N NaOH (2×100 ml) and the aqueous layers back-extracted with Et₂O (100 ml). The combined organics were washed with water (100 ml), brine (50 ml), dried (MgSO₄) and filtered. The ethereal solution was treated with Et₃N (3.23 ml, 23.15 mmole) followed by dropwise addition of acetyl chloride (0.91 ml, 12.74 mmole). After stirring for 1 h, the reaction was quenched with water and the layers separated. The organics were washed with 2H HCl (2×50 ml), water (50 ml), brine (50 ml), dried (MgSO₄) and concentrated to a solid as (R)—N-[5-(2-Chloro-ethyl)-indan-2-yl]-acetamide (2.71 g, 11.58 mmole). 1H NMR (400 MHz, CHLOROFORM-D) δ ppm 1.92 (s, 3 H) 2.75 (dt, J16.30, 4.55 Hz, 2 H) 3.02 (t, J=7.33 Hz, 2 H) 3.23-3.30 (m, 2 H) 3.68 (t, J=7.45 Hz, 2 H) 4.67-4.75 (m, 1 H) 5.74 (s, 1 H) 7.02 (d, J=7.57 Hz, 1 H) 7.07 (s, 1 H) 7.16 (d, J=7.82 Hz, 1 H).

### Preparation 10

### (S)—N-[5-(2-CHLORO-ETHYL)-INDAN-2-YL]-ACETAMIDE

Starting with 5-(2-chloro-ethyl)-indan-2-yl-ammonium (D)(−)Tartrate salt (4.00 g, 11.58 mmole) and acetyl chloride (1.5 eq), and following the procedure as outlined in Preparation 9, (2.29 g, 9.65 mmole) of (S)—N-[5-(2-Chloro-ethyl)-indan-2-yl]-acetamide was prepared. 1H NMR (400 MHz, CHLOROFORM-D) δ ppm 1.93 (s, 3 H) 2.75 (dt, J=16.18, 4.61 Hz, 2 H) 3.02 (t, J=7.33 Hz, 2 H) 3.27 (dt, J=16.18, 5.95 Hz, 2 H) 3.69 (t, J=7.33 Hz, 2 H) 4.67-4.75 (m, 1 H) 5.69 (s, 1 H) 7.03 (d, J=7.57 Hz, 1 H) 7.08 (s, 1 H) 7.16 (d, J=7.81 Hz, 1 H).

### Preparation 11

### (R)—N-[5-(2-CHLORO-ETHYL)-INDAN-2-YL]-PROPIONAMIDE

Starting with 5-(2-chloro-ethyl)-indan-2-yl-ammonium (L)(+)Tartrate salt (2.00 g, 5.79 mmole) and proprionyl chloride (1.5 eq) and following the procedure as outlined in Preparation 9, (1.46 g, 5.79 mmole) of (R)—N-[5-(2-Chloro-ethyl)-indan-2-yl]-propionamide was isolated. 1H NMR (400 MHz, CHLOROFORM-D) δ ppm 1.07 (t, J=7.57 Hz, 3 H) 2.09 (q, J=7.65 Hz, 2 H) 2.69 (dt, J=16.30, 4.55 Hz, 2 H) 2.97 (t, J=7.45 Hz, 2 H) 3.23 (dt, J=16.12, 5.98 Hz, 2 H) 3.64 (t, J7.45 Hz, 2 H) 4.64-4.72 (m, 1 H) 5.58 (s, 1 H) 6.97 (d, J=7.57 Hz, 1 H) 7.03 (s, 1 H) 7.11 (d, J=7.57 Hz, 1 H).

### Preparation 12

### (S)—N-[5-(2-CHLORO-ETHYL)-INDAN-2-YL]-PROPIONAMIDE

Starting with 5-(2-chloro-ethyl)-indan-2-yl-ammonium (D)(−)Tartrate salt (2.50 g, 7.24 mmole) and proprionyl chloride (1.5 eq) and following the procedure as outlined in Preparation 9, (1.64 g, 6.52 mmole) of (S)—N-[5-(2-Chloro-ethyl)-indan-2-yl]-propionamide was isolated. 1H NMR (400 MHz, CHLOROFORM-D) δ ppm 1.07 (t, J=7.57 Hz, 3 H) 2.09 (q, J=7.57 Hz, 2 H) 2.69 (dt, J16.12, 4.64 Hz, 2 H) 2.97 (t, J=7.33 Hz, 2 H) 3.23 (dt, J=16.18, 5.95 Hz, 2 H) 3.64 (t, J=7.33 Hz, 2 H) 4.64-4.72 (m, 1 H) 5.58 (s, 1 H) 6.97 (d, J=7.57 Hz, 1 H) 7.03 (s, 1 H) 7.11 (d, J=7.81 Hz, 1 H).

### Preparation 13

### (R)-CYCLOPROPANECARBOXYLIC ACID[5-(2-CHLORO-ETHYL)-INDAN-2-YL]-AMIDE

Starting with 5-(2-chloro-ethyl)-indan-2-yl-ammonium (L)(+)Tartrate salt (3.10 g, 8.97 mmole) and Cyclopropanecarbonyl chloride (1.5 eq) and following the procedure as outlined in Preparation 9, (2.36 g, 8.95 mmole) of (R)-Cyclopropanecarboxylic acid[5-(2-chloro-ethyl)-indan-2-yl]-amide was isolated. 1H NMR (400 MHz, CHLOROFORM-D) δ ppm 0.61-0.67 (m, 2 H) 0.88-0.92 (m, 2 H) 1.15-1.23 (m, 1 H) 2.72 (dt, J=16.18, 4.61 Hz, 2 H) 2.98 (t, J=7.45 Hz, 2 H) 3.22 (ddd, J=16.18, 6.90, 4.52 Hz, 2 H) 3.64 (t, J=7.45 Hz, 2 H) 4.64-4.72 (m, 1 H) 5.74 (s, 1 H) 6.98 (d, J=7.82 Hz, 1 H) 7.04 (s, 1 H) 7.12 (d, J=7.57 Hz, 1 H).

### Preparation 14

### (S)-CYCLOPROPANECARBOXYLIC ACID[5-(2-CHLORO-ETHYL)-INDAN-2-YL]-AMIDE

Starting with 5-(2-chloro-ethyl)-indan-2-yl-ammonium (D)(−)Tartrate salt (3.08 g, 8.91 mmole) and Cyclopropanecarbonyl chloride (1.5 eq) and following the procedure as outlined in Preparation 9, (2.09 g, 7.94 mmole) of (S)-Cyclopropanecarboxylic acid[5-(2-chloro-ethyl)-indan-2-yl]-amide was isolated. 1H NMR (400 MHz, CHLOROFORM-D) δ ppm 0.67-0.72 (m, 2 H) 0.96 (ddd, J=7.08, 4.15, 3.91 Hz, 2 H) 1.19-1.28 (m, 1 H) 2.78 (ddd, J=15.94, 4.88, 4.58 Hz, 2 H) 3.03 (t, J=7.33 Hz, 2 H) 3.28 (ddd, J=16.18, 7.02, 4.88 Hz, 2 H) 3.69 (t, J=7.33 Hz, 2 H) 4.64-4.72 (m, 1 H) 5.80 (s, 1 H) 7.03 (d, J=8.06 Hz, 1 H) 7.09 (s, 1 H) 7.17 (d, J=7.81 Hz, 1 H).

### Preparation 15

### (R)—N-[5-(2-CHLORO-ETHYL)-INDAN-2-YL]-BUTYRAMIDE

Starting with 5-(2-chloro-ethyl)-indan-2-yl-ammonium (L)(+)Tartrate salt (3.05 g, 8.83 mmole) and Butyryl chloride (1.5 eq) and following the procedure as outlined in Preparation 9, (2.34 g, 8.80 mmole) of (R)—N-[5-(2-Chloro-ethyl)-indan-2-yl]-butyramide was isolated. 1H NMR (400 MHz, CHLOROFORM-D) δ ppm 0.91 (t, J=7.45 Hz, 3 H) 1.63 (hextet, J=7.39 Hz, 2 H) 2.06-2.11 (m, 2 H) 2.74 (dt, J=16.12, 4.64 Hz, 2 H) 3.03 (t, J=7.45 Hz, 2 H) 3.28 (ddd, J=16.12, 6.84, 5.13 Hz, 2 H) 3.69 (t, J=7.33 Hz, 2 H) 4.70-4.78 (m, 1 H) 5.61 (s, 1 H) 7.03 (d, J=7.57 Hz, 1 H) 7.08 (s, 1 H) 7.16 (d, J=7.57 Hz, 1 H).

### Preparation 16

### (S)—N-[5-(2-CHLORO-ETHYL)-INDAN-2-YL]-BUTYRAMIDE

Starting with 5-(2-chloro-ethyl)-indan-2-yl-ammonium (D)(−)Tartrate salt (3.00 g, 8.83 mmole) and Butyryl chloride (1.5 eq) and following the procedure as outlined in Preparation 9, (1.92 g, 7.24 mmole) of (S)—N-[5-(2-Chloro-ethyl)-indan-2-yl]-butyramide was isolated. 1H NMR (400 MHz, CHLOROFORM-D) δ ppm 0.91 (t, J=7.33 Hz, 3 H) 1.57-1.73 (m, 2 H) 2.06-2.11 (m, 2 H) 2.74 (dt, J=16.30, 4.55 Hz, 2 H) 3.03 (t, J=7.45 Hz, 2 H) 3.28 (ddd, J=16.30, 6.90, 4.88 Hz, 2 H) 3.69 (t, J=7.45 Hz, 2 H) 4.70-4.78 (m, 1 H) 5.62 (s, 1 H) 7.03 (d, J=7.57 Hz, 1 H) 7.08 (s, 1 H) 7.16 (d, J=7.81 Hz, 1 H).

### Preparation 17

### (R)—N-[5-(2-CHLORO-ETHYL)-INDAN-2-YL]-ISOBUTYRAMIDE

Starting with 5-(2-chloro-ethyl)-indan-2-yl-ammonium (L)(+)Tartrate salt (3.00 g, 8.68 mmole) and Isobutyryl chloride (1.5 eq) and following the procedure as outlined in Preparation 9, (2.29 g, 8.62 mmole) of (R)—N-[5-(2-Chloro-ethyl)-indan-2-yl]-isobutyramide was isolated. 1H NMR (400 MHz, CHLOROFORM-D) δ ppm 1.12 (d, J=6.84 Hz, 6 H) 2.25 (heptet, J=6.88 Hz, 1 H) 2.73 (dt, J=16.30, 4.55 Hz, 2 H) 3.03 (t, J=7.45 Hz, 2 H) 3.29 (ddd, J=16.24, 6.96, 4.88 Hz, 2 H) 3.69 (t, J=7.45 Hz, 2 H) 4.70-4.78 (m, 1 H) 5.61 (s, 1 H) 7.03 (d, J=7.57 Hz, 1 H) 7.08 (s, 1 H) 7.16 (d, J=7.82 Hz, 1 H).

### Preparation 18

### (S)—N-[5-(2-CHLORO-ETHYL)-INDAN-2-YL]-ISOBUTYRAMIDE

Starting with 5-(2-chloro-ethyl)-indan-2-yl-ammonium (D)(−)Tartrate salt (3.00 g, 8.68 mmole) and Isobutyryl chloride (1.5 eq) and following the procedure as outlined in Preparation 9, (1.99 g, 7.49 mmole) of (S)—N-[5-(2-Chloro-ethyl)-indan-2-yl]-isobutyramide was isolated. 1H NMR (400 MHz, CHLOROFORM-D) δ ppm 1.12 (d, J=6.84 Hz, 6 H) 2.25 (heptet, 6.88 Hz, 1 H) 2.73 (ddd, J=16.24, 4.64, 4.52 Hz, 2 H) 3.03 (t, J=7.33 Hz, 2 H) 3.29 (ddd, J=16.24, 6.96, 4.88 Hz, 2 H) 3.69 (t, J=7.33 Hz, 2 H) 4.69-4.78 (m, 1 H) 5.61 (s, 1 H) 7.03 (d, J=7.57 Hz, 1 H) 7.08 (s, 1 H) 7.16 (d, J=7.57 Hz, 1 H).

### Preparation 19

### (R)-ISOXAZOLE-5-CARBOXYLIC ACID[5-(2-CHLORO-ETHYL)-INDAN-2-YL]-AMIDE

Starting with 5-(2-chloro-ethyl)-indan-2-yl-ammonium (L)(+)Tartrate salt (3.00 g, 8.68 mmole) and Isobutyryl chloride (1.5 eq) and following the procedure as outlined in Preparation 9, (2.27, 7.81 mmole) of (R)-Isoxazole-5-carboxylic acid[5-(2-chloro-ethyl)-indan-2-yl]-amide was isolated. 1H NMR (400 MHz, CHLOROFORM-D) δ ppm 2.91 (ddd, J=16.24, 4.40, 4.27 Hz, 2 H) 3.04 (t, J=7.45 Hz, 2 H) 3.34-3.41 (m, 2 H) 3.70 (t, J=7.45 Hz, 2 H) 4.86-4.94 (m, 1 H) 6.76 (d, J=6.59 Hz, 1 H) 6.90 (d, J=1.71 Hz, 1 H) 7.06 (d, J=7.57 Hz, 1 H) 7.11 (s, 1 H) 7.20 (d, J=7.81 Hz, 1 H) 8.30 (d, J=1.95 Hz, 1 H).

### Preparation 20

### (S)-ISOXAZOLE-5-CARBOXYLIC ACID[5-(2-CHLORO-ETHYL)-INDAN-2-YL]-AMIDE

Starting with 5-(2-chloro-ethyl)-indan-2-yl-ammonium (D)(−)Tartrate salt (3.00 g, 8.68 mmole) and Isobutyryl chloride (1.5 eq) and following the procedure as outlined in Preparation 9, (2.01, 6.93 mmole) of (S)-Isoxazole-5-carboxylic acid[5-(2-chloro-ethyl)-indan-2-yl]-amide was isolated. 1H NMR (400 MHz, CHLOROFORM-D) δ ppm 2.91 (ddd, J=16.24, 4.40, 4.27 Hz, 2 H) 3.04 (t, J=7.33 Hz, 2 H) 3.38 (dt, J=16.12, 5.98 Hz, 2 H) 3.70 (t, J=7.45 Hz, 2 H) 4.86-4.94 (m, 1 H) 6.75 (s, 1 H) 6.90 (d, J=1.95 Hz, 1 H) 7.06 (d, J=7.57 Hz, 1 H) 7.11 (s, 1 H) 7.20 (d, J=7.57 Hz, 1 H) 8.30 (d, J=1.71 Hz, 1 H).

### Example 59

### (R)(−)-N-{5-[2-(4-BENZO[D]ISOTHIAZOL-3-YL-PIPERAZIN-1-YL)-ETHYL]-INDAN-2-YL}-ACETAMIDE

A slurry of (R)—N-[5-(2-Chloro-ethyl)-indan-2-yl]-acetamide (2.00 g, 8.41 mmole), Na₂CO₃ (1.5 eq) and 3-Piperazin-1-yl-benzo[d]isothiazole hydrochloride (2.0 eq) in H₂O (20 ml) was reacted under microwave assistance using a CEM MARS-5 microwave reactor to 175° C. for 10 min. Upon cooling, the reaction was diluted with EtOAc (250 ml), H₂O (100 ml) and the layers separated. The aqueous layer was extracted with EtOAc (2×50 ml). The organics were dried (MgSO₄), concentrated, and the residue purified by chromatography (EtOAc) to give (R)(−)-N-{5-[2-(4-Benzo[d]isothiazol-3-yl-piperazin-1-yl)-ethyl]-indan-2-yl}-acetamide (2.92 g, 6.94 mmole) in 100% purity @ 254 nm; LCMS (APCI) 474 [M+H]⁺. [α]_{D}²⁵−3.6° (c 5.5, CHCl₃). 1H NMR (400 MHz, CHLOROFORM-D) δ ppm 1.94 (s, 3 H) 2.65-2.71 (m, 2 H) 2.73-2.80 (m, 6 H) 2.81-2.87 (m, 2 H) 3.28 (ddd, J=16.04, 6.10, 5.92 Hz, 2 H) 3.57-3.62 (m, 4 H) 4.69-4.76 (m, 1 H) 5.70 (d, J=7.08 Hz, 1 H) 7.06 (d, J=7.81 Hz, 1 H) 7.11 (s, 1 H) 7.16 (d, J7.56 Hz, 1 H) 7.35 (ddd, J=8.17, 7.08, 1.10 Hz, 1 H) 7.46 (ddd, J=8.11, 7.02, 1.22 Hz, 1 H) 7.81 (dt, J=8.05, 0.85 Hz, 1 H) 7.91 (dt, J=8.30, 0.98 Hz, 1 H).

Using the appropriate chloroethylindanes from Preparations 9-20, Examples 60-68 were reacted in parallel format according to the procedure outlined in Example 59.

| Example | | |
|---|---|---|
| Number | Compound Name | Data |
| 60 | (S)(+)-N-{5-[2-(4- | 100% purity @ 254nm; |
| | BENZO[D]ISOTHIAZOL-3-YL- | LCMS(APCI) 474[M+H]⁺.[α]_{D}²⁵ +4.2° (c |
| | PIPERAZIN-1-YL)-ETHYL]- | 5.5, CHCl₃). 1H NMR(400MHz, |
| | INDAN-2-YL}-ACETAMIDE | CHLOROFORM-D) δ ppm 1.93(s, |
| | | 3H) 2.61-2.70(m, 2H) 2.72-2.79(m, |
| | | 6H) 2.80-2.86(m, 2H) 3.22-3.325(m, |
| | | 2H) 3.55-3.62(m, 4H) |
| | | 4.67-4.77(m, 1H) 5.66(d, J=6.84Hz, |
| | | 1H) 7.05(d, J=7.57Hz, 1H) |
| | | 7.10(s, 1H) 7.15(d, J=7.82Hz, |
| | | 1H) 7.34(ddd, J=8.18, 7.08, 1.10Hz, |
| | | 1H) 7.45(ddd, J=8.18, 6.96, 0.98Hz, |
| | | 1H) 7.80(ddd, J=8.18, 0.98, |
| | | 0.85Hz, 1H) 7.90(ddd, J=8.18, |
| | | 0.98, 0.85Hz, 1H). |
| 61 | (R)(−)-N-{5-[2-(4- | 100% purity @ 254nm; |
| | BENZO[D]ISOTHIAZOL-3-YL- | LCMS(APCI) 435[M+H]⁺.[α]_{D}²⁵−2.00° (c |
| | PIPERAZIN-1-YL)-ETHYL]- | 5.8, CHCl₃). 1H NMR(400MHz, |
| | INDAN-2-YL}-PROPIONAMIDE | CHLOROFORM-D) δ ppm 1.13(t, |
| | | J=7.57Hz, 3H) 2.15(q, J=7.57Hz, |
| | | 2H) 2.62-2.70(m, 2H) 2.71-2.78(m, |
| | | 6H) 2.80-2.86(m, 2H) 3.24-3.32(m, |
| | | 2H) 3.57-3.61(m, 4H) |
| | | 4.70-4.78(m, 1H) 5.59(d, J=7.06Hz, |
| | | 1H), 7.05(d, J=7.33Hz, 1H) |
| | | 7.10(s, 1H) 7.15(d, J=7.57Hz, 1H) |
| | | 7.34(ddd, J=8.12, 7.02, 0.98Hz, |
| | | 1H) 7.46(ddd, J=8.18, 6.96, 0.98Hz, |
| | | 1H) 7.80(dt, J=8.12, 0.95Hz, |
| | | 1H) 7.90(dt, J=8.12, 0.95Hz, 1H). |
| 62 | (S)(+)-N-{5-[2-(4- | 100% purity @ 254nm; |
| | BENZO[D]ISOTHIAZOL-3-YL- | LCMS(APCI) 435[M+H]⁺.[α]_{D}²⁵+5.7° (c |
| | PIPERAZIN-1-YL)-ETHYL]- | 6.3, CHCl₃). 1H NMR(400MHz, |
| | INDAN-2-YL}-PROPIONAMIDE | CHLOROFORM-D) δ ppm 1.12(t, |
| | | J=7.57Hz, 3H) 2.14(q, J=7.57Hz, |
| | | 2H) 2.62-2.70(m, 2H) 2.71-2.78(m, |
| | | 6H) 2.80-2.86(m, 2H) 3.28(ddd, |
| | | J=16.06, 6.23, 6.04Hz, 2H) |
| | | 3.56-3.61(m, 4H) 4.70-4.77(m, |
| | | 1H) 5.64(d, J=7.33Hz, 1H) 7.05(d, |
| | | J=7.57Hz, 1H) 7.10(s, 1H) 7.15(d, |
| | | J=7.82Hz, 1H) 7.34(ddd, |
| | | J=8.12, 7.02, 0.98Hz, 1H) 7.45(ddd, |
| | | J=8.12, 7.02, 1.22Hz, 1H) |
| | | 7.80(ddd, J=8.18, 0.85, 0.73Hz, 1H) |
| | | 7.90(ddd, J=8.18, 0.98, 0.85Hz, |
| | | 1H). |
| 63 | (R)(−)- | 95% purity @ 254nm; |
| | CYCLOPROPANECARBOXYLIC | LCMS(APCI) 447[M+H]⁺.[α]_{D}²⁵ −2.80° (c |
| | ACID {5-[2-(4- | 7.0, CHCl₃). 1H NMR(400MHz, |
| | BENZO[D]ISOTHIAZOL-3-YL- | CHLOROFORM-D) δ ppm 0.66-0.73(m, |
| | PIPERAZIN-1-YL)-ETHYL]- | 2H) 0.96(ddd, J=7.08, |
| | INDAN-2-YL}-AMIDE | 4.15, 3.91Hz, 2H) 1.20-1.28(m, |
| | | 1H) 2.64-2.72(m, 2H) 2.73-2.87(m, |
| | | 8H) 3.27(ddd, J=16.06, 6.78, 5.01Hz, |
| | | 2H) 3.54-3.63(m, 4H) 4.71-4.79(m, |
| | | 1H) 5.83(d, J=8.44Hz, |
| | | 1H) 7.05(d, J=9.04Hz, 1H) 7.11(s, |
| | | 1H) 7.16(d, J=7.82Hz, 1H) |
| | | 7.34(ddd, J=8.12, 7.02, 0.98Hz, 1H) |
| | | 7.46(ddd, J=8.12, 7.02, 0.98Hz, |
| | | 1H) 7.80(d, J=8.06Hz, 1H) |
| | | 7.90(d, J=8.06Hz, 1H). |
| 64 | (S)(+)- | 100% purity @ 254nm; |
| | CYCLOPROPANECARBOXYLIC | LCMS(APCI) 447[M+H]⁺. 1H NMR(400MHz, |
| | ACID {5-[2-(4- | CHLOROFORM-D) δ ppm |
| | BENZO[D]ISOTHIAZOL-3-YL- | 0.66-0.72(m, 2H) 0.95(ddd, |
| | PIPERAZIN-1-YL)-ETHYL]- | J=7.02, 4.21, 3.91Hz, 2H) 1.21-1.28(m, |
| | INDAN-2-YL}-AMIDE | 1H) 2.77(ddd, J=16.24, |
| | | 3.79, 3.66Hz, 2H) 2.84-2.96(m, |
| | | 4H) 2.97-3.02(m, 4H) 3.26(ddd, |
| | | J=16.24, 6.96, 3.42Hz, 2H) 3.70-3.75 |
| | | 3.75(m, 4H) 4.70-4.78(m, 1H) |
| | | 5.89(d, J=7.33Hz, 1H) 7.03(d, |
| | | J=7.57Hz, 1H) 7.09(s, 1H) 7.16(d, |
| | | J=7.57Hz, 1H) 7.36(ddd, |
| | | J=8.12, 7.02, 0.98Hz, 1H) 7.47(ddd, |
| | | J=8.12, 7.02, 1.22Hz, 1H) |
| | | 7.81(d, J=8.06Hz, 1H) 7.86(d, |
| | | J=8.06Hz, 1H). |
| 65 | (R)(−)-N-{5-[2-(4- | 100% purity @ 254nm; |
| | BENZO[D]ISOTHIAZOL-3-YL- | LCMS(APCI) 449[M+H]⁺.[α]_{D}²⁵ −3.30° (c |
| | PIPERAZIN-1-YL)-ETHYL]- | 7.1, CHCl₃). 1H NMR(400MHz, |
| | INDAN-2-YL}-BUTYRAMIDE | CHLOROFORM-D) δ ppm 0.91(t, |
| | | J=7.33Hz, 3H) 1.63(hextet, |
| | | J=7.42Hz, 2H) 2.08(t, J=7.42Hz, |
| | | 2H) 2.62-2.73(m, 3H) 2.74-2.78(m, |
| | | 5H) 2.80-2.86(m, 2H) 3.24-3.32(m, |
| | | 2H) 3.56-3.61(m, 4H) |
| | | 4.71-4.78(m, 1H) 5.63(d, J=8.45Hz, |
| | | 1H) 7.05(d, J=7.57Hz, 1H) |
| | | 7.09(s, 1H) 7.15(d, J=7.57Hz, |
| | | 1H) 7.34(ddd, J=8.12, 7.02, 0.98Hz, |
| | | 1H) 7.80(d, J=8.30Hz, 1H) |
| | | 7.90(d, J=8.30Hz, 1H). |
| 66 | (S)(+)-N-{5-[2-(4- | 100% purity @ 254nm; |
| | BENZO[D]ISOTHIAZOL-3-YL- | LCMS(APCI) 449[M+H]⁺.[α]_{D}²⁵ +7.3° (c |
| | PIPERAZIN-1-YL)-ETHYL]- | 6.0, CHCl₃). 1H NMR(400MHz, |
| | INDAN-2-YL}-BUTYRAMIDE | CHLOROFORM-D) δ ppm 0.91(t, |
| | | J=7.33Hz, 3H) 1.64(hextet, |
| | | J=7.42Hz, 2H) 2.09(t, J=7.42Hz, |
| | | 2H) 2.65-2.74(m, 3H) 2.74-2.78(m, |
| | | 5H) 2.79-2.86(m, 2H) 3.28(ddd, |
| | | J=16.30, 6.65, 5.37Hz, 2H) |
| | | 3.56-3.62(m, 4H) 4.71-4.78(m, |
| | | 1H) 5.62(d, J=7.63Hz, 1H) |
| | | 7.05d(J=7.57Hz, 1H) 7.10(s, 1H) |
| | | 7.15(d, J=7.57Hz, 1H) 7.34(ddd, |
| | | J=8.18, 6.96, 0.98Hz, 1H) 7.46(ddd, |
| | | J=8.06, 6.96, 1.10Hz, 1H) |
| | | 7.80(d, J=8.06Hz, 1H) 7.90(d, |
| | | J=8.06Hz, 1H). |
| 67 | (R)(−)-N-{5-[2-(4- | 95% purity @ 254nm; |
| | BENZO[D]ISOTHIAZOL-3-YL- | LCMS(APCI) 449[M+H]⁺.[α]_{D}²⁵ −3.6° (c |
| | PIPERAZIN-1-YL)-ETHYL]- | 7.7, CHCl₃). 1H NMR(400MHz, |
| | INDAN-2-YL}- | CHLOROFORM-D) δ ppm 1.12(d, |
| | ISOBUTYRAMIDE | J=6.84Hz, 6H) 2.25(heptet, J=6.88Hz, |
| | | 1H) 2.62-2.73(m, 3H) |
| | | 2.73-2.79(m, J=4.40Hz, 5H) 2.80-2.86(m, |
| | | 2H) 3.29(ddd, J=16.00, |
| | | 6.23, 6.11Hz, 2H) 3.56-3.62(m, |
| | | 4H) 4.69-4.77(m, 1H) 5.63(d, |
| | | J=7.33Hz, 1H) 7.05(d, J=7.82Hz, |
| | | 1H) 7.09(s, 1H) 7.15(d, J=7.57Hz, |
| | | 1H) 7.34(ddd, J=8.12, 7.02, |
| | | 0.98Hz, 1H) 7.45(ddd, J=8.12, |
| | | 7.02, 1.22Hz, 1H) 7.80(d, J=8.30Hz, |
| | | 1H) 7.90(d, J=8.30Hz, 1H). |
| 68 | (S)(+)-N-{5-[2-(4- | 100% purity @ 254nm; |
| | BENZO[D]ISOTHIAZOL-3-YL- | LCMS(APCI) 449[M+H]⁺.[α]_{D}²⁵ +5.2° (c |
| | PIPERAZIN-1-YL)-ETHYL]- | 5.4, CHCl₃). 1H NMR(400MHz, |
| | INDAN-2-YL}- | CHLOROFORM-D) δ ppm 1.12(d, |
| | ISOBUTYRAMIDE | J=6.84Hz, 6H) 2.25(heptet, J=6.84Hz, |
| | | 1H) 2.65-2.73(m, 3H) |
| | | 2.73-2.78(m, 5H) 2.80-2.86(m, |
| | | 2H) 3.25-3.33(m, 2H) 3.56-3.62(m, |
| | | 4H) 4.69-4.77(m, 1H) 5.63(d, |
| | | J=7.57Hz, 1H) 7.05(d, J=7.57Hz, |
| | | 1H) 7.09(s, 1H) 7.15(d, J=7.57Hz, |
| | | 1H) 7.34(ddd, J=8.12, 7.02, |
| | | 0.98Hz, 1H) 7.46(ddd, J=8.12, |
| | | 7.02, 1.22Hz, 1H) 7.80(d, J=8.06Hz, |
| | | 1H) 7.90(d, J=8.06Hz, 1H). |

### Example 69

### (R)(−)-N-{5-[2-(4-BENZO[D]ISOTHIAZOL-3-YL-PIPERAZIN-1-YL)-ETHYL]-INDAN-2-YL}-N-METHYL-ACETAMIDE HYDROCHLORIDE SALT

To a solution of (R)(−)-N-{5-[2-(4-Benzo[d]isothiazol-3-yl-piperazin-1-yl)-ethyl]-indan-2-yl}-acetamide (0.504 g, 1.199 mmole) in dry THF (10 ml) was added potassium tert-butoxide (0.161 g, 1.439 mmole) at rt and the reaction stirred for 10 min. To this stirring solution was added dropwise iodomethane (0.09 ml, 1.439 mmole) and the reaction stirred for 1 h. The reaction was heated to reflux for 1 h followed by cooling and quenching with water. The reaction was diluted with EtOAc and the layers separated. The aqueous layer was washed with 4N HCl (3×). The acidic aqueous was basified with KOH and extracted with DCM (3×), dried (MgSO₄), concentrated and the residue purified by chromatography (5% MeOH/DCM). The free-base was taken up in 1,4-dioxane and the HCL salt was precipitated upon treatment with 1N HCl Et₂O solution to give (R)(−)-N-{5-[2-(4-Benzo[d]isothiazol-3-yl-piperazin-1-yl)-ethyl]-indan-2-yl}-N-methyl-acetamide as its hydrochloride salt. 100% purity @ 254 nm; LCMS (APCI) 435 [M+H]⁺. [α]_{D}²⁵ −0.59° (c 6.7, CHCl₃). 1H NMR (400 MHz, CHLOROFORM-D) δ ppm 2.09 (s, CH3 rotomer, 2 H) 2.18 (s, CH3 rotomer, 1 H) 2.74-2.88 (m, 5 H) 2.94-3.05 (m, 1 H) 3.07-3.21 (m, 7H) 3.22-3.32 (m, 3 H) 3.47-3.61 (m, 2 H) 4.07-4.22 (m, 5 H) 4.68-4.78 (m, CH rotomer, 0.4H) 5.54-5.64 (m, CH rotomer, 0.6 H) 7.01-7.08 (m, 1 H) 7.09-7.18 (m, 2 H) 7.39 (t, J=7.45 Hz, 1 H) 7.51 (t, J=7.45 Hz, 1 H) 7.83 (t, J=9.16 Hz, 2 H) 13.31 (s, 1 H). Anal. Calc'd for C₂₅H₃₀N₄O1S1.1.0 HCl: C, 63.74; H, 6.63; N, 11.89. Found: C, 63.76; H, 6.55; N, 11.76.

### Example 70

### (S)(+)-N-{5-[2-(4-BENZO[D]ISOTHIAZOL-3-YL-PIPERAZIN-1-YL)-ETHYL]-INDAN-2-YL}-N-METHYL-ACETAMIDE HYDROCHLORIDE SALT

Starting with (S)(+)-N-{5-[2-(4-Benzo[d]isothiazol-3-yl-piperazin-1-yl)-ethyl]-indan-2-yl}-acetamide and iodomethane, and following the procedure as outlined in Example 69, (S)(+)-N-{5-[2-(4-Benzo[d]isothiazol-3-yl-piperazin-1-yl)-ethyl]-indan-2-yl}-N-methyl-acetamide was obtained as its Hydrochloride salt. 100% purity @ 254 nm; LCMS (APCI) 435 [M+H]⁺. [α]_{D}²⁵ +3.5° (c 7.9, CHCl₃). 1H NMR (400 MHz, CHLOROFORM-D) δ ppm 2.08 (s,CH3 rotomer, 2 H) 2.17 (s, CH3 rotomer 1 H) 2.75 (s, C(O)CH3 rotomer, 1 H) 2.79 (s, C(O)CH3 rotomer, 2 H) 2.83 (ddd, J=16.85, 6.47, 3.30 Hz, 1 H) 2.98 (dd, J=16.12, 6.11 Hz, 1 H) 3.07-3.21 (m, 6H) 3.22-3.30 (m, 2 H) 3.50-3.58 (m, 2 H) 4.07-4.19 (m, 4 H) 4.65-4.80 (m, CH rotomer, 0.35H) 5.48-5.63 (m, CH rotomer, 0.65H) 7.00-7.07 (m, 1 H) 7.09-7.16 (m, 2 H) 7.38 (t, J=7.57 Hz, 1 H) 7.50 (t, J=7.57 Hz, 1 H) 7.82 (t, J=8.79 Hz, 2 H) 13.24 (s, 1 H). Anal. Calc'd for C₂₅H₃₀N₄O1S1.1.07 HCl: C, 63.40; H, 6.61; N, 11.83. Found: C, 63.15; H, 6.60; N, 11.43.

### Example 71

### (R)—N-{5-[2-(4BENZO[D]ISOTHIAZOL-3-YL-PI PERAZIN-1-YL)-ETHYL]-INDAN-2-YL}-N-ETHYL-ACETAMIDE

Starting with (R)—N-{5-[2-(4-Benzo[d]isothiazol-3-yl-piperazin-1-yl)-ethyl]-indan-2-yl}-acetamide and iodoethane, and following the procedure as outlined in Example 69, (R)—N-{5-[2-(4-Benzo[d]isothiazol-3-yl-piperazin-1-yl)-ethyl]-indan-2-yl}-N-methyl-acetamide was obtained as its Hydrochloride salt. 100% purity @ 254 nm; LCMS (APCI) 449 [M+H]⁺. 1H NMR (400 MHz, CHLOROFORM-D) δ ppm 1.11-1.21 (m, 3 H) 1.97-2.05 (bs, 3 H) 2.12-2.22 (m, 3 H) 3.00 (ddd, J=15.78, 8.25, 3.71 Hz, 2 H) 3.06-3.22 (m, 6 H) 3.23-3.33 (m, 4 H) 3.54 (d, J=11.14 Hz, 2 H) 4.08-4.21 (m, 4 H) 4.62-4.71 (m, CH rotomer, 0.45H) 5.06-5.17 (m, CH rotomer, 0.55H) 6.99-7.16 (m, 3 H) 7.39 (t, J=7.52 Hz, 1 H) 7.51 (t, J=7.42 Hz, 1 H) 7.83 (t, J=8.60 Hz, 2 H) 13.26 (s, 1 H).

### Example 72

### (S)—N-{5-[2-(4BENZO[D]ISOTHIAZOL-3-YL-PIPERAZIN-1-YL)-ETHYL]-INDAN-2-YL}-N-ETHYL-ACETAMIDE

Starting with (S)—N-{5-[2-(4-Benzo[d]isothiazol-3-yl-piperazin-1-yl)-ethyl]-indan-2-yl}-acetamide and iodoethane, and following the procedure as outlined in Example 69, (S)—N-{5-[2-(4-Benzo[d]isothiazol-3-yl-piperazin-1-yl)-ethyl]-indan-2-yl}-N-methyl-acetamide was obtained as its Hydrochloride salt. 100% purity @ 254 nm; LCMS (APCI) 449 [M+H]⁺. 1H NMR (400 MHz, CHLOROFORM-D) δ ppm 1.02-1.16 (m, CH3 rotomers, 3 H) 2.08 (s, C(O)CH3 rotomer, 2 H) 2.11 (s, C(O)CH3 rotomer, 1 H) 2.88-3.00 (m, 2 H) 3.00-3.16 (m, 5 H) 3.18-3.28 (m, 4 H) 3.44-3.52 (m, 2 H) 3.64 (s, 3 H) 4.03-4.17 (m, 4 H) 5.54-5.69 (m, CH rotomer, 0.45H) 5.01-5.15 (m, CH rotomer, 0.55H) 6.95-7.12 (m, 3 H) 7.35 (t, J=7.62 Hz, 1 H) 7.45 (t, J=7.81 Hz, 1 H) 7.78 (t, J=8.47 Hz, 2 H) 13.24 (s, 1 H).

### Example 73

### (R)—N-{5-[2-(4-BENZO[D]ISOTHIAZOL-3-YL-PIPERAZIN-1-YL)-ETHYL]-INDAN-2-YL}-N-CYCLOPROPYLMETHYL-ACETAMIDE

Starting with (R)—N-{5-[2-(4-Benzo[d]isothiazol-3-yl-piperazin-1-yl)-ethyl]-indan-2-yl}-acetamide and Bromomethyl-cyclopropane, and following the procedure as outlined in Example 69, (R)—N-{5-[2-(4-Benzo[d]isothiazol-3-yl-piperazin-1-yl)-ethyl]-indan-2-yl}-N-cyclopropylmethyl-acetamide was obtained as its hydrochloride salt. 100% purity @ 254 nm; LCMS (APCI) 475 [M+H]⁺. 1H NMR (400 MHz, CHLOROFORM-D) δ ppm 0.01-0.05 (m, 2 H) 0.26-0.34 (m, 1 H) 0.38-0.46 (m, 1 H) 0.69-0.88 (m, 1 H) 2.00 (s, C(O)CH3 rotomer, 1.5 H) 2.04 (s, C(O)CH3 rotomer, 1.5 H) 2.90-3.06 (m, 10 H) 3.07-3.16 (m, 2 H) 3.32-3.42 (m, 2 H) 3.93-4.07 (m, 4 H) 4.49-4.59 (m, CH rotomer, 0.5H) 4.72-4.82 (m, CH rotomer, 0.5H) 6.83-7.02 (m, 3 H) 7.25 (t, J=7.57 Hz, 1 H) 7.36 (t, J=7.69 Hz, 1 H) 7.68 (dd, J=10.50, 8.30 Hz, 2 H) 13.15 (s, 1 H).

### Example 74

### (S)—N-{5-[2-(4-BENZO[D]ISOTHIAZOL-3-YL-PIPERAZIN-1-YL)-ETHYL]-INDAN-2-YL}-N-CYCLOPROPYLMETHYL-ACETAMIDE

Starting with (S)—N-{5-[2-(4-Benzo[d]isothiazol-3-yl-piperazin-1-yl)-ethyl]-indan-2-yl}-acetamide and Bromomethyl-cyclopropane, and following the procedure as outlined in Example 69, (S)—N-{5-[2-(4-Benzo[d]isothiazol-3-yl-piperazin-1-yl)-ethyl]-indan-2-yl}-N-cyclopropylmethyl-acetamide was obtained as its hydrochloride salt. 100% purity @ 254 nm; LCMS (APCI) 475 [M+H]⁺. 1H NMR (400 MHz, CHLOROFORM-D) δ ppm 0.01-0.05 (m, 2 H) 0.26-0.34 (m, 1 H) 0.39-0.46 (m, 1 H) 0.69-0.88 (m, 1 H) 2.00 (s, C(O)CH3 rotomer, 2H) 2.04 (s, C(O)CH3 rotomer, 1H) 2.90-3.08 (m, 10 H) 3.08-3.16 (m, 2 H) 3.40 (d, J=11.33 Hz, 2 H) 3.92-4.05 (m, 4 H) 4.48-4.59 (m, CH rotomer, 0.4H) 4.71-4.82 (m, CH rotomer, 0.6H) 6.84-7.02 (m, 3 H) 7.24 (t, J=7.52 Hz, 1 H) 7.36 (t, J=7.42 Hz, 1 H) 7.67 (t, J=8.79 Hz, 2 H) 13.05 (s, 1 H).

### Preparation 21

### 1,1,3,3-TETRAMETHYL-INDAN-2-ONE

Beilstein Registry Number 2048281; CAS Registry Number 5689-12-3 Star, J. E.; Eastman, R. H. J. Org. Chem. 1966, 31, 1393.

### Preparation 22

### 5-(2-CHLORO-ACETYL)-1,1,3,3-TETRAMETHYL-INDAN-2-ONE

Into a 250-mL round-bottom flask under N₂ was placed 1,1,3,3-tetramethylindanone (5.00 g, 26.5 mmol), aluminum chloride (21.2 g, 159 mmol), and methylene chloride (65 mL). Chloroacetyl chloride (3.37 mL, 42.4 mmol) was added dropwise and the reaction was heated to 40° C. for 5 h. After cooling down, the reaction mixture was poured into ice-water (100 mL) while stirring. The mixture was diluted with methylene chloride (250 mL). The organic layer was separated, washed with saturated NaHCO₃, water, brine, dried over Na₂SO₄, and evaporated. The residue was purified by chromatography (silica gel, 95:5 hexanes/ethyl acetate) to give 5-(2-Chloro-acetyl)-1,1,3,3-tetramethyl-indan-2-one (6.65 g, 95%) as a yellow solid: ¹H NMR (300 MHz, CDCl₃) δ 7.93-7.89 (m, 2H), 7.40 (dd, J=7.6, 0.9 Hz, 1H), 4.73 (s, 2H), 1.38 (s, 6H), 1.37 (s, 6H).

### Example 75

### 5-[2-(4-BENZO[D]ISOTHIAZOL-3-YL-PIPERAZIN-1-YL)-ACETYL]-1,1,3,3-TETRAMETHYL-INDAN-2-ONE

A mixture of 5-(2-Chloro-acetyl)-1,1,3,3-tetramethyl-indan-2-one (7.85 g, 29.6 mmol), 3-piperazin-1-yl-benzo[d]isothiazole hydrochloride (7.95 g, 31.1 mmol), potassium carbonate (13.5 g, 97.7 mmol), sodium iodide (4.50 g, 30.0 mmol) in acetonitrile (550 mL) was stirred at rt for 24 h. The reaction was quenched with water (120 mL), and acetonitrile was evaporated. The residue was extracted with methylene chloride (2×250 mL). The combined organic extracts were washed with water, brine, dried over Na₂SO₄, evaporated, and purified by chromatography (silica gel, 7:3 to 6:4 hexanes/ethyl acetate) to give 5-[2-(4-Benzo[d]isothiazol-3-yl-piperazin-1-yl)-acetyl]-1,1,3,3-tetramethyl-indan-2-one (11.2 g, 85%) as an off-white solid: ¹H NMR (300 MHz, CDCl₃) δ 8.02 (dd, J=8.0, 1.6 Hz, 1H), 7.96 (s, 1H), 7.91 (d, J=8.0 Hz, 1H), 7.83 (d, J=8.0 Hz, 1H), 7.47 (t, J=7.0 Hz, 1H), 7.38-7.34 (m, 2H), 3.94 (br s, 2H), 3.67-3.65 (m, 4H), 2.90-2.88 (m, 4H), 1.38 (s, 6H), 1.37 (s, 6H).

### Example 76

### 5-{2-[4-(1H-INDAZOL-3-YL)-PIPERAZIN-1-YL]-ACETYL}-1,1,3,3-TETRAMETHYL-INDAN-2-ONE

Starting with 5-(2-Chloro-acetyl)-1,1,3,3-tetramethyl-indan-2-one (3.00 g, 11.3 mmol), and 3-piperazin-1-yl-1H-indazole hydrochloride (2.76 g, 11.6 mmol), and following the procedure outlined in Example 75, a residue was isolated and purified by chromatography (silica gel, 3:2 to 4:1 EtOAc/hexanes with 0.5% of Et3N) to give 5-{2-[4-(1H-Indazol-3-yl)-piperazin-1-yl]-acetyl}-1,1,3,3-tetramethyl-indan-2-one (1.85 g, 37%) as a yellow solid: ¹H NMR (300 MHz, CDCl₃) δ 9.13 (s, 1H), 8.03 (dd, J=8.0, 1.6 Hz, 1H), 7.97 (d, J=1.4 Hz, 1 H), 7.72 (d, J=8.2 Hz, 1 H), 7.38-7.34 (m, 3H), 7.06 (m, 1H), 3.92 (s, 2H), 3.57 (m, 4H), 2.87 (m, 4H), 1.39 (s, 6H), 1.37 (s, 6H); ESI MS m/z 431 [C₂₆H₃₀N₄O₂+H]⁺.

### Example 77

### 5-[2-(4-BENZO[D]ISOTHIAZOL-3-YL-PIPERAZIN-1-YL)-1-HYDROXYETHYL]-1,1,3,3-TETRAMETHYL-INDAN-2-ONE

To a suspension of 5-[2-(4-Benzo[d]isothiazol-3-yl-piperazin-1-yl)-acetyl]-1,1,3,3-tetramethyl-indan-2-one (11.2 g, 25.0 mmol) in 2-propanol (650 mL) and methanol (650 mL) was added NaBH₄ (0.99 g, 26 mmol) at 0° C. After stirring at 0° C. for 8 h, the reaction was quenched with the addition of acetone (50 mL). The solvent was removed in vacuo. The residue was taken up in chloroform (1 L), washed with water, brine, dried over Na₂SO₄, evaporated, and purified by chromatography (silica gel, 7:3 to 6:4 hexanes/EtOAc) to give 5-[2-(4-Benzo[d]isothiazol-3-yl-piperazin-1-yl)-1-hydroxyethyl]-1,1,3,3-tetramethyl-indan-2-one (8.50 g, 76%) as a white solid: mp 189-191° C.; ¹H NMR (300 MHz, CDCl₃) δ 7.92 (d, J=8.1 Hz, 1H), 7.83 (d, J=8.1 Hz, 1H), 7.48 (td, J=7.0, 1.0 Hz, 1H), 7.40-7.30 (m, 3H), 7.24 (s, 1H), 4.83 (dd, J=10.0, 3.8 Hz, 1H), 4.03 (br s, 1H), 3.63-3.57 (m, 4H), 3.06-2.99 (m, 2H), 2.77-2.57 (m, 4H), 1.35 (s, 12H).

### Example 78

### 5-[2-(4-BENZO[D]ISOTHIAZOL-3-YL-PIPERAZIN-1-YL)-1-FLUORO-ETHYL]-1,1,3,3-TETRAMETHYL-INDAN-2-ONE

To a solution of 5-[2-(4-Benzo[d]isothiazol-3-yl-piperazin-1-yl)-1-hydroxyethyl]-1,1,3,3-tetramethyl-indan-2-one (1.50 g, 3.34 mmol) in CH₂Cl₂ (300 mL) was added DAST (0.53 mL, 4.0 mmol) slowly at 0° C. The mixture was stirred at 0° C. for 30 min, and then quenched with the addition of ice-water (20 mL). The organic layer was separated and washed with H₂O, brine, dried over Na₂SO₄, filtered, and the solvent was removed in vacuo. The residue was purified by flash column chromatography (silica gel, 7:3 hexanes/EtOAc) to give 5-[2-(4-Benzo[d]isothiazol-3-yl-piperazin-1-yl)-1-fluoro-ethyl]-1,1,3,3-tetramethyl-indan-2-one (1.00 g, 67%) as a pale-yellow solid: mp 64-73° C.; ¹H NMR (300 MHz, CDCl₃) δ 7.92 (d, J=8.1 Hz,1H), 7.83 (d, J=8.1 Hz, 1H), 7.48 (dd, J=7.8, 7.3 Hz, 1H), 7.36 (dd, J=7.8, 7.2 Hz, 1H), 7.33-7.28 (m, 3H), 5.75 (dd, J=48.8, 8.6 Hz, 1H), 3.64-3.61 (m, 4H), 3.12-2.70 (m, 6H), 1.36 (s, 6H), 1.35 (s. 6H); ESI MS m/z 452 [C₂₆H₃₀FN₃OS+H]⁺; R_{f} 0.27 (7:3 Hexanes/EtOAc); HPLC (Method B) 97.0% (AUC), t_{R}=15.17 min. Anal. Calc'd for C₂₆H₃₀FN₃OS.0.25H₂O: C, 68.47; H, 6.74; N, 9.21. Found: C, 68.17; H, 6.65; N, 8.97.

### Example 79

### 5-[2-(4-BENZO[D]ISOTHIAZOL-3-YL-PIPERAZIN-1-YL)-1-FLUORO-ETHYL]-1,1,3,3-TETRAMETHYL-INDAN-2-OL

To a suspension of 5-[2-(4-Benzo[d]isothiazol-3-yl-piperazin-1-yl)-1-fluoro-ethyl]-1,1,3,3-tetramethyl-indan-2-one (300 mg, 0.660 mmol) in 2-propanol (40 mL) and methanol (10 mL) was added NaBH₄ (62 mg, 1.6 mmol) at 0° C. The reaction was allowed to stir at rt for 24 h, and quenched with the addition of acetone (10 mL). After evaporating the solvent, the residue was taken up in chloroform (100 mL). The organic solution was washed with water, brine, dried over Na₂SO₄, evaporated. The residue was purified by chromatography (silica gel, 7:3 hexanes/ethyl acetate) to give 5-[2-(4-Benzo[d]isothiazol-3-yl-piperazin-1-yl)-1-fluoro-ethyl]-1,1,3,3-tetramethyl-indan-2-ol (290 mg, 97%) as a white solid: mp 49-54° C.; ¹H NMR (300 MHz, CDCl₃) δ 7.92 (d, J=8.1 Hz, 1H), 7.83 (d, J=8.1 Hz, 1H), 7.45 (td, J=7.2, 0.9 Hz, 1H), 7.36 (td, J=7.2, 0.9 Hz, 1H), 7.24-7.16 (m, 3H), 5.73 (dd, J=49.3, 8.9 Hz, 1H), 3.85 (d, J=8.2 Hz, 1H), 3.64-3.61 (m, 4H), 3.11-2.67 (m, 6H), 1.67 (d, J=8.2 Hz,1H), 1.38 (s, 3H), 1.37 (s, 3H), 1.20 (s, 3H), 1.19 (s, 3H); APCI MS m/z 454 [C₂₆H₃₂FN₃OS+H]⁺; R_{f} 0.29 (3:2 Hexanes/EtOAc); HPLC (Method B) >99% (AUC), t_{R}=14.24 min. Anal. Calc'd for C₂₆H₃₂FN₃OS.0.25H₂O: C, 68.17; H, 7.15; N, 9.17. Found: C, 68.03; H, 7.15; N, 8.86.

### Example80

### 5-[2-(4-BENZO[D]ISOTHIAZOL-3-YL-PIPERAZIN-1-YL)-1-HYDROXY-ETHYL]-1,1,3,3-TETRAMETHYL-INDAN-2-OL

To a suspension of 5-[2-(4-Benzo[d]isothiazol-3-yl-piperazin-1-yl)-acetyl]-1,1,3,3-tetramethyl-indan-2-one (500 mg, 1.12 mmol) in 2-propanol (70 mL) and methanol (20 mL) was added NaBH₄ (169 mg, 4.48 mmol) at 0° C. The reaction was allowed to stir at rt for 24 h, and quenched with the addition of acetone. After evaporating the solvent, the residue was taken up in chloroform (100 mL). The organic solution was washed with water, brine, dried over Na₂SO₄, evaporated. The residue was purified by chromatography (silica gel, 6:4 hexanes/EtOAc) to give the 5-[2-(4-Benzo[d]isothiazol-3-yl-piperazin-1-yl)-1-hydroxy-ethyl]-1,1,3,3-tetramethyl-indan-2-ol (450 mg, 89%) as a white solid: mp 88-97° C.; ¹H NMR (300 MHz, CDCl₃) δ 7.91 (d, J=8.1 Hz, 1H), 7.83 (d, J=8.1 Hz, 1H), 7.48 (td, J=7.0, 1.0 Hz, 1H), 7.37 (td, J=8.0, 1.0 Hz, 1H), 7.23-7.13 (m, 3H), 4.82 (dd, J=9.3, 4.3 Hz, 1H), 3.98 (br s, 1H), 3.83 (dd, J=8.3, 1.8 Hz, 1H), 3.66-3.56 (m, 4H), 3.05-2.97 (m, 2H), 2.76-2.68 (m, 2H), 2.66-2.56 (m, 2H), 1.38 (s, 3H), 1.36 (s, 3H), 1.19 (s, 3H), 1.18 (s, 3H); R_{f} 0.35 (3:2 EtOAc/hexanes); HPLC (Method B) >99% (AUC), t_{R}=13.51 min. Anal. Calc'd for C₂₆H₃₃N₃O₂S: C, 69.15; H, 7.36; N, 9.30. Found: C, 68.79; H, 7.46; N, 8.92.

### Example 81

### 5-[2-(4-BENZO[D]ISOTHIAZOL-3-YL-PIPERAZIN-1-YL)-1-CHLORO-ETHYL]-1,1,3,3-TETRAMETHYL-INDAN-2-ONE

To a solution of 5-[2-(4-Benzo[d]isothiazol-3-yl-piperazin-1-yl)-1-hydroxyethyl]-1,1,3,3-tetramethyl-indan-2-one (4.30 g, 9.56 mmol) in CH₂Cl₂ (300 mL) was added methanesulfonyl chloride (1.20 mL, 15.5 mmol), triethylamine (3.23 mL, 23.2 mmol) at 0° C. The reaction mixture was allowed to stir at rt for 2 h, and then quenched with water. The organic layer was separated, washed with water, brine, dried over Na₂SO₄, and evaporated. The residue was purified by chromatography (silica gel, 4:1 hexanes/EtOAc) to give 5-[2-(4-Benzo[d]isothiazol-3-yl-piperazin-1-yl)-1-chloro-ethyl]-1,1,3,3-tetramethyl-indan-2-one (3.80 g, 85%) as a white solid: ¹H NMR (300 MHz, CDCl₃) δ 7.90 (d, J=8.1 Hz,1H), 7.80 (d, J=8.1 Hz, 1H), 7.48 (dd, J=8.0, 0.9 Hz, 1H), 7.38-7.22 (m, 4H), 5.03 (dd, J=8.1, 5.8 Hz, 1H), 3.58-3.55 (m, 4H), 3.12 (dd, J=13.5, 8.1 Hz, 1H), 2.96 (dd, J=13.5, 8.1 Hz, 1H), 2.80-2.68 (m, 4H), 1.35 (s, 6H), 1.34 (s, 6H); ESI MS m/z 468 [C₂₆H₃₀ClN₃OS+H]⁺.

### Example 82

### 5-[2-(4-BENZO[D]ISOTHIAZOL-3-YL-PIPERAZIN-1-YL)-ETHYL]-1,1,3,3-TETRAMETHYL-INDAN-2-ONE

A solution of 5-[2-(4-Benzo[d]isothiazol-3-yl-piperazin-1-yl)-1-chloro-ethyl]-1,1,3,3-tetramethyl-indan-2-one (2.00 g, 4.27 mmol) in toluene (120 mL) in a sealed flask was degassed by bubbling argon for 5 min. The solution was then treated with tri-n-butyl tin hydride (1.73 mL, 6.40 mmol) and AIBN (105 mg, 0.640 mmol), and heated to 80° C. for 1 h. The reaction was cooled, and quenched with water. Toluene was removed in vacuo, and the residue was taken up in chloroform (500 mL). The organic layer was separated, washed with water, brine, dried over Na₂SO₄, and evaporated. The residue was purified by chromatography (silica gel, 7:3 hexanes/ethyl acetate) to provide 5-[2-(4-Benzo[d]isothiazol-3-yl-piperazin-1-yl)-ethyl]-1,1,3,3-tetramethyl-indan-2-one (1.65 g, 89%) as a white solid. The material was further purified by recrystallization from CH₂Cl₂/hexanes to provide white crystals: mp 122-126° C.; ¹H NMR (300 MHz, CDCl₃) [symbol] 7.93 (d, J=8.1 Hz, 1H), 7.82 (d, J=8.1 Hz, 1H), 7.47 (td, J=7.2, 0.9 Hz, 1H), 7.36 (td, J=7.2, 0.9 Hz, 1H), 7.19 (m, 2H), 7.13 (s, 1H), 3.63-3.60 (m, 4H), 2.94-2.70 (m, 8H), 1.34 (s, 6H), 1.33 (s, 6H); ESI MS m/z 434 [C₂₆H₃₁N₃OS+H]⁺; R_{f} 0.25 (3:2 hexanes/EtOAc); HPLC (Method B) >99% (AUC), t_{R}=15.04 min. Anal. Calc'd for C₂₆H₃₁N₃OS: C, 72.02; H, 7.21; N, 9.69. Found: C, 71.67; H, 7.29; N, 9.45.

### Example 83

### 5-[2-(4-BENZO[D]ISOTHIAZOL-3-YL-PIPERAZIN-1-YL)-ETHYL]-1,1,3,3-TETRAMETHYL-INDAN-2-OL

To a suspension of 5-[2-(4-Benzo[d]isothiazol-3-yl-piperazin-1-yl)-ethyl]-1,1,3,3-tetramethyl-indan-2-one (520 mg, 1.20 mmol) in 2-propanol (70 mL) and methanol (20 mL) was added NaBH₄ (113 mg, 3.00 mmol) at 0° C. The reaction was allowed to stir at rt for 36 h, and quenched with the addition of acetone. After evaporating the solvent, the residue was taken up in chloroform (150 mL). The organic solution was washed with water, brine, dried over Na₂SO₄, and purified by chromatography (silica gel, 3:2 hexanes/EtOAc) to give 5-[2-(4-Benzo[d]isothiazol-3-yl-piperazin-1-yl)-ethyl]-1,1,3,3-tetramethyl-indan-2-ol (420 mg, 81%) as a white solid: mp 47-49° C.; ¹H NMR (300 MHz, CDCl₃) δ 7.92 (d, J=8.2 Hz, 1 H), 7.82 (d, J=8.2 Hz, 1H), 7.47 (td, J=7.1, 0.8 Hz, 1H), 7.36 (td, J=7.1, 0.8 Hz, 1H), 7.12-7.08 (m, 2H), 7.02 (s, 1H), 3.84 (d, J=8.3 Hz, 1H), 3.63-3.60 (m, 4H), 2.89-2.67 (m, 8H), 1.65 (d, J=8.3 Hz, 1H), 1.37 (s, 3H), 1.36 (s, 3H), 1.19 (s, 3H), 1.18 (s, 3H); APCI MS m/z 436 [C₂₆H₃₃N₃OS+H]⁺; R_{f} 0.22 (3:2 hexanes/EtOAc); HPLC (Method B) 98.0% (AUC), t_{R}=14.16 min. Anal. Calc'd for C₂₆H₃₃N₃OS.0.25H₂O: C, 70.95; H, 7.67; N, 9.55. Found: C, 71.22; H, 7.74; N, 9.37.

### Example 84

### 5-[2-(4-BENZO[D]ISOTHIAZOL-3-YL-PIPERAZIN-1-YL)-1-METHYLAMINO-ETHYL]-1,1,3,3-TETRAMETHYL-INDAN-2-ONE

A mixture of 5-[2-(4-Benzo[d]isothiazol-3-yl-piperazin-1-yl)-1-chloro-ethyl]-1,1,3,3-tetramethyl-indan-2-one (500 mg, 1.07 mmol) and methylamine (2M in THF, 100 mL) in a sealed vessel was heated at 100° C. for 5 h. After cooling down the solvent was evaporated under reduced pressure. The residue was taken up in a mixture of ethyl acetate (150 mL) and saturated NaHCO₃ (20 mL). The organic layer was separated, washed with brine, dried over Na₂SO₄, and evaporated to give 5-[2-(4-Benzo[d]isothiazol-3-yl-piperazin-1-yl)-1-methylamino-ethyl]-1,1,3,3-tetramethyl-indan-2-one (500 mg, 100%) as a crude product: ¹H NMR (300 MHz, CDCl₃) δ 7.91 (d, J=8.1 Hz, 1H), 7.81 (d, J=8.1 Hz, 1H), 7.47 (dt, J=7.0, 1.0 Hz, 1H), 7.38 (dt, J=7.0, 1.0 Hz, 1H), 7.28-7.21 (m, 3H), 3.74 (dd, J=11, 3.3 Hz, 1H), 3.64-3.54 (m, 4H), 2.88-2.87 (m, 2H), 2.66-2.62 (m, 3H), 2.52-2.47 (m, 1H), 2.37 (s, 3H), 1.34 (s, 12H).

### Example 85

### 5-[2-(4-BENZO[D]ISOTHIAZOL-3-YL-PIPERAZIN-1-YL)-1-METHYLAMINO-ETHYL]-1,1,3,3-TETRAMETHYL-INDAN-2-OL

To a suspension of 5-[2-(4-Benzo[d]isothiazol-3-yl-piperazin-1-yl)-1-methylamino-ethyl]-1,1,3,3-tetramethyl-indan-2-one (500 mg, 1.08 mmol) in 2-propanol (40 mL) and methanol (40 mL) was added NaBH₄ (102 mg, 2.70 mmol) at 0° C. The reaction was allowed to stir at rt for 36 h, and quenched with the addition of acetone. After evaporating the solvent, the residue was taken up in ethyl acetate (150 mL). The organic solution was washed with water, brine, dried over Na₂SO₄, and purified by chromatography (silica gel, 100:5:0.5 CH₂Cl₂/MeOH/Et₃N) to give 5-[2-(4-Benzo[d]isothiazol-3-yl-piperazin-1-yl)-1-methylamino-ethyl]-1,1,3,3-tetramethyl-indan-2-ol_(300 mg) as a white solid: mp 96-101° C.; ¹H NMR (300 MHz, CDCl₃) δ 7.91 (d, J=8.1 Hz, 1H), 7.82 (d, J=8.1 Hz, 1H), 7.47 (td, J=7.1, 0.6 Hz, 1H), 7.36 (t, J=7.3 Hz, 1H), 7.21-7.17 (m, 2H), 7.12 (d, J=7.6 Hz, 1H), 3.85 (d, J=5.1 Hz, 1H), 3.69 (dd, J=10.9, 3.2 Hz, 4H), 3.59-3.53 (m, 4H), 2.90-2.84 (m, 2H), 2.66-2.42 (m, 5H), 2.34 (s, 1H), 1.69 (d, J=7.2 Hz, 1H), 1.38 (s, 3H), 1.36 (s, 3H), 1.19 (s, 6H); APCI MS m/z 465 [C₂₇H₃₆N₄OS+H]⁺; R_{f} 0.42 (9:1 chloroform/methanol); HPLC (Method B) 97.3% (AUC), t_{R}=13.44 min. Anal. Calc'd for C₂₇H₃₆N₄OS.0.375H₂O: C, 68.79; H, 7.86; N, 11.88. Found: C, 68.99; H, 7.85; N, 11.53.

### Example 86

### 5-{1-HYDROXY-2-[4-(1H-INDAZOL-3-YL)-PIPERAZIN-1-YL]-ETHYL}-1,1,3,3-TETRAMETHYL-INDAN-2-OL

To a suspension of 5-{2-[4-(1H-Indazol-3-yl)-piperazin-1-yl]-acetyl}-1,1,3,3-tetramethyl-indan-2-one (650 mg, 1.51 mmol) in 2-propanol (40 mL) and methanol (60 mL) was added NaBH₄ (228 mg, 6.04 mmol) at 0° C. The reaction was allowed to stir at rt for 24 h, and quenched with the addition of acetone. After evaporating the solvent, the residue was taken up in ethyl acetate (200 mL). The organic solution was washed with water, brine, dried over Na₂SO₄, evaporated. The residue was purified by chromatography (silica gel, 20:1 CH₂Cl₂/MeOH) to give the 5-{1-hydroxy-2-[4-(1H-Indazol-3-yl)-piperazin-1-yl]-ethyl}-1,1,3,3-tetramethyl-indan-2-ol (486 mg, 74%) as a pale yellow solid: mp 104-110° C.; ¹H NMR (300 MHz, CDCl₃) δ 9.26 (s, 1H), 7.72 (d, J=8.1 Hz, 1H), 7.35-7.31 (m, 2H), 7.24-7.04 (m, 4H), 4.80 (dd, J=9.0, 4.9 Hz, 1H), 4.07(s, 1H), 3.84 (d, J=7.0 Hz, 1H), 3.55-3.48 (m, 4H), 3.04-2.98 (m, 2H), 2.75-2.60 (m, 6H), 1.72 (d, J=8.2 Hz, 1H), 1.37 (s, 3H), 1.36 (s, 3H), 1.19 (s, 3H), 1.18 (s, 3H); ESI MS m/z 435 [C₂₆H₃₄N₄O₂+H]⁺; R_{f} 0.20 (20:1 CH₂Cl₂/MeOH); HPLC (Method B) 96.4% (AUC), t_{R}=12.50 min. Anal. Calc'd for C₂₆H₃₄N₄O₂.0.25H₂O: C, 71.12; H, 7.92; N, 12.76. Found: C, 71.18; H, 7.89; N, 12.57.

### Example 87

### 5-[2-(4-BENZO[D]ISOXAZOL-3-YL-PIPERAZIN-1-YL)-ACETYL]-1,1,3,3-TETRAMETHYL-INDAN-2-ONE

A mixture of 5-(2-Chloro-acetyl)-1,1,3,3-tetramethyl-indan-2-one (9.20 g, 34.7 mmol), 3-piperazin-1-yl-benzo[d]isoxazole hydrochloride (8.33 g, 34.7 mmol), potassium carbonate (15.8 g, 114 mmol), sodium iodide (5.17 g, 34.7 mmol) in acetonitrile (600 mL) was heated to reflux for 4 h. The solvent was evaporated in vacuo, and the residue was trituated with water (300 mL). The resultant precipitate was collected by filtration to give 5-[2-(4-Benzo[d]isoxazol-3-yl-piperazin-1-yl)-acetyl]-1,1,3,3-tetramethyl-indan-2-one (15.0 g, 100%) as an yellow solid: ¹H NMR (300 MHz, CDCl₃) δ 8.00 (dd, J=8.0, 1.6 Hz, 1H), 7.95 (d, J=1.3 Hz, 1H), 7.70 (d, J=8.0 Hz, 1H), 7.50-7.47 (m, 2H), 7.37 (d, J=7.9 Hz, 1H), 7.25-7.20 (m, 1H), 3.92 (s, 2H), 3.72-3.66 (m, 4H), 2.87-2.81 (m, 4H), 1.39 (s, 6H), 1.35 (s, 6H).

### Example 88

### 5-[2-(4-BENZO[D]ISOXAZOL-3-YL-PIPERAZIN-1-YL)-1-HYDROXY-ETHYL]-1,1,3,3-TETRAMETHYL-INDAN-2-ONE

A suspension of 5-[2-(4-Benzo[d]isoxazol-3-yl-piperazin-1-yl)-acetyl]-1,1,3,3-tetramethyl-indan-2-one (15.0 g, 34.7 mmol) in 2-propanol (800 mL) and methanol (800 mL) was warmed until the solution became clear. The solution was then cooled to 0° C., and treated with NaBH₄ (1.38 g, 36.5 mmol). After stirring at 0° C. for 4 h, the reaction was quenched with the addition of acetone. The solvent was removed in vacuo. The residue was taken up in methylene chloride (800 mL), washed with water, brine, dried over Na₂SO₄, evaporated, and purified by chromatography (silica gel, 7:3 to 6:4 hexanes/EtOAc) to give compound 5-[2-(4-Benzo[d]isoxazol-3-yl-piperazin-1-yl)-1-hydroxy-ethyl]-1,1,3,3-tetramethyl-indan-2-one (12.3 g, 82%) as a white solid: ¹H NMR (300 MHz, CDCl₃) δ 7.70 (d, J=8.0 Hz, 1H), 7.51-7.46 (m, 2H), 7.33-7.27 (m, 2H), 7.24-7.21 (m, 2H), 4.83 (dd, J=9.8, 4.0 Hz, 1H), 3.95 (s, 1H), 3.68-3.61 (m, 4H), 3.03-2.96 (m, 2H), 2.74-2.56 (m, 4H), 1.35 (s, 6H), 1.34 (s, 6H); ESI MS m/z 434 [C₂₆H₃₁N₃O₃+H]⁺.

### Example 89

### 5-[2-(4-BENZO[D]ISOXAZOL-3-YL-PIPERAZIN-1-YL)-1-CHLORO-ETHYL]-1,1,3,3-TETRAMETHYL-INDAN-2-ONE

To a solution of 5-[2-(4-Benzo[d]isoxazol-3-yl-piperazin-1-yl)-1-hydroxy-ethyl]-1,1,3,3-tetramethyl-indan-2-one (8.30 g, 19.2 mmol) in CH₂Cl₂ (400 mL) was added methanesulfonyl chloride (1.78 mL, 23.0 mmol), triethylamine (4.00 mL, 28.7 mmol) at 0° C. The reaction mixture was allowed to stir at rt for 1 h, and then quenched with water. The organic layer was separated, washed with water, brine, dried over Na₂SO₄, and evaporated. The residue was purified by chromatography (silica gel, 20:3 to 20:5 hexanes/ethyl acetate) to give 5-[2-(4-Benzo[d]isoxazol-3-yl-piperazin-1-yl)-1-chloro-ethyl]-1,1,3,3-tetramethyl-indan-2-one (5.50 g, 64%) as a pale yellow semi-solid: ¹H NMR (300 MHz, CDCl₃) δ 7.67 (d, J=8.1 Hz, 1H), 7.48-7.38 (m, 2H), 7.35 (d, J=1.7 Hz, 1H), 7.30-7.19 (m, 4H), 5.04 (dd, J=8.1, 5.8 Hz, 1H), 3.58-3.55 (m, 4H), 3.13 (dd, J=13.6, 8.2 Hz, 1H), 2.92 (dd, J=13.6, 8.2 Hz, 1H), 2.78-2.68 (m, 4H), 1.35 (s, 6H), 1.34 (s, 6H); ESI MS m/z 452 [C₂₆H₃₀ClN₃O₂+H]⁺.

### Example 90

### 5-[2-(4-BENZO[D]ISOXAZOL-3-YL-PIPERAZIN-1-YL)-ETHYL]-1,1,3,3-TETRAMETHYL-INDAN-2-ONE METHANESULFONATE

A solution of 5-[2-(4-Benzo[d]isoxazol-3-yl-piperazin-1-yl)-1-chloro-ethyl]-1,1,3,3-tetramethyl-indan-2-one (4.90 g, 10.8 mmol) in toluene (150 mL) in a sealed flask was degassed by bubbling N₂ for 5 min. The solution was then treated with tri-n-butyl tin hydride (4.38 mL, 16.2 mmol) and AIBN (0.268 g, 1.63 mmol), and heated to 80° C. for 1.5 h. The reaction was cooled, and quenched with water. Toluene was removed in vacuo, and the residue was taken up in methylene chloride (100 mL). The organic layer was separated, washed with water, brine, dried over Na₂SO₄, and evaporated. The residue was purified by chromatography (silica gel, 6:4 hexanes/EtOAc) to provide 5-[2-(4-Benzo[d]isoxazol-3-yl-piperazin-1-yl)-ethyl]-1,1,3,3-tetramethyl-indan-2-one (4.30 g, 95%) as a pale yellow solid. To a solution of 5-[2-(4-Benzo[d]isoxazol-3-yl-piperazin-1-yl)-ethyl]-1,1,3,3-tetramethyl-indan-2-one (530 mg, 1.27 mmol) in ethyl acetate (10 mL) was treated with CH₃SO₃H (2M in Et₂O, 0.64 mL, 1.28 mmol). The reaction mixture was stirred at rt for 15 min. The precipitate was collected by filtration and dried in a vacuum oven at 55° C. overnight to give 5-[2-(4-Benzo[d]isoxazol-3-yl-piperazin-1-yl)-ethyl]-1,1,3,3-tetramethyl-indan-2-one methanesulfonate (570 mg, 88%) as a white solid: mp 259-261° C.; ¹ H NMR (300 MHz, DMSO-d₆) [symbol] 9.94 (br s, 1H), 8.08 (d, J=8.1 Hz, 1H), 7.67-7.61 (m, 2H), 7.41-7.33 (m, 3H), 7.26 (dd, J=7.8, 1.3 Hz, 1H), 4.20 (d, J=12.7 Hz, 2H), 3.73 (d, J=11.2 Hz, 2H), 3.49-3.33 (m, 6H), 3.11-3.05 (m, 2H), 2.35 (s, 3H), 1.28 (s, 6H), 1.26 (s, 6H); ESI MS m/z 418 [C₂₆H₃₁N₃O2+H]⁺; R_{f} 0.22 (3:2 hexanes/EtOAc); HPLC >99% (AUC), t_{R}=14.76 min. Anal. Calc'd for C₂₆H₃₁N₃O₂.CH₃SO₃H: C, 63.13; H, 6.87; N, 8.18. Found: C, 62.98; H, 6.89; N, 8.11.

### Example 91

### 5-[2-(4-BENZO[D]ISOXAZOL-3-YL-PIPERAZIN-1-YL)-1-HYDROXYL-ETHYL]-1,1,3,3-TETRAMETHYL-INDAN-2-OL

To a suspension of 5-[2-(4-Benzo[d]isoxazol-3-yl-piperazin-1-yl)-acetyl]-1,1,3,3-tetramethyl-indan-2-one (2.1 g, 4.85 mmol) in 2-propanol (100 mL) and methanol (100 mL) was added NaBH₄ (0.370 g, 9.70 mmol). The reaction was allowed to stir at rt for 24 h, and quenched with the addition of acetone. After evaporating the solvent, the residue was taken up in methylene chloride (300 mL). The organic solution was washed with water, brine, dried over Na₂SO₄, and evaporated to give 5-[2-(4-Benzo[dz]isoxazol-3-yl-piperazin-1-yl)-1-hydroxyl-ethyl]-1,1,3,3-tetramethyl-indan-2-ol (2.00 g, 95%) as a pale yellow foam: ¹H NMR (300 MHz, CDCl₃) δ 7.70 (d, J=8.1 Hz, 1H), 7.52-7.45 (m, 2H), 7.35-7.12 (m, 4H), 4.80 (dd, J=8.5, 5.3 Hz, 1H), 3.89-3.82 (m, 2H), 3.66-3.59 (m, 4H), 3.01-2.94 (m, 2H), 2.73-2.59 (m, 4H), 1.72 (d, J=8.2 Hz, 1H), 1.37 (s, 3H), 1.35 (s, 3H), 1.19 (s, 3H), 1.17 (s, 3H); ESI MS m/z 436 [C₂₆H₃₃N₃O₃+H]⁺.

### Example 92

### 5-[2-(4-BENZO[D]ISOXAZOL-3-YL-PIPERAZIN-1-YL)-1-CHLORO-ETHYL]-1,1,3,3-TETRAMETHYL-INDAN -2-OL

To a solution of 5-[2-(4-Benzo[d]isoxazol-3-yl-piperazin-1-yl)-1-hydroxyl-ethyl]-1,1,3,3-tetramethyl-indan-2-ol (2.00 g, 4.59 mmol) in CH₂Cl₂ (80 mL) was added methanesulfonyl chloride (0.36 mL, 4.59 mmol) and triethylamine (0.80 mL, 5.74 mmol) at 0° C. The reaction mixture was allowed to stir at 0° C. for 2 h, and then quenched with water. The organic layer was separated, washed with water, brine, dried over Na₂SO₄, and evaporated. The residue was purified by chromatography (silica gel, 5:1 hexanes/EtOAc) to give 5-[2-(4-Benzo[d]isoxazol-3-yl-piperazin-1-yl)-1-chloro-ethyl]-1,1,3,3-tetramethyl-indan-2-ol (600 mg, 29%) as a pale yellow semi-solid: ¹H NMR (300 MHz, CDCl₃) δ 7.68 (d, J=8.1 Hz, 1H), 7.51-7.43 (m, 2H), 7.29-7.13 (m, 4H), 5.02 (dd, J=8.2, 5.6 Hz, 1H), 3.84 (d, J=8.2 Hz, 1H), 3.59-3.55 (m, 4H), 3.11 (dd, J=13.6, 8.3 Hz,1H), 2.89 (dd, J=13.6, 5.6 Hz,1H), 2.76-2.73 (m, 4H), 1.37 (s, 3H), 1.36 (s, 3H), 1.19 (s, 3H), 1.18 (s, 3H).

### Example 93

### 5-[2-(4-BENZO[D]ISOXAZOL-3-YL-PIPERAZIN-1-YL)-ETHYL]-1,1,3,3-TETRAMETHYL-INDAN-2-OL METHANESULFONATE

A solution of 5-[2-(4-Benzo[d]isoxazol-3-yl-piperazin-1-yl)-1-chloro-ethyl]-1,1,3,3-tetramethyl-indan-2-ol (580 mg, 1.28 mmol) in toluene (50 mL) in a sealed flask was degassed by bubbling N₂ for 10 min. The solution was then treated with tri-n-butyl tin hydride (0.52 mL, 1.92 mmol) and AIBN (31 mg, 0.19 mmol), and heated to 80° C. for 1.5 h. The reaction was cooled, and quenched with water. Toluene was removed in vacuo, and the residue was taken up in methylene chloride (100 mL). The organic layer was separated, washed with water, brine, dried over Na₂SO₄, and evaporated. The residue was purified by chromatography (silica gel, 6:4 hexanes/EtOAc) to give the free base of 5-[2-(4-Benzo[d]isoxazol-3-yl-piperazin-1-yl)-ethyl]-1,1,3,3-tetramethyl-indan-2-ol (380 mg) as a white foam. To a solution of 5-[2-(4-Benzo[d]isoxazol-3-yl-piperazin-1-yl)-ethyl]-1,1,3,3-tetramethyl-indan-2-ol (free base, 380 mg, 0.910 mmol) in ethyl acetate (10 mL) was treated with CH₃SO₃H (2M in Et₂O, 0.45 mL, 0.90 mmol). The reaction mixture was stirred at rt for 20 min. The precipitate was collected by filtration and dried in a vacuum oven at 55° C. overnight to give 5-[2-(4-Benzo[d]isoxazol-3-yl-piperazin-1-yl)-ethyl]-1,1,3,3-tetramethyl-indan-2-ol methanesulfonate (400 mg, 61%) as a white solid: mp 245-247° C.; ¹H NMR (300 MHz, DMSO-d₆) [symbol] 9.87 (br s, 1H), 8.08 (d, J=8.1 Hz, 1H), 7.65-7.60 (m, 2H), 7.38-7.33 (m, 1H), 7.15-7.09 (m, 3H), 5.08 (br s, 1H), 4.19 (d, J=12.4 Hz, 2H), 3.72 (d, J=11.1 Hz, 2H), 3.65 (s, 1H), 3.46-3.32 (m, 6H), 3.03-2.98 (m, 2H), 2.34 (s, 3H), 1.27 (s, 3H), 1.25 (s, 3H), 1.07 (s, 3H), 1.05 (s, 3H); ESI MS m/z 420 [C₂₆H₃₃N₃O₂+H]⁺; R_{f} 0.30 (3:2 hexanes/EtOAc); HPLC >99% (AUC), t_{R}=13.48 min. Anal. Calc'd for C₂₆H₃₃N₃O₂.CH₃SO₃H: C, 62.89; H, 7.23; N, 8.15. Found: C, 62.57; H, 7.08; N, 8.04.

### Example 94

### 5-[2-(4-BENZO[D]ISOXAZOL-3-YL-PIPERAZIN-1-YL)-1-ETHOXY-ETHYL]-1,1,3,3-TETRAMETHYL-INDAN-2-ONE METHANESULFONATE

To a solution of 5-[2-(4-Benzo[d]isoxazol-3-yl-piperazin-1-yl)-1-chloro-ethyl]-1,1,3,3-tetramethyl-indan-2-one (500 mg, 1.11 mmol) in ethanol (30 mL) was added palladium on carbon (50 mg, 10% Pd, 50% wet) and four drops of triethylamine. The mixture was then hydrogenated at 30 psi for overnight. The reaction mixture was filtered via a pad of Celite®. The filtrate was concentrated, and the residue was purified by chromatography (silica gel, 6:4 hexanes/EtOAc) to give 5-[2-(4-Benzo[d]isoxazol-3-yl-piperazin-1-yl)-1-ethoxy-ethyl]-1,1,3,3-tetramethyl-indan-2-one (free base, 400 mg) as a white solid. To a solution of 5-[2-(4-Benzo[d]isoxazol-3-yl-piperazin-1-yl)-1-ethoxy-ethyl]-1,1,3,3-tetramethyl-indan-2-one (free base, 400 mg, 0.87 mmol) in ethyl acetate (6 mL) was treated with CH₃SO₃H (2M in Et₂O, 0.43 mL, 0.87 mmol). The reaction mixture was stirred at rt for 10 min. The precipitate was collected by filtration and dried in a vacuum oven at 55° C. overnight to give 5-[2-(4-Benzo[d]isoxazol-3-yl-piperazin-1-yl)-1-ethoxy-ethyl]-1,1,3,3-tetramethyl-indan-2-one methanesulfonate (430 mg, 66%) as a white solid: mp 219-222° C.; ¹H NMR (300 MHz, CDCl₃) [symbol] 11.80 (br s, 1H), 7.64 (d, J=8.0 Hz, 1H), 7.59-7.50 (m, 2H), 7.37-7.25 (m, 4H), 5.24 (dd, J=9.8, 8.5 Hz, 1H), 4.21-3.91 (m, 5H), 3.66 (d, J=12.5 Hz, 1H), 3.54-3.42 (m, 2H), 3.35-3.12 (m, 4H), 2.90 (s, 3H), 1.34 (s, 6H), 1.33 (s, 6H); ESI MS m/z 462 [C₂₈H₃₅N₃O₃+H]⁺; R_{f} 0.32 (3:2 hexanes/EtOAc); HPLC 97.2% (AUC), t_{R}=15.72 min. Anal. Calc'd for C₂₈H₃₅N₃O₃.CH₃SO₃H: C, 62.45; H, 7.05; N, 7.53. Found: C, 62.29; H, 6.95; N, 7.17.

### Preparation 23

### 5-(3-CHLORO-PROPIONYL)-1,1,3,3-TETRAMETHYL-INDAN-2-ONE

Into a 250-mL round-bottom flask under N₂ was placed tetramethylindanone (7.00 g, 37.2 mmol), aluminum chloride (29.7 g, 223 mmol), and methylene chloride (90 mL). 3-chloropropionyl chloride (5.68 mL, 59.5 mmol) was added dropwise and the reaction was heated to 40° C. for 5 h. After cooling down, the reaction mixture was poured into ice-water (150 mL) while stirring. The mixture was diluted with methylene chloride (500 mL). The organic layer was separated, washed with saturated NaHCO₃, water, brine, dried over Na₂SO₄, and evaporated. The residue was purified by chromatography (silica gel, 93:7 to 90:10 hexanes/ethyl acetate) to give 5-(3-Chloro-propionyl)-1,1,3,3-tetramethyl-indan-2-one (5.00 g, 48%) as a yellow solid: ¹H NMR (300 MHz, CDCl₃) δ 7.93-7.88 (m, 2H), 7.38 (dd, J=7.9, 0.4 Hz, 1H), 3.95 (t, J=6.8 Hz, 2H), 3.49 (t, J=6.8 Hz, 2H), 1.38 (s, 3H), 1.37 (s, 6H); ESI MS m/z 279 [C₁₆H₁₉ClO₂+H]⁺.

### Example 95

### 5-[3-(4-BENZO[D]ISOTHIAZOL-3-YL-PIPERAZIN-1-YL)-PROPIONYL]-1,1,3,3-TETRAMETHYL-INDAN-2-ONE

A mixture of 5-(3-Chloro-propionyl)-1,1,3,3-tetramethyl-indan-2-one (4.00 g, 14.4 mmol), 3-piperazin-1-yl-benzo[d]isothiazole hydrochloride (3.81 g, 14.4 mmol), potassium carbonate (6.57 g, 47.5 mmol), sodium iodide (2.16 g, 14.4 mmol) in acetonitrile (250 mL) was stirred at rt for 24 h. The reaction was quenched with water (120 mL), and acetonitrile was evaporated. The residue was extracted with methylene chloride (2×250 mL). The combined organic extracts were washed with water, brine, dried over Na₂SO₄, evaporated. The residue was purified by chromatography (silica gel, 7:3 to 1:1 hexanes/EtOAc) to give 5-[3-(4-Benzo[d]isothiazol-3-yl-piperazin-1-yl)-propionyl]-1,1,3,3-tetramethyl-indan-2-one (4.60 g, 70%) as a pale yellow solid: ¹H NMR (300 MHz, CDCl₃) δ 7.97-7.90 (m, 3H), 7.82 (d, J=8.1 Hz, 1H), 7.47 (dd, J=7.1, 0.9 Hz, 1H), 7.39-7.33 (m, 2H), 3.58 (t, J=4.8 Hz, 4H), 3.27 (t, J=7.3 Hz, 2H), 2.97 (t, J=7.3 Hz, 2H), 2.78 (t, J=4.9 Hz, 4H), 1.38 (s, 6H), 1.37 (s, 6H); ESI MS m/z 462 [C₂₇H₃₁N₃O₂S+H]⁺.

### Preparation 24

### 5-[3-(4-BENZO[D]ISOTHIAZOL-3-YL-PIPERAZIN-1-YL)-1-HYDROXY-PROPYL]-1,1,3,3-TETRAMETHYL-INDAN-2-ONE AND 5-[3-(4-BENZO[D]ISOTHIAZOL-3-YL-PIPERAZIN-1-YL)-1-HYDROXY-PROPYL]-1,1,3,3-TETRAMETHYL-INDAN-2-OL

To a suspension of 5-[3-(4-Benzo[d]isothiazol-3-yl-piperazin-1-yl)-propionyl]-1,1,3,3-tetramethyl-indan-2-one (3.30 g, 7.16 mmol) in 2-propanol (300 mL) and methanol (300 mL) was added NaBH₄ (0.28 g, 7.5 mmol) at rt. After stirring at rt for 5 h, the reaction was quenched with the addition of acetone. The solvent was removed in vacuo. The residue was taken up in chloroform (1 L), washed with water, brine, dried over Na₂SO₄, evaporated, and chromatographed (silica gel, 97:3 to 95:5 CH₂Cl₂/MeOH) to a mixture of compounds 5-[3-(4-Benzo[d]isothiazol-3-yl-piperazin-1-yl)-propionyl]-1,1,3,3-tetramethyl-indan-2-one, 5-[3-(4-Benzo[d]isothiazol-3-yl-piperazin-1-yl)-1-hydroxy-propyl]-1,1,3,3-tetramethyl-indan-2-one and 5-[3-(4-Benzo[d]isothiazol-3-yl-piperazin-1-yl)-1-hydroxy-propyl]-1,1,3,3-tetramethyl-indan-2-ol (1.73 g, 3:10:4 by HPLC analysis). Also, 0.75 g of 5-[3-(4-Benzo[d]isothiazol-3-yl-piperazin-1-yl)-propionyl]-1,1,3,3-tetramethyl-indan-2-one was recovered.

### Examples 96-97

### 5-[3-(4-BENZO[D]ISOTHIAZOL-3-YL-PIPERAZIN-1-YL)-1-CHLORO-PROPYL]-1,1,3,3-TETRAMETHYL-INDAN-2-ONE AND 5-[3-(4-BENZO[D]ISOTHIAZOL-3-YL-PIPERAZIN-1-YL)-1-CHLORO-PROPYL]-1,1,3,3-TETRAMETHYL-INDAN-2-OL

The above mixture of 5-[3-(4-Benzo[d]isothiazol-3-yl-piperazin-1-yl)-propionyl]-1,1,3,3-tetramethyl-indan-2-one, 5-[3-(4-Benzo[d]isothiazol-3-yl-piperazin-1-yl)-1-hydroxy-propyl]-1,1,3,3-tetramethyl-indan-2-one and 5-[3-(4-Benzo[d]isothiazol-3-yl-piperazin-1-yl)-1-hydroxy-propyl]-1,1,3,3-tetramethyl-indan-2-ol (1.73 g, approximate 3.02 mmol) in CH₂Cl₂ (60 mL) was treated with methanesulfonyl chloride (0.23 mL, 3.0 mmol) and triethylamine (0.52 mL, 3.8 mmol) at 0° C. The reaction mixture was allowed to stir at 0° C. for 2 h, and then quenched with water. The organic layer was separated, washed with water, brine, dried over Na₂SO₄, and evaporated. The residue was purified by chromatography (silica gel, 4:1 to 3:2 hexanes/EtOAc) to provide compounds 5-[3-(4-Benzo[d]isothiazol-3-yl-piperazin-1-yl)-1-chloro-propyl]-1,1,3,3-tetramethyl-indan-2-one (820 mg, 58%) and 5-[3-(4-Benzo[d]isothiazol-3-yl-piperazin-1-yl)-1-chloro-propyl]-1,1,3,3-tetramethyl-indan-2-ol (360 mg, 25%).

### Example 96

### 5-[3-(4-BENZO[D]ISOTHIAZOL-3-YL-PIPERAZIN-1-YL)-1-CHLORO-PROPYL]-1,1,3,3-TETRAMETHYL-INDAN-2-ONE

¹H NMR (300 MHz, CDCl₃) δ 7.90 (d, J=8.1 Hz, 1H), 7.82 (d, J=8.1 Hz, 1H), 7.47 (td, J=7.0, 1.0 Hz, 1H), 7.38-7.33 (m, 2H), 7.30-7.25 (m, 2H), 5.10 (dd, J=8.6, 5.8 Hz,1H), 3.57 (t, J=4.9 Hz, 4H), 2.73-2.56 (m, 8H), 2.40-2.22 (m, 2H), 1.35 (s, 6H), 1.34 (s, 6H).

### Example 97

### 5-[3-(4BENZO[D]ISOTHIAZOL-3-YL-PIPERAZIN-1-YL)-1-CHLORO-PROPYL]-1,1,3,3-TETRAMETHYL-INDAN-2-OL

¹H NMR (300 MHz, CDCl₃) δ 7.90 (d, J=8.1 Hz,1H), 7.82 (d, J=8.1 Hz, 1H), 7.47 (td, J=7.0, 1.0 Hz, 1H), 7.35 (td, J=7.0, 1.0 Hz, 1H), 7.30-7.27 (m, 1H), 7.17-7.13 (m, 2H), 5.07 (dd, J=8.0, 5.9 Hz, 1H), 3.84 (d, J=7.2 Hz), 3.56 (t, J=4.8 Hz, 4H), 2.72-2.63 (m, 4H), 2.57 (t, J=7.0 Hz, 2H), 2.38-2.22 (m, 2H), 1.66 (d, J=8.3 Hz, 1H), 1.37 (s, 3H), 1.36 (s, 3H), 1.19 (s, 3H), 1.18 (s, 3H).

### Example 98

### 5-[3-(4-BENZO[D]ISOTHIAZOL-3-YL-PIPERAZIN-1-YL)-PROPYL]-1,1,3,3-TETRAMETHYL-INDAN-2-ONE METHANESULFONATE

A solution of 5-[3-(4-Benzo[d]isothiazol-3-yl-piperazin-1-yl)-1-chloro-propyl]-1,1,3,3-tetramethyl-indan-2-one (1.30 g, 2.70 mmol) in toluene (50 mL) in a sealed flask was degassed by bubbling N₂ for 5 min. The solution was then treated with tri-n-butyl tin hydride (1.10 mL, 4.00 mmol) and AIBN (66 mg, 0.40 mmol), and heated to 80° C. for 1 h. The reaction was cooled, and quenched with water. Toluene was removed in vacuo, and the residue was taken up in methylene chloride (80 mL). The organic layer was separated, washed with water, brine, dried over Na₂SO₄, and evaporated. The residue was purified by chromatography (silica gel, 6:4 hexanes/EtOAc) to give the free base of 5-[3-(4-Benzo[d]isothiazol-3-yl-piperazin-1-yl)-propyl]-1,1,3,3-tetramethyl-indan-2-one (1.13 g, 93%) as a white foam. To a solution of this free base of 5-[3-(4-Benzo[d]isothiazol-3-yl-piperazin-1-yl)-propyl]-1,1,3,3-tetramethyl-indan-2-one (610 mg, 1.36 mmol) in ethyl acetate (10 mL) was treated with CH₃SO₃H (2M in Et₂O, 0.68 mL, 1.36 mmol). The reaction mixture was stirred at rt for 20 min, while the solution remained clear. Another portion of CH₃SO₃H (3.00 mL) was added to the mixture, resulting in the inception of precipitation. Stirring was continued for 15 min. The precipitate was collected by filtration and dried in a vacuum oven at 55° C. overnight to give 5-[3-(4-Benzo[d]isothiazol-3-yl-piperazin-1-yl)-propyl]-1,1,3,3-tetramethyl-indan-2-one methanesulfonate (920 mg, 83%) as a white solid: mp 144-147° C.; ¹H NMR (300 MHz, DMSO-d₆) δ 9.67 (br s, 1H), 8.70 (br s, 2.2H), 8.15 (d, J=8.2 Hz, 1H), 8.12 (d, J=8.2 Hz,1H), 7.63-7.58 (m,1H), 7.55-7.47 (m, 1H), 7.34 (d, J=7.8 Hz, 1H), 7.29 (s, 1H), 7.21 (dd, J=1.5 Hz and 7.8 Hz, 1H), 4.10 (d, J=10.2 Hz, 2H), 3.45-3.25 (m, 8H), 2.68 (t, J=7.8 Hz, 2H), 2.41 (s, 9.6 H), 1.28 (s, 6H), 1.26 (s, 6H); ESI MS m/z 448 [C₂₇H₃₃N₃OS+H]⁺; R_{f} 0.37 (40:1 CH₂Cl₂/MeOH); HPLC >99% (AUC), t_{R}=15.62 min. Anal. Calc'd for C₂₇H₃₃N₃OS.3.2CH₃SO₃H: C, 48.03; H, 6.11; N, 5.56. Found: C, 48.25; H, 6.24; N, 5.42.

### Example 99

### 5-[3-(4-BENZO[D]ISOTHIAZOL-3-YL-PIPERAZIN-1-YL)-PROPYL]-1,1,3,3-TETRAMETHYL-INDAN-2-OL METHANESULFONATE

Starting with 5-[3-(4-Benzo[d]isothiazol-3-yl-piperazin-1-yl)-1-chloro-propyl]-1,1,3,3-tetramethyl-indan-2-ol (700 mg, 1.45 mmol) and following the procedure as outlined in Example 98, the free base of 5-[3-(4-Benzo[d]isothiazol-3-yl-piperazin-1-yl)-propyl]-1,1,3,3-tetramethyl-indan-2-ol (400 mg) was obtained as a white foam. A solution of this free base of 5-[3-(4-Benzo[d]isothiazol-3-yl-piperazin-1-yl)-propyl]-1,1,3,3-tetramethyl-indan-2-ol (400 mg, 0.890 mmol) in ethyl acetate (10 mL) was treated with CH₃SO₃H (2M in Et₂O, 0.45 mL, 0.90 mmol). The reaction mixture was stirred at rt for 20 min. The precipitate was collected by filtration and dried in a vacuum oven at 55° C. overnight to give 5-[3-(4-Benzo[d]isothiazol-3-yl-piperazin-1-yl)-propyl]-1,1,3,3-tetramethyl-indan-2-ol methanesulfonate (475 mg, 60%) as a white solid: mp 223-225° C.; ¹H NMR (300 MHz, DMSO-d₆) δ 9.60 (br s, 1H), 8.15 (d, J=9.3 Hz, 1H), 8.12 (d, J=8.7 Hz, 1H), 7.58 (m, 1H), 7.48 (dd, J=7.3 and 7.1 Hz, 1H), 7.12-7.04 (m, 3H), 4.10 (d, J=9.6 Hz, 2H), 3.66-3.21 (m, 1OH), 2.62 (t, J=7.7 Hz, 2H), 2.33 (s, 3 H), 1.18-2.10 (m, 1H), 1.27 (s, 3H), 1.25 (s, 3H), 1.07 (s, 3H), 1.05 (s, 3H); ESI MS m/z 450 [C₂₇H₃₅N₃OS+H]⁺; R_{f} 0.24 (40:1 CH₂Cl₂/MeOH); HPLC 98.0% (AUC), t_{R}=14.55 min. Anal. Calc'd for C₂₇H₃₅N₃OS.CH₃SO₃H.0.5H₂O: C, 60.62; H, 7.27; N, 7.57. Found: C, 60.88; H, 7.21; N, 7.31.

### Example 100

### 5-[3-(4-BENZO[D]ISOXAZOL-3-YL-PIPERAZIN-1-YL)-PROPIONYL]-1,1,3,3-TETRAMETHYL-INDAN-2-ONE

A mixture of 5-(3-Chloro-propionyl)-1,1,3,3-tetramethyl-indan-2-one (6.00 g, 21.6 mmol), 3-piperazin-1-yl-benzo[d]isoxazole hydrochloride (6.20 g, 21.9 mmol), potassium carbonate (9.84 g, 71.2 mmol), sodium iodide (3.21 g, 21.6 mmol) in acetonitrile (400 mL) was heated to reflux for 4 h. The reaction was diluted with water (200 mL), and acetonitrile was evaporated. The residue was extracted with ethyl acetate (2×250 mL). The combined organic extracts were washed with water, brine, dried over Na₂SO₄, evaporated. The residue was purified by chromatography (silica gel, 1:1 hexanes/EtOAc) to give 5-[3-(4-Benzo[d]isoxazol-3-yl-piperazin-1-yl)-propionyl]-1,1,3,3-tetramethyl-indan-2-one (4.00 g, 42%) as a pale yellow solid: ¹H NMR (300 MHz, CDCl₃) δ 7.94 (dd, J=8.0, 1.6 Hz, 1H), 7.90 (d, J=1.3 Hz, 1H), 7.70 (d, J=8.1 Hz, 1H), 7.52-7.44 (m, 2H), 7.38 6(d, J=7.9 Hz, 1H), 7.24-7.20 (m, 1H), 3.61 (t, J=5.0 Hz, 4H), 3.26 (t, J=7.5 Hz 2H), 2.94 (t, J=7.5 Hz, 2H), 2.75 (t, J=5.0 Hz, 4H), 1.38 (s, 6H), 1.37 (s, 6H).

### Example 101

### 5-[3-(4BENZO[D]ISOXAZOL-3-YL-PIPERAZIN-1-YL)-1-HYDROXY-PROPYL]-1,1,3,3-TETRAMETHYL-INDAN-2-ONE

A suspension of 5-[3-(4-Benzo[d]isoxazol-3-yl-piperazin-1-yl)-propionyl]-1,1,3,3-tetramethyl-indan-2-one (4.00 g, 8.98 mmol) in 2-propanol (250 mL) and methanol (250 mL) was warmed while stirring until a clear solution was formed. The mixture was then cooled to 0° C., and treated with NaBH₄ (0.37 g, 9.9 mmol). After stirring at 0° C. for 7 h, the reaction was quenched with the addition of acetone. The solvent was removed in vacuo. The residue was taken up in methylene chloride (500 mL), washed with water, brine, dried over Na₂SO₄, evaporated, and chromatographed (silica gel, 2:3 hexanes/EtOAc) to give 5-[3-(4-Benzo[d]isoxazol-3-yl-piperazin-1-yl)-1-hydroxy-propyl]-1,1,3,3-tetramethyl-indan-2-one (3.00 g, 75%) as a white solid: ¹H NMR (300 MHz, CDCl₃) δ 7.69 (d, J=8.1 Hz, 1H), 7.53-7.45 (m, 2H), 7.32-7.14 (m, 4H), 6.29 (br s, 1H), 4.99 (dd, J=8.6, 4.0 Hz, 1H), 3.85-3.62 (m, 4H), 2.91-2.66 (m, 6H), 1.99-1.90 (m, 2H), 1.35 (s, 6H), 1.34 (s, 6H).

### Example 102

### 5-[3-(4-BENZO[D]ISOXAZOL-3-YL-PIPERAZIN-1-YL)-1-CHLORO-PROPYL]-1,1,3,3-TETRAMETHYL-INDAN-2-ONE

A solution of 5-[3-(4-Benzo[d]isoxazol-3-yl-piperazin-1-yl)-1-hydroxy-propyl]-1,1,3,3-tetramethyl-indan-2-one (3.00 g, 6.70 mmol) in CH₂Cl₂ (70 mL) was treated with methanesulfonyl chloride (0.67 mL, 8.7 mmol) and triethylamine (1.49 mL, 10.7 mmol) at 0° C. The reaction mixture was allowed to stir at rt for 1 h, and then quenched with water. The organic layer was separated, washed with water, brine, dried over Na₂SO₄, and evaporated. The residue was purified by chromatography (silica gel, 7:3 to 3:2 hexanes/EtOAc) to provide 5-[3-(4-Benzo[d]isoxazol-3-yl-piperazin-1-yl)-1-chloro-propyl]-1,1,3,3-tetramethyl-indan-2-one (2.10 g, 68%) as a white foam: ¹H NMR (300 MHz, CDCl₃) δ 7.69 (d, J=8.1 Hz, 1H), 7.52-7.44 (m, 2H), 7.36 (dd, J=7.8, 1.7 Hz, 1H), 7.29-7.19 (m, 3H), 5.09 (dd, J=8.6, 5.7 Hz, 1H), 3.59 (t, J=5.0 Hz, 4H), 2.70-2.63 (m, 4H), 2.61-2.55 (m, 2H), 2.38-2.21 (m, 2H), 1.35 (s, 6H), 1.34 (s, 6H); ESI MS m/z 466 [C₂₇H₃₂ClN₃O₂+H]⁺.

### Example 103

### 5-[3-(4-BENZO[D]ISOXAZOL-3-YL-PIPERAZIN-1-YL)-PROPYL]-1,1,3,3-TETRAMETHYL-INDAN-2-ONE METHANESULFONATE

A solution of 5-[3-(4-Benzo[d]isoxazol-3-yl-piperazin-1-yl)-1-chloro-propyl]-1,1,3,3-tetramethyl-indan-2-one (2.10 g, 4.51 mmol) in toluene (75 mL) in a sealed flask was degassed by bubbling N₂ for 5 min. The solution was then treated with tri-n-butyl tin hydride (1.82 mL, 6.77 mmol) and AIBN (112 mg, 0.680 mmol), and heated to 80° C. for 1.5 h. The reaction was cooled, and quenched with water. Toluene was removed in vacuo, and the residue was taken up in methylene chloride (100 mL). The organic layer was separated, washed with water, brine, dried over Na₂SO₄, and evaporated. The residue was purified by chromatography (silica gel, 6:4 hexanes/EtOAc) to give the free base of 5-[3-(4-Benzo[d]isoxazol-3-yl-piperazin-1-yl)-propyl]-1,1,3,3-tetramethyl-indan-2-one (1.80 g, 95%) as a white semi-solid. A solution of 5-[3-(4-Benzo[d]isoxazol-3-yl-piperazin-1-yl)-propyl-1,1,3,3-tetramethyl-indan-2-one (free base, 550 mg, 1.27 mmol) in ethyl acetate (10 mL) was treated with CH₃SO₃H (2M in Et₂O, 0.67 mL, 1.3 mmol). The reaction mixture was stirred at rt for 15 min. The precipitate was collected by filtration and dried in a vacuum oven at 55° C. overnight to give compound 5-[3-(4-Benzo[d]isoxazol-3-yl-piperazin-1-yl)-propyl]-1,1,3,3-tetramethyl-indan-2-one methanesulfonate (540 mg, 81%) as a white solid: mp 200-203° C. dec; ¹H NMR (300 MHz, DMSO-d₆) [symbol] 9.73 (br s, 1H), 8.06 (d, J=8.1 Hz, 1H), 7.68-7.60 (m, 2H), 7.38-7.33 (m, 2H), 7.28 (s, 1H), 7.21 (dd, J=7.8, 1.4 Hz, 1H), 4.16 (d, J=12.8 Hz, 2H), 3.66 (d, J=11.5 Hz, 2H), 3.44-3.28 (m, 6H), 2.68 (t, J=7.8 Hz, 2H), 2.35 (s, 3H), 2.35-1.99 (m, 2H), 1.27 (s, 6H), 1.26 (s, 6H); ESI MS m/z 432 [C₂₇H₃₃N₃O₂+H]⁺; R_{f} 0.20 (3:2 Hexanes/EtOAc); HPLC >99% (AUC), t_{R}=15.15 min. Anal. Calc'd for C₂₇H₃₃N₃O₂.CH₃SO₃H: C, 63.73; H, 7.07; N, 7.96. Found: C, 63.57; H, 7.20; N, 7.90.

### Example 104

### 5-[3-(4-BENZO[D]ISOXAZOL-3-YL-PIPERAZIN-1-YL)-PROPYL]-1,1,3,3-TETRAMETHYL-INDAN-2-OL METHANESULFONATE

To a suspension of compound 5-[3-(4-Benzo[d]isoxazol-3-yl-piperazin-1-yl)-propyl]-1,1,3,3-tetramethyl-indan-2-one (free base, 600 mg, 1.39 mmol) in 2-propanol (30 mL) and methanol (30 mL) was added NaBH₄ (105 mg, 2.78 mmol) at 0° C. The reaction mixture was allowed to stir at rt overnight, and was then quenched with the addition of acetone. The solvent was removed in vacuo. The residue was taken up in methylene chloride (100 mL), washed with water, brine, dried over Na₂SO₄, evaporated, and chromatographed (silica gel, 93:3 CH₂Cl₂/MeOH) to provide the free base of 5-[3-(4-Benzo[d]isoxazol-3-yl-piperazin-1-yl)-propyl]-1,1,3,3-tetramethyl-indan-2-ol (510 mg, 85%) as a floppy white solid. 5-[3-(4-Benzo[d]isoxazol-3-yl-piperazin-1-yl)-propyl]-1,1,3,3-tetramethyl-indan-2-ol (free base, 510 mg, 1.18 mmol) was dissolved in ethyl acetate (10 mL), and then treated with CH₃SO₃H (2M in Et₂O, 0.62 mL, 1.22 mmol). The reaction mixture was stirred at rt for 10 min. The precipitate was collected by filtration and dried in a vacuum oven at 55° C. overnight to give 5-[3-(4-Benzo[d]isoxazol-3-yl-piperazin-1-yl)-propyl]-1,1,3,3-tetramethyl-indan-2-ol methanesulfonate (601 mg, 95%) as a white solid: mp 240-242° C.; ¹H NMR (300 MHz, DMSO-d₆) [symbol] 9.65 (br s, 1H), 8.05 (d, J=8.1 Hz, 1H), 7.66-7.60 (m, 2H), 7.38-7.33 (m, 1H), 7.11-7.04 (m, 3H), 5.05 (br s, 1H), 4.15 (d, J=12.6 Hz, 2H), 3.67-3.64 (m, 3H), 3.45-3.23 (m, 6H), 2.61 (t, J=7.7 Hz, 2H), 2.33 (s, 3H), 2.05-1.91 (m, 2H), 1.26 (s, 3H), 1.24 (s, 3H), 1.06 (s, 3H), 1.04 (s, 3H); ESI MS m/z 434 [C₂₇H₃₅N₃O₂+H]⁺; R_{f} 0.19 (2:3 EtOAc/hexanes); HPLC 98.8% (AUC), t_{R}=13.82 min. Anal. Calc'd for C₂₇H₃₅N₃O₂.CH₃SO₃H.0.5H₂O: C, 62.43; H, 7.48; N, 7.80. Found: C, 62.62; H, 7.45; N, 7.67.

### Example 105

### {5-[2-(4-BENZO[D]ISOTHIAZOL-3-YL-PIPERAZIN-1-YL)-ETHYL]-1,1,3,3-TETRAMETHYL-INDAN-2-YL}-METHYLAMINE METHANESULFONATE

Into a sealed tube was placed 5-[2-(4-Benzo[d]isothiazol-3-yl-piperazin-1-yl)-ethyl]-1,1,3,3-tetramethyl-indan-2-one (500 mg, 1.15 mmol) and methylamine (2.0 M in THF, 8.63 mL, 17.2 mmol). The mixture was cooled to 0° C., and titanium tetrachloride (2M in CH₂Cl₂, 4.61 mL, 9.22 mmol) was then added. The mixture was diluted with toluene (15 mL) and heated to 150° C. for 2 h. After cooling down, the reaction mixture was diluted with methylene chloride (100 mL). The solid was filtered off via a pad of Celite®. The filtrate was concentrated to give a brown solid (670 mg, theoretical 513 mg) that was dissolved in a solution of 2-propanol (20 mL) and methanol (20 mL). The solution was then treated with NaBH₄ (284 mg, 7.50 mmol) at rt. The reaction mixture was allowed to stir at rt overnight, and was then quenched with the addition of acetone. The solvent was removed in vacuo. The residue was taken up in methylene chloride (150 mL), washed with water, brine, dried over Na₂SO₄, evaporated, and chromatographed (silica gel, 20:1 CH₂Cl₂/MeOH) to provide the free base of {5-[2-(4-Benzo[d]isothiazol-3-yl-piperazin-1-yl)-ethyl]-1,1,3,3-tetramethyl-indan-2-yl}-methylamine (420 mg) as a semi-solid. This free base of {5-[2-(4-Benzo[d]isothiazol-3-yl-piperazin-1-yl)-ethyl]-1,1,3,3-tetramethyl-indan-2-yl}-methylamine (410 mg, 0.910 mmol) was dissolved in ethyl acetate (8 mL), and then treated with CH₃SO₃H (2M in Et₂O, 0.48 mL, 0.96 mmol). The reaction mixture was stirred at rt for 30 min. The precipitate was collected by filtration and dried in a vacuum oven at 55° C. overnight to give {5-[2-(4-Benzo[d]isothiazol-3-yl-piperazin-1-yl)-ethyl]-1,1,3,3-tetramethyl-indan-2-yl}-methylamine methanesulfonate (318 mg, 64%) as a white solid: mp 200-203° C. dec; ¹H NMR (300 MHz, CDCl₃) [symbol] 7.91 (d, J=8.1 Hz, 1H), 7.83 (d, J=8.1 Hz, 1H), 7.48 (td, J=7.1, 0.8 Hz, 1H), 7.37 (td, J=7.1, 0.8 Hz, 1H), 7.15 (dd, J=7.8, 1.2 Hz, 1H), 7.08 (d, J=7.8 Hz, 1H), 7.01 (s, 1H), 3.68 (m, 4H), 3.16 (s, 1H), 3.03 (s, 3H), 2.95-2.77 (m, 12H), 1.56 (s, 3H), 1.55 (s, 3H), 1.40 (s, 3H), 1.39 (s, 3H); ESI MS m/z 449 [C₂₇H₃₆N₄S+H]⁺; R_{f} 0.23 (20:1 CH₂Cl₂/MeOH); HPLC >99% (AUC), t_{R}=10.99 min. Anal. Calc'd for C₂₇H₃₆N₄S.CH₃SO₃H.0.25H₂O: C, 61.23; H, 7.43; N, 10.20. Found: C, 61.25; H, 7.42; N, 10.09.

### Example 106

### N-{5-[2-(4BENZO[D]ISOTHIAZOL-3-YL-PIPERAZIN-1-YL)-ETHYL]-1,1,3,3-TETRAMETHYL-INDAN-2-YL}-N-METHYLACETAMIDE METHANESULFONATE

To a solution of {5-[2-(4-Benzo[d]isothiazol-3-yl-piperazin-1-yl)-ethyl]-1,1,3,3-tetramethyl-indan-2-yl}-methylamine (free base, 900 mg, 2.00 mmol) in methylene chloride (20 mL) was added acetic anhydride (0.75 mL, 8.0 mmol) and triethylamine (1.67 mL, 6.00 mmol). The mixture was stirred at rt for 5 h, and quenched with water. The organic phase was separated, washed with water, brine, dried over Na₂SO₄, evaporated, and chromatographed (silica gel, 25:1 CH₂Cl₂/MeOH with 0.5% Et₃N) to provide the free base of N-{5-[2-(4-Benzo[d]isothiazol-3-yl-piperazin-1-yl)-ethyl]-1,1,3,3-tetramethyl-indan-2-yl}-N-methylacetamide (800 mg, 82%) as a pale yellow solid. N-{5-[2-(4-Benzo[d]isothiazol-3-yl-piperazin-1-yl)-ethyl]-1,1,3,3-tetramethyl-indan-2-yl}-N-methylacetamide (free base, 792 mg, 1.61 mmol) was dissolved in ethyl acetate (10 mL), and then treated with CH₃SO₃H (2M in Et₂O, 0.81 mL, 1.62 mmol). The reaction mixture was stirred at rt for 15 min. The precipitate was collected by filtration and dried in a vacuum oven at 55° C. overnight to give N-(5-[2-(4-Benzo[d]isothiazol-3-yl-piperazin-1-yl)-ethyl]-1,1,3,3-tetramethyl-indan-2-yl}-N-methylacetamide methanesulfonate (809 mg, 87%) as a white solid: mp 248-250° C.; ¹H NMR (300 MHz, CDCl₃, Note: mixture of two rotamers) [symbol] 11.60 (br s, 1H), 7.88-7.83 (m, 2H), 7.53 (t, J=7.5 Hz, 1H), 7.42 (t, J=7.5 Hz, 1H), 7.18-6.99 (m, 3H), 5.13 (s, 0.5 H), 4.19-3.96 (m, 4.5 H), 3.70-3.67 (m, 2H), 3.33-3.13 (m, 6H), 2.90 (s, 3H), 2,71 (s, 1.5H), 2.67 (s, 1.5H), 2.22 (s, 1.5 H), 2.12 (s, 1.5 H), 1.42 (s, 1.5H), 1.41 (s, 1.5H), 1.40 (s, 1.5H), 1.39 (s, 1.5H), 1.33 (s, 1.5H), 1.32 (s, 1.5H), 1.29 (s, 1.5H), 1.28 (s, 1.5H); ESI MS m/z 491 [C₂₉H₃₈N₄OS+H]⁺; R_{f} 0.45 (20:1 CH₂Cl₂/MeOH); HPLC >98.1% (AUC), t_{R}=14.00 min. Anal. Calc'd for C₂₉H₃₈N₄OS.CH₃SO₃H: C, 61.40; H, 7.21; N, 9.55. Found: C, 61.28; H, 7.31; N, 9.52.

### Example 107

### 5-[2-(4-BENZO[D]ISOTHIAZOL-3-YL-PIPERAZIN-1-YL)-ETHYL-1,1,3,3-TETRAMETHYL-INDAN-2-YLIDENEAMINE

A solution of 5-[2-(4-Benzo[d]isothiazol-3-yl-piperazin-1-yl)-ethyl]-1,1,3,3-tetramethyl-indan-2-one (3.20 g, 7.66 mmol,), hydroxylamine hydrochloride (1.60 g, 23.0 mmol) in pyridine (100 mL) was heated to 100° C. for 5 h, then allowed to cool. The solvent was removed in vacuo, and the residue was partitioned between CH₂Cl₂ (300 mL) and water (100 mL). The organic layer was separated, washed with water, brine, dried over Na₂SO₄, filtered, and concentrated in vacuo to provide the oxime (2.75 g, 80%) as a white solid: ESI MS m/z 449 [C₂₆H₃₂N₄OS+H]⁺. The white solid (2.75 g, 6.14 mmol) was dissolved in dioxane (60 mL). The solution was treated titanium trichloride (26.5 mL, ˜8.9 wt % in 30% HCl, 18.4 mmol), and the reaction was allowed to stirred under N₂ at rt overnight. The reaction mixture was basified with 5.8 N NaOH (75 mL), diluted with water (100 mL) and extracted with methylene chloride (300 mL once, then 2×100 mL). The combined organic extracts were washed with water, brine, dried over Na₂SO₄, filtered, and concentrated in vacuo. The crude residue was purified by chromatography on silica gel (1:1 to 4:1 EtOAc/hexanes) to provide 5-[2-(4-Benzo[d]isothiazol-3-yl-piperazin-1-yl)-ethyl]-1,1,3,3-tetramethyl-indan-2-ylideneamine (1.50 g, 57%) as a white foam: ¹H NMR (300 MHz, CDCl₃) δ 9.40 (br s, 1H), 7.93 (d, J=8.1 Hz, 1H), 7.92 (d, J=8.1 Hz, 1H), 7.50-7.45 (m, 1H), 7.39-7.34 (m, 1H), 7.20-7.12 (m, 2H), 7.10 (s, 1H), 3.63-3.60 (m, 4H), 2.92-2.70 (m, 8H), 1.38 (s, 12H); ESI MS m/z433 [C₂₆H₃₂N₄S +H]⁺.

### Example 108

### 5-[2-(4-BENZO[D]ISOTHIAZOL-3-YL-PIPERAZIN-1-YL)-ETHYL]-1,1,3,3-TETRAMETHYL-INDAN-2-YLAMINE

To a solution of 5-[2-(4-Benzo[d]isothiazol-3-yl-piperazin-1-yl)-ethyl]-1,1,3,3-tetramethyl-indan-2-ylideneamine (1.25 g, 2.89 mmol) in 2-propanol (20 mL) and methanol (20 mL) was added NaBH₄ (328 mg, 8.68 mmol) at 0° C. The reaction mixture was allowed to stir at rt for 16 h, and was then quenched with the addition of acetone. The solvent was removed in vacuo. The residue was taken up in methylene chloride (250 mL), washed with water, brine, dried over Na₂SO₄, evaporated, and chromatographed (silica gel, 95:5 CH₂Cl₂/MeOH) to provide 5-[2-(4-Benzo[d]isothiazol-3-yl-piperazin-1-yl)-ethyl]-1,1,3,3-tetramethyl-indan-2-ylamine (1.12 g, 90%) as a pale yellow semi-solid: ¹H NMR (300 MHz, CDCl₃) δ 7.93 (d, J=8.1 Hz, 1H), 7.82 (d, J=8.1 Hz, 1H), 7.47 (td, J=7.0, 1.0 Hz, 1H), 7.36 (td, J=7.0, 1.0 Hz, 1H), 7.14-7.08 (m, 2H), 7.03 (s, 1H), 3.63-3.60 (m, 4H), 2.94-2.68 (m, 8H), 2.0-1.0 (br s, 2H), 1.32 (s, 3H), 1.31 (s, 3H), 1.11 (s, 3H) 1.10 (s, 3H); ESI MS m/z 435 [C₂₆H₃₄N₄S+H]⁺.

### Example 109

### N-{5-[2-(4-BENZO[D]ISOTHIAZOL-3-YL-PIPERAZIN-1-YL)-ETHYL]-1,1,3,3-TETRAMETHYL-INDAN-2-YL}-ACETAMIDE

To a solution of 5-[2-(4-Benzo[d]isothiazol-3-yl-piperazin-1-yl)-ethyl]-1,1,3,3-tetramethyl-indan-2-ylamine (595 mg, 1.14 mmol) in methylene chloride (20 mL) was added acetic anhydride (0.21 mL, 2.3 mmol) and triethylamine (0.63 mL, 4.5 mmol). The mixture was stirred at rt for 2 h, and quenched with water. The organic phase was separated, washed with water, brine, dried over Na₂SO₄, evaporated, and chromatographed (silica gel, 20:1 CH₂Cl₂/MeOH) to provide N-{5-[2-(4-Benzo[d]isothiazol-3-yl-piperazin-1-yl)-ethyl]-1,1,3,3-tetramethyl-indan-2-yl}-acetamide (440 mg, 81%) as a white solid: mp 75-80° C.; ¹0H NMR (300 MHz, CDCl₃) δ 8.01(d, J=8.1 Hz, 1H), 7.90 (d, J=8.1 Hz, 1H), 7.58-7.53 (m, 1H), 7.47-7.42 (m, 1H), 7.21-7.16 (m, 2H), 7.10 (s, 1H), 5.63 (d, J=10.7 Hz, 1H), 4.50 (d, J=10.7 Hz), 3.72-3.68 (m, 4H), 2.98-2.77 (m, 1OH), 2.22 (s, 3H), 1.44 (s, 3H), 1.43 (s, 3H), 1.23 (s, 3H), 1.22 (s, 3H); ESI MS m/z 477 [C₂₈H₃₆N₄OS+H]⁺; R_{f} 0.44 (20:1 CH₂Cl₂/MeOH); HPLC 96.2% (AUC), t_{R}=13.47 min. Anal. Calc'd for C₂₈H₃₆N₄OS.0.5H₂O: C, 69.24; H, 7.68; N, 11.54. Found: C, 68.90; H, 7.92; N, 11.37.

### Example 110

### 5-[2-(4-BENZO[D]ISOXAZOL-3-YL-PIPERAZIN-1-YL)-ETHYL]-1,1,3,3-TETRAMETHYL-INDAN-2-ONE OXIME

A solution of 5-[2-(4-Benzo[d]isoxazol-3-yl-piperazin-1-yl)-1-chloro-ethyl]-1,1,3,3-tetramethyl-indan-2-one (2.30 g, 5.51 mmol,), hydroxylamine hydrochloride (1.15 g, 16.5 mmol) in pyridine (80 mL) was heated to 100° C. for 6 h, then allowed to cool. The solvent was removed in vacuo, and the residue was partitioned between CH₂Cl₂ (200 mL) and water (50 mL). The organic layer was separated, washed with water, brine, dried over Na₂SO₄, filtered, and concentrated in vacuo to provide 5-[2-(4-Benzo[d]isoxazol-3-yl-piperazin-1-yl)-ethyl]-1,1,3,3-tetramethyl-indan-2-one oxime (2.30 g, 97%) as a white solid: ¹H NMR (300 MHz, CDCl₃, note: mixture of E/Z isomers) δ 7.74-7.70 (m, 2H), 7.49-7.46 (m, 2H), 7.25-7.30 (m, 3H), 3.68-3.58 (m, 4H), 2.88-2.84 (m, 2H), 2.78-2.70 (m, 6H), 1.65 (s, 3H), 1.64 (s, 3H), 1.45 (s, 3H), 1.44 (s, 3H).

### Example 111

### 5-[2-(4-BENZO[D]ISOXAZOL-3-YL-PIPERAZIN-1-YL)-ETHYL-1,1,3,3-TETRAMETHYL-INDAN-2-YLIDENEAMINE

A solution of 5-[2-(4-Benzo[d]isoxazol-3-yl-piperazin-1-yl)-ethyl]-1,1,3,3-tetramethyl-indan-2-one oxime (2.30 g, 5.32 mmol) in dioxane (120 mL) under N₂ was treated titanium(III) trichloride (23 mL, ˜8.9 wt % in 30% HCl, 16 mmol), and the reaction was allowed to stirred at rt overnight. The reaction mixture was basified with 5.8 N NaOH (45 mL), diluted with water (60 mL) and extracted with methylene chloride (3×150 mL). The combined organic extracts were washed with water, brine, dried over Na₂SO₄, filtered, and concentrated in vacuo to provide 5-[2-(4-Benzo[d]isoxazol-3-yl-piperazin-1-yl)-ethyl]-1,1,3,3-tetramethyl-indan-2-ylideneamine (2.20 g, 99% crude) as a tan solid. The crude 5-[2-(4-Benzo[d]isoxazol-3-yl-piperazin-1-yl)-ethyl]-1,1,3,3-tetramethyl-indan-2-ylideneamine was used for the next step without further purification. The analytical sample was obtained by purification of 5-[2-(4-Benzo[d]isoxazol-3-yl-piperazin-1-yl)-ethyl]-1,1,3,3-tetramethyl-indan-2-ylideneamine by chromatography (silica gel, 4:1 EtOAc/hexanes with 0.5% Et₃N) as a thick tan oil: ¹H NMR (300 MHz, CDCl₃) δ 9.35 (br s,1H), 7.71 (d, J=8.0 Hz, 1H), 7.49-7.45 (m, 1H), 7.25-7.09 (m, 4H), 3.64 (t, J=5.0 Hz4H), 2.90-2.85 (m, 2H), 2.77-2.67 (m, 6H), 1.39 (m, 6H), 1.38 (s, 6H); ESI MS m/z 417 [C₂₆H₃₂N₄O+H]⁺.

### Example 112

### 5-[2-4-BENZO[D]ISOXAZOL-3-YL-PIPERAZIN-1-YL)-ETHYL]-1,1,3,3-TETRAMETHYL-INDAN-2-YLAMINE

To a solution of 5-[2-(4-Benzo[d]isoxazol-3-yl-piperazin-1-yl)-ethyl]-1,1,3,3-tetramethyl-indan-2-ylideneamine (2.15 g, 5.17 mmol) in 2-propanol (45 mL) and methanol (45 mL) was added NaBH₄ (586 mg, 15.5 mmol) at rt. The reaction mixture was allowed to stir at rt overnight, and was then quenched with the addition of acetone. The solvent was removed in vacuo. The residue was taken up in CH₂Cl₂ (200 mL). The organic solution was washed with water, brine, dried over Na₂SO₄, and evaporated. The residue was purified by chromatography (silica gel, gradient from 4 to 5% MeOH/CH₂Cl₂) to provide 5-[2-(4-Benzo[d]isoxazol-3-yl-piperazin-1-yl)-ethyl]-1,1,3,3-tetramethyl-indan-2-ylamine (1.40 g, 65%) as a white foam: ¹H NMR (300 MHz, CDCl₃) δ 7.71 (d, J=8.0 Hz, 1H), 7.49-7.47 (m, 2H), 7.25-7.20 (m, 1H), 7.12-7.08 (m, 2H), 7.03 (s, 1H), 3.65-3.62 (m, 4H), 2.94 (s, 1H), 2.88-2.66 (m, 8H), 1.52 (br s, 2H), 1.32 (s, 3H), 1.31 (s, 3H), 1.11 (s, 3H), 1.10 (s, 3H).

### Example 113

### N-{5-[2-(4-BENZO[D]ISOXAZOL-3-YL-PIPERAZIN-1-YL)-ETHYL]-1,1,3,3-TETRAMETHYL-INDAN-2-YL}-ACETAMIDE METHANESULFONATE

To a solution of 5-[2-(4-Benzo[d]isoxazol-3-yl-piperazin-1-yl)-ethyl]-1,1,3,3-tetramethyl-indan-2-ylamine (700 mg, 1.67 mmol) in methylene chloride (20 mL) was added acetic anhydride (0.31 mL, 3.3 mmol) and triethylamine (0.93 mL, 6.7 mmol). The mixture was stirred at rt for 1 h, and quenched with water. The organic phase was separated, washed with water, brine, dried over Na₂SO₄, evaporated, and chromatographed [silica gel (Biotage), gradient from 50% to 100% EtOAC/hexanes] to provide the free base of N-{5-[2-(4-Benzo[d]isoxazol-3-yl-piperazin-1-yl)-ethyl]-1,1,3,3-tetramethyl-indan-2-yl}-acetamide (430 mg, 56%) as a white solid. The free base of N-{5-[2-(4-Benzo[d]isoxazol-3-yl-piperazin-1-yl)-ethyl]-1,1,3,3-tetramethyl-indan-2-yl}-acetamide (430 mg, 0.93 mmol) was dissolved in ethyl acetate (6 mL), and then treated with CH₃SO₃H (2M in Et₂O, 0.51 mL, 1.02 mmol). The reaction mixture was stirred at rt for 15 min, resulting in a gummy precipitate. The mixture was continued to stir for another 30 min. The precipitate was collected by filtration and dried in a vacuum oven at 55° C. overnight to give N-{5-[2-(4-Benzo[d]isoxazol-3-yl-piperazin-1-yl)-ethyl]-1,1,3,3-tetramethyl-indan-2-yl}-acetamide methanesulfonate (400 mg, 70%) as a white solid: mp 197-205° C.; ¹H NMR (300 MHz, DMSO-d₆) [symbol] 9.90 (br s, 1H), 8.08 (d, J=8.1 Hz, 1H), 7.65-7.60 (m, 3H), 7.38-7.34 (m, 1H), 7.20-7.12 (m, 3H), 4.28-4.18 (m, 3H), 3.72 (d, J=10.9 Hz, 2H), 3.46-3.28 (m, 6H), 3.05-2.99 (m, 2H), 2.37 (s, 4.8 H), 2.00 (s, 3H), 1.21 (s, 3H), 1.19 (s, 3H), 1.09 (s, 3H), 1.08 (s, 3H); ESI MS m/z 461 [C₂₈H₃₆N₄O₂+H]⁺; R_{f} 0.40 (20:1 CH₂Cl₂/MeOH); HPLC >99% (AUC), t_{R}=12.86 min. Anal. Calc'd for C₂₈H₃₆N₄O₂.1.6CH₃SO₃H: C, 57.87; H, 6.96; N, 9.12. Found: C, 58.01; H, 7.11; N, 9.01.

### Example 114

### N-{5-[2-(4-BENZO[D]ISOXAZOL-3-YL-PIPERAZIN-1-YL)-ETHYL-1,1,3,3-TETRAMETHYL-INDAN-2-YL}-METHANESULFONAMIDE

To a solution of 5-[2-(4-Benzo[d]isoxazol-3-yl-piperazin-1-yl)-ethyl]-1,1,3,3-tetramethyl-indan-2-ylamine (330 mg, 0.789 mmol) in methylene chloride (8 mL) was added methanesulfonyl chloride (0.12 mL, 1.6 mmol) and triethylamine (0.44 mL, 3.2 mmol) at 0° C. The mixture was stirred at rt for 1 h, and quenched with water. The mixture was diluted with methylene chloride (22 mL). The organic phase was separated, washed with brine, dried over Na₂SO₄, evaporated, and chromatographed (silica gel, 2:3 to 3:2 EtOAC/hexanes) to provide N-{5-[2-(4-Benzo[d]isoxazol-3-yl-piperazin-1-yl)-ethyl]-1,1,3,3-tetramethyl-indan-2-yl}-methanesulfonamide (360 mg, 92%) as a white solid: mp 75-80° C.; ¹H NMR (300 MHz, CDCl₃) δ 7.71 (d, J=8.1 Hz, 1H), 7.49-7.46 (m, 2H), 7.25-7.19 (m, 1H), 7.13-7.09 (m, 2H), 7.02 (s, 1H), 4.61 (d, J=10.8 Hz, 1H), 3.70-3.61 (m, 5H), 3.12 (s, 3H), 2.88-2.66 (m, 8H), 1.41 (s, 3H), 1.40 (s, 3H), 1.17 (s, 3H), 1.16 (s, 3H); ESI MS m/z 497 [C₂₇H₃₆N₄O₃S+H]⁺; R_{f} 0.32 (3:2 EtOAC/hexanes); HPLC 97.0% (AUC), t_{R}=13.67 min. Anal. Calc'd for C₂₇H₃₆N₄O₃S.0.5H₂O: C, 64.13; H, 7.38; N, 11.08. Found: C, 64.27; H, 7.43; N, 10.86.

### Preparation 25

### 3-METHYL-3-PHENYL-BUTYRIC ACID

To a 12 L 4-neck flask, equipped with a mechanical stirrer, condenser, and N2 inlet, was charged via cannula 3-methyl-3-phenylpropylmagnesium chloride (0.5M solution in ether) (1.6 mol, 3.2 L) and the solution was cooled in a dry ice/acetone bath. Excess solid dry ice was added portion wise over 0.5 h, with formation of a light tan solution. The reaction mixture was allowed to warm to 0° C., and the reaction mixture was quenched with cold aq HCl (200 ml conc. HCl diluted to 1 L) while cooling with an ice water bath. The clear biphasic mixture was stirred 1 h. The organic phase was separated and the ether removed on a rotovap. Aq NaOH (256 g of 50% NaOH diluted to 1 L) was added to the residual oil, and the aqueous mixture extracted twice with heptane. The heptane solution was back extracted with aq NaOH (144 g of 50% NaOH diluted to 1 L). The combined NaOH solutions were acidified with cold conc. HCl to give an oil that crystallized after several minutes. The white solid was collected and air-dried overnight. The crude acid was dissolved in EtOAc and the solution was washed with brine, dried (NaSO4) and the solvent removed on a rotovap. The residual oil crystallized on seeding to give 3-methyl-3-phenylbutyric acid as a white solid in quantitative yield. MP 53-54° C.

### Preparation 26

### 3,3-DIMETHYL-INDAN-1-ONE

To a 5 L flask was charged 3-methyl-3-phenylbutyric acid (343 g, 1.93 mole) followed by methane sulfonic acid (1.5 L). The reaction solution was heated slowly to 110° C. for 1.25 h. The heat mantle was turned off, and the brown reaction solution was allowed to cool slowly to rt overnight. The reaction mixture was poured into 4.5 L ice water with stirring. The aqueous mixture was extracted with heptane (2.5 L) followed with EtOAc (2×2 L). The combined organic solution was washed with water (2×2 L), sat'd NaHCO3 (2 L), brine, then dried (NaSO4) and the solvent removed on a rotovap to give 3,3-Dimethyl-indan-1-one (260 g) as a light brown oil. ¹H NMR (300 MHz, CDCl₃) [symbol] 7.7 (d, 1H) 7.59 (t, 1H) 7.45 (d, 1H) 7.30 (t,1H) 2.59 (s, 2H) 1.4 (s, 6H).

### Preparation 27

### 3,3-DIMETHYL-INDAN-1-OL

To a 3 L flask was added a solution of NaOH (4.0 g of 50% in 225 ml water) followed by NaBH4 (0.444 mole, 16.8 g) and the reaction mixture cooled to ˜4° C. A solution of 3,3-Dimethyl-indan-1-one (129 g, 0.81 mole) in 500 ml of EtOH was added over 1 h while maintaining the temperature at <5° C. When the addition was complete, the reaction mixture was stirred 1 h at <5° C., then allowed to warm to rt (1-2 ml of 50% NaOH were added periodically during the course of the reaction to maintain pH>9). After 2 h, TLC and LCMS showed the reaction was complete. The reaction mixture was poured into 1.5 L ice water and the aqueous mixture extracted with EtOAc (3×600 ml). The organic solution was washed with water (2×1 L), brine, then dried (NaSO4) and the solvent evaporated to give 3,3-Dimethyl-indan-1-ol (127 g) as a pale yellow brown oil. ¹H NMR (300 MHz, CDCl₃) [symbol] 7.35 (d, 1H) 7.1-7.30 (m, 3H) 5.15-5.25 (m, 1H) 2.30 (dd, 1H) 1.9 (bs, 1H) 1.79 (dd, 1H) 1.4 (s, 3H) 1.2 (s, 3H).

### Preparation 28

### 1,1-DIMETHYL-1H-INDENE

In a Kugelrohr distillation apparatus, a mixture of 3,3-Dimethyl-indan-1-ol (63.2 g, 0.39 mole) and KHSO₄ (5.3 g, 0.039 mole) were heated slowly to an oven temperature of 95-100° C., under vacuum of Teflon vacuum pump (receiving flask being cooled in a dry ice/acetone bath). Dehydration began at ˜70° C. When dehydration was complete, the distillate was warmed to room temperature, diluted with dichloromethane and water and were separated. The organic solution was dried (NaSO₄), and the solvent evaporated slowly at ˜40° C. followed by an additional hr at ˜50° C. to give 1,1-Dimethyl-1H-indene (53.2 g) as a slightly pale yellow liquid. ¹H NMR (300 MHz, CDCl₃) [symbol] 7.25-7.35 (m, 2H) 7.15-7.25 (m, 2H) 6.6 (d, 1H) 6.35 (d, 1H) 1.30 (s, 6H).

### Preparation 29

### 1,1-DIMETHYL-INDAN-2-ONE

In a 5 L flask was charged 88% formic acid (385 ml) and 35% hydrogen peroxide (66 ml, 0.753 mole, 1.37 eq) and the mixture heated to 50° C. 1,1-Dimethyl-1H-indene (79.2 g, 0.55 mole) was added over 35 min while maintaining the temperature at 50-53° C. When addition was complete, the red-orange reaction solution was heated at 55° C. for 7 h then allowed to stand over a weekend. Solid NaHSO₃ (21.1 g, 0.202 mole, 0.37 eq) was added, and the reaction solution concentrated to ˜⅓ volume. The concentrated reaction mixture was added to a hot aq H₂SO₄ (3 L of 7% by volume), and the mixture steam distilled until 2.25 L of distillate was collected. The distillate was extracted with dichloromethane, and the organic solution washed with sat'd NaHCO₃, brine, dried (Na₂SO₄) and solvent evaporated to give 1,1-Dimethyl-indan-2-one (72.1 g) as an orange liquid. This material was distilled through a short path distillation head to give 71.6 g of 1,1-Dimethyl-indan-2-one as a pale yellowish-orange liquid. 1H NMR (400 MHz, CHLOROFORM-D) δ ppm 1.31 (s, 6 H) 3.58 (s, 2 H) 7.24 (m, 2 H) 7.29 (m, 2 H).

### Preparation 30

### 1,1-DIMETHYL-INDAN-2-ONE OXIME

A solution of 1,1-Dimethyl-indan-2-one (3.00 g, 18.7 mmol), hydroxylamine hydrochloride (2.60 g, 37.5 mmol) in pyridine (50 mL) was heated to 80° C. for 6 h, then allowed to cool. The solvent was removed in vacuo, and the residue was partitioned between CH₂Cl₂ (200 mL) and water (50 mL). The organic layer was separated, washed with water, brine, dried over Na₂SO₄, filtered, and concentrated in vacuo to provide a yellow oil that was co-evaporated with toluene then chloroform to provide 1,1-Dimethyl-indan-2-one oxime (3.20 g, 98%) as a yellow solid: ¹H NMR (300 MHz, CDCl₃) [symbol] 8.60 (br s, 1H), 7.28-7.21 (m, 4H), 3.88 (s, 2H), 1.47 (s, 6H); ESI MS m/z 176 [C₁₁H₁₃NO+H]⁺.

### Preparation 31

### N-(1,1-DIMETHYL-INDAN-2-YL)-ACETAMIDE

To a solution of 1,1-Dimethyl-indan-2-one oxime (10.0 g, 57.0 mmol) in ethanol (120 mL) was added Raney Ni (11.0 g, 50% in H₂O) which had been previously washed with ethanol (4×15 mL). The mixture was then hydrogenated at 40 psi for overnight. The reaction mixture was filtered via a pad of Celite®, and the filtrate was concentrated. The residue was dissolved in CH₂Cl₂ (220 mL), and the solution was dried over Na₂SO₄, concentrated to give the amine (8.00 g, 87%, crude) as a brown liquid. To a solution of the above amine (8.00 g, 49.7 mmol) in methylene chloride (150 mL) was added acetic anhydride (12.0 mL, 127 mmol) and triethylamine (30.0 mL, 216 mmol), and the mixture was stirred at rt for 3 h. The reaction mixture was diluted with CH₂Cl₂ (150 mL), washed with water, brine, dried over Na₂SO₄, and evaporated. The residue was purified by chromatography (silica gel, gradient from 50 to 60% EtOAc/hexanes) to provide N-(1,1-Dimethyl-indan-2-yl)-acetamide as a brown oil (5.50 g, 55%): ¹H NMR (300 MHz, CDCl₃) [symbol] 7.25-7.13 (m, 4H), 5.55 (d, J=7.6 Hz, 1H), 4.52 (m, 1H), 3.29 (dd, J=7.2, 7.0 Hz, 1H), 2.67 (dd, J=7.0, 7.0 Hz, 1H), 2.02 (s, 3H), 1.31 (s, 3H), 1.14 (s, 3H); ESI MS m/z 204 [C₁₃H₁₇NO+H]⁺.

### Preparation 32

### N-[5-(2-CHLOROACETYL)-1,1-DIMETHYL-INDAN-2-YL]-ACETAMIDE AND N-[6-(2-CHLOROACETYL)-1,1-DIMETHYL-INDAN-2-YL]-ACETAMIDE

Into a 250-mL round-bottom flask under N₂ was placed N-(1,1-Dimethyl-indan-2-yl)-acetamide (5.30 g, 26.0 mmol), aluminum chloride (20.8 g, 156 mmol), and methylene chloride (80 mL). Chloroacetyl chloride (3.31 mL, 41.6 mmol) was added dropwise and the reaction was heated to 40° C. for 5 h. After cooling down, the reaction mixture was poured into ice-water (300 mL) while stirring. The mixture was extracted with methylene chloride (2×200 mL). The combined organic extracts were washed with saturated NaHCO₃, water, brine, dried over Na₂SO₄, and evaporated. The residue was purified by chromatography (silica gel, gradient from 2 to 3% MeOH/CH₂Cl₂) to give a mixture of inseparable regioisomers N-[5-(2-Chloroacetyl)-1,1-dimethyl-indan-2-yl]-acetamide and N-[6-(2-Chloroacetyl)-1,1-Dimethyl-indan-2-yl]-acetamide (5.15 g, 71%) as a pale-yellow solid: ¹H NMR (300 MHz, CDCl₃) [symbol] 7.84-7.75 (m, 2H), 7.32-7.24 (m, 1H), 5.56 (d, J=7.0 Hz, 1H), 4.69 (s, 1H), 4.67 (s, 1H), 4.63-4.55 (m, 1H), 3.37-3.29 (m, 1H), 2.75 (d, J=7.6 Hz, 0.5H), 2.70 (d, J=7.6 Hz, 0.5H), 2.04 (s, 3H), 1.36 (s, 1.5 H), 1.35 (s, 1.5 H), 1.17(s, 1.5 H), 1.16 (s, 1.5 H); ESI MS m/z 280 [C₁₅H₁₈ClNO₂+H]⁺; HPLC t_{R}=15.3 min, t_{R′}=15.5 min.

### Preparation 33

### N-[5-(2-CHLOROETHYL)-1,1-DIMETHYL-INDAN-2-YL]-ACETAMIDE AND N-[6-(2-CHLOROETHYL)-1,1-DIMETHYL-INDAN-2-YL]-ACETAMIDE

Into a 100-mL sealed tube was placed the mixture of N-[5-(2-Chloroacetyl)-1,1-dimethyl-indan-2-yl]-acetamide and N-[6-(2-Chloroacetyl)-1,1-Dimethyl-indan-2-yl]-acetamide (3.90 g, 14.0 mmol), boron trifluoride diethyl etherate (10.6 mL, 83.8 mmol), and triethylsilane (13.5 mL, 83.8 mmol). The resulting mixture was placed in a pre-heated oil-bath at 80° C. for 2 h. After cooling down, the reaction was quenched by the addition of ice-water (50 mL) and the mixture was extracted with CH₂Cl₂ (2×200 mL). The combined extracts were washed with saturated NaHCO₃, water, brine, dried over Na₂SO₄, and evaporated. The residue was purified by chromatography (silica gel, 2.5% MeOH/CH₂Cl₂) and dried in a vacuum oven at 40° C. to give a mixture of inseparable regioisomers N-[5-(2-Chloroethyl)-1,1-dimethyl-indan-2-yl]-acetamide and N-[6-(2-Chloroethyl)-1,1-dimethyl-indan-2-yl]-acetamide. (3.30 g, 89%) as a thick oil that was solidified upon standing: ¹H NMR (300 MHz, CDCl₃) [symbol] 7.15-6.99 (m, 3H), 5.56 (d, J=8.8 Hz, 1H), 4.57-4.48 (m, 1H), 3.73-3.68 (m, 2H), 3.30-3.22 (m,1H), 3.08-3.01 (m, 2H), 2.68-2.59 (m, 1H), 2.02 (s, 3H), 1.304 (s, 1.5 H) 1.300 (s, 1.5 H), 1.14(s, 1.5 H), 1.13 (s, 1.5 H); ESI MS m/z 266 [C₁₅H₂₀ClNO+H]⁺.

### Examples 115-116

### N-{5-[2-(4-BENZO[D]ISOTHIAZOL-3-YL-PIPERAZIN-1-YL)-ETHYL]-1,1-DIMETHYL-INDAN-2-YL}-ACETAMIDE AND N-{6-[2-(4-BENZO[D]ISOTHIAZOL-3-YL-PIPERAZIN-1-YL)-ETHYL]-1,1-DIMETHYL-INDAN-2-YL}-ACETAMIDE

A mixture of compounds N-[5-(2-Chloroethyl)-1,1-dimethyl-indan-2-yl]-acetamide and N-[6-(2-Chloroethyl)-1,1-dimethyl-indan-2-yl]-acetamide (1.63 g, 6.15 mmol), 3-piperazin-1-yl-benzo[d]isothiazole hydrochloride (1.96 g, 7.69 mmol), potassium carbonate (2.55 g, 18.4 mmol), sodium iodide (1.02 g, 6.76 mmol) in acetonitrile (120 mL) was stirred at reflux for 60 h. After removal of solvent, the residue was partitioned in CH₂Cl₂/H₂O (200 mL/50 mL). The organic layer was separated and the water layer was extracted with CH₂Cl₂ (60 mL). The combined organic extracts were washed with water, brine, dried over Na₂SO₄, and evaporated. The residue was purified by chromatography (silica gel, gradient from 1 to 2% MeOH/EtOAc) to provide two regioisomers N-{5-[2-(4-Benzo[d]isothiazol-3-yl-piperazin-1-yl)-ethyl]-1,1-dimethyl-indan-2-yl}-acetamide (655 mg) and N-{6-[2-(4-Benzo[d]isothiazol-3-yl-piperazin-1-yl)-ethyl]-1,1-dimethyl-indan-2-yl}-acetamide (440 mg).

### Example 115

### N-{5-[2-(4-BENZO[D]ISOTHIAZOL-3-YL-PIPERAZIN-1-YL)-ETHYL]-1,1-DIMETHYL-INDAN-2-YL}-ACETAMIDE

(655 mg, 24%) as a pale yellow solid: mp 58-68° C.; ¹H NMR (300 MHz, CDCl₃) [symbol] 7.92(d, J=8.1 Hz, 1H), 7.82 (d, J=8.1 Hz, 1H), 7.47 (td, J=7.0, 0.9 Hz, 1H), 7.36 (td, J=8.0, 1.0 Hz, 1H), 7.10-7.05 (m, 3H), 5.53 (d, J=9.5 Hz, 1H), 4.57-4.49 (m, 1H), 3.61 (t, J=4.8 Hz, 4H), 3.27 (dd, J=7.3, 7.2 Hz, 1H), 2.87-2.61 (m, 9H), 2.02 (s, 3H), 1.30 (s, 3H), 1.14 (s, 3H); ESI MS m/z 449 [C₂₆H₃₂N₄OS+H]⁺; R_{f} 0.25 (40:1 CH₂Cl₂/MeOH); HPLC 98.1% (AUC), t_{R}=12.87 min. Anal. Calc'd for C₂₆H₃₂N₄OS.0.5H₂O: C, 68.24; H, 7.27; N, 12.24. Found: C, 68.12; H, 7.37; N, 12.13.

### Example 116

### N-{6-[2-(4-BENZO[D]ISOTHIAZOL-3-YL-PIPERAZIN-1-YL)-ETHYL]-1,1-DIMETHYL-INDAN-2-YL}-ACETAMIDE

(440 mg, 16%) as a pale yellow solid: mp 62-72° C.; ¹H NMR (300 MHz, CDCl₃) [symbol] 7.92(d, J=8.1 Hz, 1H), 7.82 (d, J=8.1 Hz,1H), 7.47 (td, J=7.0, 1.0 Hz, 1H), 7.36 (td, J=8.0, 1.0 Hz, 1H), 7.13 (d, J=7.6 Hz, 1H), 7.05 (d, J=7.6 Hz, 1H), 7.01 (s, 1H), 5.53 (d, J=9.5 Hz, 1H), 4.57-4.47 (m, 1H), 3.61 (t, J=4.8 Hz, 4H), 3.26 (dd, J=7.2, 7.2 Hz, 1H), 2.89-2.59 (m, 9H), 2.02 (s, 3H), 1.31 (s, 3H), 1.15 (s, 3H); ESI MS m/z 449 [C₂₆H₃₂N₄OS+H]⁺; R_{f} 0.29 (40:1 CH₂Cl₂/MeOH); HPLC 96.1% (AUC), t_{R}=12.63 min. Anal. Calc'd for C₂₆H₃₂N₄OS.0.5H₂O: C, 68.24; H, 7.27; N, 12.24. Found: C, 68.11; H, 7.50; N, 11.96.

### Examples 115-116A-C

### N-{5-[2-(4-BENZO[D]ISOTHIAZOL-3-YL-PIPERAZIN-1-YL)-ETHYL]-1,1-DIMETHYL-INDAN-2-YL}-ACETAMIDE AND N-{6-[2-(4-BENZO[D]ISOTHIAZOL-3-YL-PIPERAZIN-1-YL)-ETHYL]-1,1-DIMETHYL-INDAN-2-YL}-ACETAMIDE (RACEMATES AND (+) AND (−) ENANTIOMERS)

A suspension of piperazinylbenzisothiazole hydrochloride (16.50 g, 75.3 mmol), amidoindane as a mixture of regioisomers (10.00 g, 37.63 mmol) and Na₂CO₃ (5.98 g, 56.4 mmol) in water (180 ml) split evenly into 6 microwave reactor vessels, were heated to 175° C. for 30 min under microwave assistance. Upon cooling, MS indicated only desired product with no starting chloroethyl amidoindane. The reactions were diluted with ethyl acetate and water then combined. The layers were separated and the organics washed with water (100 ml), dried (MgSO4) and concentrated to a viscous residue. The regioisomers were separated by chomatography (30% ethyl acetate/Hex) and identified by NMR 2D-NOE, then each stereoisomer was separated using chiral HPLC from a portion of each racemate and finally isolated as its HCl salt. Total reaction conversion was 95% based on recovered desired products.

### Example 115A

### N-{5-[2-(4-BENZO[D]ISOTHIAZOL-3-YL-PIPERAZIN-1-YL)-ETHYL]-1,1-DIMETHYL-INDAN-2-YL}-ACETAMIDE

1H NMR (400 MHz, DMSO-D6) δ ppm 0.94 (s, 3 H) 1.16 (s, 3 H) 1.84 (s, 3 H) 2.56 (m, 2 H) 2.68 (q, J=13.35 Hz, 7 H) 2.95 (dd, J15.63, 7.62 Hz, 1 H) 3.43 (s, 4 H) 4.24 (q, J=8.60 Hz, 1 H) 7.02 (s, 3 H) 7.40 (t, J=7.62 Hz, 1 H) 7.52 (t, J=7.52 Hz, 1 H) 7.95 (d, J=8.99 Hz, 1 H) 8.01 (dd, J=8.21, 3.52 Hz, 2 H) chiral HPLC: two enantiomers, retention time (r.t.) 9.78 and 19.73 min., ChiralCel OJ (250×4.6 mm), 80:20 Hexane/EtOH; LCMS: Phenomenex Develosil Combi RP3 50×4.6 mm column, 45° C., 90-2% H₂O/MeCN w/0.1% HCO₂H over 3.5 min, hold 0.5 min, total run time 4.0 min. Results: 100% purity @254 nM, M+=449, r.t.=2.68 min.

### Example 115B

### (+)-N-{5-2-(4-BENZO[D]ISOTHIAZOL-3-YL-PIPERAZIN-1-YL)-ETHYL]-1,1-DIMETHYL-INDAN-2-YL}-ACETAMIDE

1H NMR (400 MHz, DMSO-D6) δ ppm 0.94 (s, 3 H) 1.16 (s, 3 H) 1.82 (s, 3 H) 2.69 (dd, J=15.75, 8.91 Hz, 1 H) 3.00 (m, J=12.74, 12.74, 8.70, 6.23, 6.23 Hz, 3 H) 3.30 (m, 4 H) 3.49 (t, J=11.96 Hz, 3 H) 3.62 (d, J=11.23 Hz, 2 H) 4.04 (d, J=13.43 Hz, 2 H) 4.25 (q, J=8.79 Hz, 1 H) 7.07 (m, J=7.93, 5.55, 4.36, 4.36 Hz, 3 H) 7.42 (t, J=7.32 Hz, 1 H) 7.55 (t, J=7.32 Hz, 1 H) 7.88 (d, J=8.79 Hz, 1 H) 8.06 (d, J=8.06 Hz, 1 H) 8.10 (d, J=8.06 Hz, 1 H) 11.37 (s, 1 H) chiral HPLC: 100% purity, r.t. 9.66 min., ChiralCel OJ (250×4.6 mm), 80:20 Hexane/EtOH; LCMS: Phenomenex Develosil Combi RP3 50×4.6 mm column, 45° C., 90-2% H₂O/MeCN w/0.1% HCO₂H over 3.5 min, hold 0.5 min, total run time 4.0 min. Results: 100% purity @254 nM, M+=449, r.t.=2.69 min.; [a]25/589=+7.57.

### Example 115C

### (−)-N-{5-[2-(4-BENZO[D]ISOTHIAZOL-3-YL-PIPERAZIN-1-YL)-ETHYL]-1,1-DIMETHYL-INDAN-2-YL}-ACETAMIDE

1H NMR (400 MHz, DMSO-D6) δ ppm 0.94 (s, 3 H) 1.16 (s, 3 H) 1.82 (s, 3 H) 2.68 (dd, J=15.87, 9.03 Hz, 1 H) 2.99 (m, 3 H) 3.30 (m, 4 H) 3.47 (t, J=12.09 Hz, 2 H) 3.62 (d, J=11.47 Hz, 2 H) 4.05 (d, J=13.67 Hz, 2 H) 4.25 (q, J=8.79 Hz, 1 H) 7.07 (d, J=7.87 Hz, 1H) 7.09 (s, 1H, NOE with m, 2.99 ppm, 3H and with m, 3.30 ppm, 4H) 7.11 (d, J=7.11 Hz, 1H, NOE with s, 0.94 ppm, 3H and with s, 1.16 ppm, 3H) 7.42 (t, J=7.20 Hz, 1 H) 7.55 (t, J=7.20 Hz, 1 H) 7.88 (d, J=9.03 Hz, 1 H) 8.06 (d, J8.06 Hz, 1 H) 8.10 (d, J=8.30 Hz, 1 H) 11.22 (s, 1 H) chiral HPLC: 100% purity, r.t. 19.78 min, ChiralCel OJ (250×4.6 mm), 80:20 Hexane/EtOH; LCMS: Phenomenex Develosil Combi RP3 50×4.6 mm column, 45° C., 90-2% H₂O/MeCN w/0.1% HCO₂H over 3.5 min, hold 0.5 min, total run time 4.0 min. Results: 100% purity @254 nM, M+=449, r.t.=2.69 min.; [a]25/589=−9.04.

### Example 116A

### N-{6-[2-(4-BENZO[D]ISOTHIAZOL-3-YL-PIPERAZIN-1-YL)-ETHYL]-1,1-DIMETHYL-INDAN-2-YL}-ACETAMIDE

1H NMR (400 MHz, DMSO-D6) δ ppm 0.95 (s, 3 H) 1.17 (s, 3 H) 1.84 (s, 3 H) 2.56 (m, 2 H) 2.69 (m, 7 H) 2.94 (dd, J=15.43, 8.01 Hz, 1 H) 3.42 (s, 4 H) 4.25 (q, J=8.66 Hz, 1 H) 6.98 (d, J=7.46 Hz, 1 H) 7.00 (s, 1H, NOE with 1.17 ppm, s, 3H) 7.05 (d, J=7.43 Hz, 1 H, NOE with 2.69 ppm, m, 7H) 7.40 (t, J=7.72 Hz, 1 H) 7.52 (m, 1 H) 7.93 (d, J=9.18 Hz, 1 H) 8.01 (d, J=8.21 Hz, 2 H) chiral HPLC: two enantiomers r.t. 7.28 and 15.51 min., Chiralcel OJ (250×4.6 mm), 80:20 Hex/EtOH; LCMS: Phenomenex Develosil Combi RP3 50×4.6 mm column, 45° C., 90-2% H₂O/MeCN w/0.1% HCO₂H over 3.5 min, hold 0.5 min, total run time 4.0 min. Results: 100% purity @254 nM, M+=449, r.t.=2.64 min.

### Example 116B

### (+)-N-{6-[2-(4-BENZO[D]ISOTHIAZOL-3-YL-PIPERAZIN-1-YL)-ETHYL]-1,1-DIMETHYL-INDAN-2-YL}-ACETAMIDE

1H NMR (400 MHz, DMSO-D6) δ ppm 0.96 (s, 3 H) 1.18 (s, 3 H) 1.82 (s, 3 H) 2.66 (dd, J=15.75, 8.91 Hz, 1 H) 2.95 (dd, J=15.75, 7.69 Hz, 1 H) 3.03 (dd, J=10.50, 6.35 Hz, 2 H) 3.33 (m, 4H) 3.46 (t, J=13.06 Hz, 3H) 3.63 (d, J=11.72 Hz, 2 H) 4.06 (d, J=13.43 Hz, 2 H) 4.25 (dd, J=16.80, 8.80 Hz, 1 H) 7.02 (dd, J=11.84, 4.27 Hz, 2 H) 7.11 (d, J=7.57 Hz, 1H) 7.43 (t, J=7.20 Hz, 1 H) 7.55 (m, J=5.55, 5.55, 4.03, 0.98 Hz, 1 H) 7.87 (d, J=9.03 Hz, 1 H) 8.08 (dd, J=113.43, 8.06 Hz, 2 H) 11.05 (s, 1 H) chiral HPLC: 100% purity, r.t. 7.28 min., ChiralCel OJ (250×4.6 mm), 80:20 Hexane/EtOH; LCMS: Phenomenex Develosil Combi RP3 50×4.6 mm column, 45° C., 90-2% H₂O/MeCN w/0.1% HCO₂H over 3.5 min, hold 0.5 min, total run time 4.0 min. Results: 100% purity @254nM, M+=449, r.t.=2.69 min.; [a]25/589=+34.40

### Example 116C

### (−)-N-{6-[2-(4-BENZO[D]ISOTHIAZOL-3-YL-PIPERAZIN-1-YL)-ETHYL]-1,1-DIMETHYL-INDAN-2-YL}-ACETAMIDE

1H NMR (400 MHz, DMSO-D6) δ ppm 0.96 (s, 3 H) 1.18 (s, 3 H) 1.82 (s, 3 H) 2.66 (dd, J=15.87, 9.03 Hz, 1 H) 2.95 (dd, J=15.87, 7.57 Hz, 1 H) 3.03 (dd, J=10.74, 6.59 Hz, 2 H) 3.31 (m, J=18.71, 10.50, 9.74, 8.42 Hz, 4 H) 3.47 (t, J11.96 Hz, 2 H) 3.61 (s, 2 H) 4.05 (d, J13.67 Hz, 2 H) 4.25 (m, 1 H) 7.01 (dd, J=7.57, 1.46 Hz, 1 H) 7.04 (s, 1 H) 7.11 (d, J=7.57 Hz, 1 H) 7.43 (m, 1 H) 7.55 (t, J=7.20 Hz, 1 H) 7.87 (d, J=8.79 Hz, 1 H) 8.06 (d, J=8.30 Hz, 1 H) 8.09 (d, J=8.30 Hz, 1 H) 11.10 (s, 1 H) chiral HPLC: 100% purity, r.t. 14.62 min., ChiralCel OJ (250×4.6 mm), 80:20 Hexane/EtOH; LCMS: Phenomenex Develosil Combi RP3 50×4.6 mm column, 45° C., 90-2% H₂O/MeCN w/0.1% HCO₂H over 3.5 min, hold 0.5 min, total run time 4.0 min. Results: 100% purity @254 nM, M+=449, r.t.=2.65 min.; [a]25/589=−29.05.

### Example 117

### {5-[2-(4-BENZO[D]ISOTHIAZOL-3-YL-PIPERAZIN-1-YL)-ETHYL]-1,1-DIMETHYL-INDAN-2-YL}-ETHYLAMINE METHANESULFONATE

A solution of N-{5-[2-(4-Benzo[d]isothiazol-3-yl-piperazin-1-yl)-ethyl]-1,1-dimethyl-indan-2-yl}-acetamide (650 mg, 1.45 mmol) in THF (20 mL) was treated dropwise with a solution of BH₃ in THF (5.8 mL, 1.5 M, 8.7 mmol). The mixture was heated to reflux for 3 h, allowed to cool. The reaction was quenched with 1N HCl until gas evolution subsided. The mixture was heated to reflux for 1 h, allowed to cool. THF was then removed in vacuo. The aqueous residue was basified with 6 N NaOH, and extracted with CH₂Cl₂ (3×40 mL). The combined organic layers were dried over Na₂SO₄, filtered, and the solvent was removed in vacuo. The residue was purified by flash column chromatography (silica gel, 5% MeOH/CH₂Cl₂ with 0.5% Et₃N) to give the free base of {5-[2-(4-Benzo[d]isothiazol-3-yl-piperazin-1-yl)-ethyl]-1,1-dimethyl-indan-2-yl}-ethylamine (440 mg, 70%) as a colorless oil. {5-[2-(4-Benzo[d]isothiazol-3-yl-piperazin-1-yl)-ethyl]-1,1-dimethyl-indan-2-yl}-ethylamine (free base, 440 mg, 1.01 mmol) was dissolved in ethyl acetate (10 mL), and then treated with CH₃SO₃H (2M in Et₂O, 0.51 mL, 1.02 mmol). The reaction mixture was stirred at rt for 30 min. The precipitate was collected by filtration and dried in a vacuum oven at 60° C. overnight to give {5-[2-(4-Benzo[d]isothiazol-3-yl-piperazin-1-yl)-ethyl]-1,1-dimethyl-indan-2-yl}-ethylamine methanesulfonate (350 mg, 38%) as a white solid: mp 115-125° C. dec; ¹H NMR (300 MHz, CDCl₃) [symbol] 8.80 (br s, 1H), 7.89 (d, J=8.1 Hz, 1H), 7.83 (d, J=8.1 Hz, 1H), 7.49 (t, J=7.5 Hz, 1H), 7.38 (t, J=7.5 Hz, 1H), 7.15-7.06 (m, 3H), 3.88-3.68 (m, 4H), 3.49 (t, J=8.6 Hz, 1H), 3.39-2.96 (m, 12.7 H), 2.77 (s, 5.1H), 1.54-1.49 (m, 6H), 1.34 (s, 3H); ESI MS m/z 435 [C₂₆H₃₄N₄S+H]⁺; R_{f} 0.27 (10:1 CH₂Cl₂/MeOH); HPLC 97.9% (AUC), t_{R}=10.87 min. Anal. Calc'd for C₂₆H₃₄N₄S.1.7CH₃SO₃H.2H₂O: C, 52.47; H, 7.13; N, 8.84. Found: C, 52.74; H, 7.01; N, 8.74.

### Example 118

### N-{5-[2-(4BENZO[D]ISOTHIAZOL-3-YL-PIPERAZIN-1-YL)-ETHYL]-1,1-DIMETHYL-INDAN-2-YL}-N-ETHYL-ACETAMIDE METHANESULFONATE

To a solution of {5-[2-(4-Benzo[d]isothiazol-3-yl-piperazin-1-yl)-ethyl]-1,1-dimethyl-indan-2-yl}-ethylamine (free base, 140 mg, 0.323 mmol) in methylene chloride (10 mL) was added acetic anhydride (66 mg, 0.64 mmol) and triethylamine (131 mg, 1.29 mmol). The mixture was stirred at rt overnight. The reaction mixture was washed with water, brine, dried over Na₂SO₄, evaporated, and chromatographed (silica gel, 3% MeOH/CH₂Cl₂) to provide N-{5-[2-(4-Benzo[d]isothiazol-3-yl-piperazin-1-yl)-ethyl]-1,1-dimethyl-indan-2-yl}-N-ethyl-acetamide (free base, 142 mg, 92%) as a white foam. N-{5-[2-(4-Benzo[d]isothiazol-3-yl-piperazin-1-yl)-ethyl]-1,1-dimethyl-indan-2-yl}-N-ethyl-acetamide (free base, 142 mg, 0.300 mmol) was dissolved in ethyl acetate (6 mL), and then treated with CH₃SO₃H (2M in Et₂O, 0.15 mL, 0.30 mmol). The reaction mixture was stirred at rt for 30 min and evaporated under reduced pressure. The resulting solid was triturated with EtOAc, filtered, and dried in a vacuum oven at 35° C. to give N-{5-[2-(4-Benzo[d]isothiazol-3-yl-piperazin-1-yl)-ethyl]-1,1-dimethyl-indan-2-yl}-N-ethyl-acetamide methanesulfonate (130 mg, 57%) as a white solid: mp 60-70° C.; ¹H NMR (300 MHz, CDCl₃) [symbol] 11.00 (br s, 1H), 7.86 (d, J=8.7 Hz, 2H), 7.54 (t, J=7.5 Hz, 1H), 7.42 (t, J=7.6 Hz, 1H), 7.21-7.05 (m, 3H), 5.11 (dd, J=5.5, 4.9 Hz, 0.5H), 4.35-4.11 (m, 6.5H), 3.96 (t, J=12.6 Hz, 2H), 3.72 (d, J=11.3 Hz, 2H), 3.49-3.13 (m, 7H), 2.93 (s, 6H), 2.37 (s, 1.5 H), 2.33 (s, 1.5H), 1.28 (s, 1.5H), 1.27 (s, 1.5H), 1.24-1.11 (m, 6H); ESI MS m/z 477 [C₂₈H₃₆N₄OS+H]⁺; R_{f} 0.34 (100:3 CH₂Cl₂/MeOH); HPLC 97.0% (AUC), t_{R}=13.78 min. Anal. Calc'd for C₂₆H₃₄N₄S.2CH₃SO₃H.1.8H₂O: C, 51.38; H, 6.84; N, 7.99. Found: C, 51.60; H, 6.84; N, 7.64.

### Example 119

### {6-[2-(4-BENZO[D]ISOTHIAZOL-3-YL-PIPERAZIN-1-YL)-ETHYL]-1,1-DIMETHYL-INDAN-2-YL}-ETHYLAMINE METHANESULFONATE

A solution of N-{6-[2-(4-Benzo[d]isothiazol-3-yl-piperazin-1-yl)-ethyl]-1,1-dimethyl-indan-2-yl}-acetamide (630 mg, 1.40 mmol) in THF (15 mL) was treated dropwise with a solution of BH₃ in THF (5.5 mL, 1.5 M, 5.5 mmol). The mixture was heated to reflux for 3 h, allowed to cool. The reaction was quenched with 1N HCl until gas evolution subsided. The mixture was heated to reflux for 1 h, allowed to cool. THF was then removed in vacuo. The queous residue was basified with 6 N NaOH, and extracted with CH₂Cl₂ (2×50 mL). The combined organic layers were dried over Na₂SO₄, filtered, and the solvent was removed in vacuo. The residue was purified by flash column chromatography (silica gel, 5% MeOH/CH₂Cl₂ with 0.5% Et₃N) to give {6-[2-(4-Benzo[d]isothiazol-3-yl-piperazin-1-yl)-ethyl]-1,1-dimethyl-indan-2-yl}-ethylamine (free base, 438 mg, 72%) as a colorless oil. {6-[2-(4-Benzo[d]isothiazol-3-yl-piperazin-1-yl)-ethyl]-1,1-dimethyl-indan-2-yl}-ethylamine (free base, 438 mg, 1.01 mmol) was dissolved in ethyl acetate (10 mL), and then treated with CH₃SO₃H (2M in Et₂O, 1.0 mL, 2.0 mmol). The reaction mixture was stirred at rt for 30 min. The precipitate was collected by filtration and dried in a vacuum oven at 55° C. overnight to give {6-[2-(4-Benzo[d]isothiazol-3-yl-piperazin-1-yl)-ethyl]-1,1-dimethyl-indan-2-yl}-ethylamine methanesulfonate (305 mg, 38%) as a white solid: mp 125-135° C.; ¹H NMR (300 MHz, CDCl₃) [symbol] 8.80 (br s, 1H), 7.90 (d, J=8.1 Hz, 1H), 7.83 (d, J=8.1 Hz, 1H), 7.49 (td, J=7.5, 0.9 Hz, 1H), 7.38 (td, J=7.9, 0.9 Hz, 1H), 7.14-7.07 (m, 2H), 7.03 (s,1H), 3.84-3.64 (m, 4H), 3.49 (t, J=8.6 Hz, 1H), 3.39-2.90 (m, 12.4 H), 2.77 (s, 4.2H), 1.56-1.50 (m, 6H), 1.35 (s, 3H); ESI MS m/z 435 [C₂₆H₃₄N₄S+H]⁺; R_{f} 0.21 (20:1 CH₂Cl₂/MeOH); HPLC 98.4% (AUC), t_{R}=10.78 min. Anal. Calc'd for C₂₆H₃₄N₄S.1.4CH₃SO₃H.0.5H₂O: C, 56.92; H, 7.08; N, 9.69. Found: C, 56.60; H, 7.22; N, 9.42.

### Example 120

### N-{6-[2-(4-BENZO[D]ISOTHIAZOL-3-YL-PIPERAZIN-1-YL)-ETHYL]-1,1-DIMETHYL-INDAN-2-YL}-N-ETHYL-ACETAMIDE METHANESULFONATE

To a solution of {6-[2-(4-Benzo[d]isothiazol-3-yl-piperazin-1-yl)-ethyl]-1,1-dimethyl-indan-2-yl}-ethylamine (free base, 160 mg, 0.369 mmol) in methylene chloride (10 mL) was added acetic anhydride (75 mg, 0.74 mmol) and triethylamine (149 mg, 1.48 mmol). The mixture was stirred at rt overnight. The reaction mixture was washed with water, brine, dried over Na₂SO₄, evaporated, and chromatographed (silica gel, 3% MeOH/CH₂Cl₂) to provide N-{6-[2-(4-Benzo[d]isothiazol-3-yl-piperazin-1-yl)-ethyl-1,1-dimethyl-indan-2-yl}-N-ethyl-acetamide (free base, 158 mg, 92%) as a white foam. N-{6-[2-(4-Benzo[d]isothiazol-3-yl-piperazin-1-yl)-ethyl]-1,1-dimethyl-indan-2-yl}-N-ethyl-acetamide (free base, 158 mg, 0.330 mmol) was dissolved in ethyl acetate (8 mL), and then treated with CH₃SO₃H (2M in Et₂O, 0.17 mL, 0.34 mmol). The reaction mixture was stirred at rt for 30 min and evaporated under reduced pressure. The resulting solid was triturated with EtOAc, filtered, and dried in a vacuum oven at 35° C. overnight to give N-{6-[2-(4-Benzo[d]isothiazol-3-yl-piperazin-1-yl)-ethyl]-1,1-dimethyl-indan-2-yl}-N-ethyl-acetamide methanesulfonate (140 mg, 61%) as a white solid: mp 62-72° C.; ¹H NMR (300 MHz, CDCl₃) [symbol] 11.08 (br s, 1H), 7.86 (d, J=8.7 Hz, 2H), 7.54 (t, J=7.7 Hz, 1H), 7.42 (t, J=7.7 Hz, 1H), 7.22-7.05 (m, 3H), 5.13 (m, 0.5H), 5.00-4.55 (m, 4H), 4.33 (m, 0.5H), 4.18 (d, J=4.7 Hz, 2H), 3.97 (t, J=12.5 Hz, 2H), 3.72 (d, J=11.8 Hz, 2H), 3.45-3.18 (m, 7H), 2.93 (s, 6H), 2.36 (s, 1.5 H), 2.32 (s, 1.5H), 1.27 (s, 1.5H), 1.26 (s, 1.5H), 1.20-1.12 (m, 6H); ESI MS m/z 477 [C₂₈H₃₆N₄OS+H]⁺; R_{f} 0.34 (100:3 CH₂Cl₂/MeOH); HPLC >99% (AUC), t_{R}=13.75 min. Anal. Calc'd for C₂₆H₃₄N₄S.2CH₃SO₃H.1.5H₂O: C, 51.78; H, 6.81; N, 8.05. Found: C, 51.79; H, 6.89; N, 7.86.

### Preparation 34

### (3′,4′-DIHYDRO-1′H-SPIRO[[1,3]DIOXOLANE-2,2′-NAPHTHALEN]-6′-YL)-ACETIC ACID, ETHYL ESTER

A mixture of (6-oxo-5,6,7,8-tetrahydronaphthalen-2-yl)-acetic acid, ethyl ester (500 mg (2.15 mmol). Prepared according to EP-430,459 A1 ethylene glycol (1.20 ml, 21.5 mmol), and a catalytic amount of p-TsOH in benzene (60 ml) was heated at reflux using a Dean-Stark trap for removal of water. After 4 hours, reflux was stopped and the reaction mixture was diluted with EtOAc. The organic layer was washed with saturated, aqueous sodium bicarbonate solution, dried over magnesium sulfate, filtered, and concentrated to a yellow oil (594 mg, 100%) which was used without further purification. MS (APCI): (M+1)+=277. ¹H-NMR (CDCl₃, δ): 7.03 (m, 3H), 4.11 (q, 2H, J=7.1, 7.3, 7.1 Hz), 4.00 (m, 4H), 3.52 (s, 2H), 2.94 (m, 4H), 1.93 (t, 2H, J=6.6, 6.8 Hz), 1.23 (t, 3H, J=7.1, 7.3 Hz).

### Preparation 35

### 2-(3′,4′-DIHYDRO-1′H-SPIRO[[1,3]DIOXOLANE-2,2′-NAPHTHALEN]-6′-YL)ETHANOL

To a solution of the title compound from preparation 34 (594 mg, 2.15 mmol) in anhydrous THF (5 ml) under nitrogen, cooled to 0° C., is added dropwise 6.45 ml (6.45 mmol) of a 1.0 M diiso-butylaluminum hydride solution in THF (effervescence). After addition was complete, the reaction mixture was stirred at 0° C. for 1 hour and quenched by slow addition of MeOH. The mixture was partitioned between saturated, aqueous potassium sodium tartrate solution and EtOAc and the whole was stirred for 30 minutes. The aqueous layer was separated and washed with EtOAc and the organic layers were combined, dried (MgSO₄), filtered and concentrated to an oil, 471 mg (93%), which was used without further purification. MS (APCI): (M+1)⁺=235. ¹H-NMR (CDCl₃, δ): 6.98 (s, 3H), 4.01 (s, 4H), 3.81 (t, 2H, J=6.4, 6.6 Hz), 2.94 (m, 4H), 2.79 (t, 2H, J=6.6, 6.6 Hz), 1.93 (t, 2H, J=6.8, 6.8 Hz).

### Preparation 36

### TOLUENE-4-SULFONIC ACID-2-(3′,4′-DIHYDRO-1′H-SPIRO[[1,3]DIOXOLANE-2,2′-NAPHTHALEN]-6′-YL)-ETHYL ESTER

To a mixture of the title compound from Preparation 35 (471 mg, 2.01 mmol) and p-toluenesulfonyl chloride (421 mg, 2.21 mmol, recrystallized from hexanes) in anhydrous THF (10 ml) under nitrogen was added dropwise triethylamine (0.314 ml, 2.21 mmol, dried over NaOH). After addition was complete, the reaction mixture was stirred at ambient temperature for 40 hours and partitioned between water and EtOAc. The organic layer was dried (MgSO₄), filtered, and concentrated. The crude product was purified by elution through a flash column (silica gel 60, 230-400 mesh, 7:3 hexanes:EtOAc) to give a clear oil, 531 mg (68%). MS (APCI): (M+1)⁺=389. ¹H-NMR (CDCl₃, δ): 7.65 (d, 2H, J=8.3 Hz), 7.26 (d, 2H, J=7.8 Hz), 6.85 (m, 3H), 4.13 (t, 2H, J=7.1, 7.3 Hz), 4.01 (s, 4H), 2.90 (m, 6H), 2.41 (s, 3H), 1.91 (t, 2H, J=6.8, 6.8 Hz).

### Example 121

### 3-{4-[2-(3′,4′-DIHYDRO-1′H-SPIRO[[1,3]DIOXOLANE-2,2′-NAPHTHALEN]-6′-YL)-ETHYL]-PIPERAZIN-1-YL}-BENZO[D]ISOTHIAZOLE

A mixture of 3-(piperazin-1-yl)benzo[d]isothiazole hydrochloride (349 mg, 1.36 mmol) and the title compound from Preparation 36 (530 mg, 1.36 mmol) with anhydrous K₂CO₃ (376 mg, 2.72 mmol) in acetonitrile (20 ml) was heated at reflux for 24 hours. The reaction mixture was filtered and the filtrate was concentrated. The crude product was purified by elution through a flash column (silica gel 60, 230-400 mesh, 1:1 hexanes:EtOAc) to give a clear oil which crystallized on standing, 497 mg (84%). MS (APCI): (M+1)⁺=436. ¹H-NMR (CDCl₃, δ): 7.89 (d, 1H, J=7.8 Hz), 7.80 (d, 1H, J=8.1 Hz), 7.46 (t, 1H, J=7.3, 7.3 Hz), 7.34 (t, 1H, J=7.3, 7.3 Hz), 6.98 (s, 3H), 4.01 (s, 4H), 3.58 (br s, 4H), 2.95-2.65 (m, 12H), 1.93 (t, 2H, J=6.8, 6.8 Hz). CHN, calc'd for C₂₅H₂₉N₃O₂S: C, 68.94%; H, 6.71%; N, 9.65%. found: C, 68.75%; H, 6.70%; N, 9.54%.

### Example 122

### 6-[2-(4-BENZO[D]ISOTHIAZOL-3-YL-PIPERAZIN-1-YL)-ETHYL]-3,4-DIHYDRO-1H-NAPHTHALEN-2-ONE HYDROCHLORIDE

A solution of the title compound Example 121 (451 mg, 1.04 mmol) in acetone (20 ml) with aqueous 1.0 N HCl (2.0 ml) was heated at reflux for 2 hours (after the first 5 minutes of reflux, a white solid precipitated). Upon cooling to room temperature the precipitated product was collected and washed with acetone to give 429 mg (96%). MS (APCI): (M+1)⁺=392, (M−1)⁺=390. ¹H-NMR (DMSO-d₆, δ): 8.08 (m, 2H), 7.57 (t, 1H, J=7.3, 7.6 Hz), 7.44 (t, 1H, J=7.8, 7.3 Hz), 7.18 (s, 1H), 7.11 (s, 2H), 4.07 (br d, 2H, J=13.7 Hz), 3.65 (br d, 2H, J=12.2 Hz), 3.54 (s, 2H), 3.47-3.35 (m, 6H), 3.04 (m, 2H), 2.98 (t, 2H, J=6.6, 6.6 Hz), 2.40 (t, 2H, J=6.6, 6.6 Hz). CHN, calc'd for C₂₃H₂₅N₃OS HCl: C, 64.55%; H, 6.12%; N, 9.82%. found: C, 64.61%; H, 6.26%; N, 9.76%.

### Example 123

### 6-[2-(4-BENZO[D]ISOTHIAZOL-3-YL-PIPERAZIN-1-YL)-ETHYL]-1,2,3,4-TETRAHYDRONAPHTHALEN-2-YLAMINE

A mixture of the title compound from Example 122 (300 mg, 0.70 mmol) with ammonium acetate (54 mg, 0.70 mmol) and sodium cyanoborohydride (102 mg, 1.6 mmol) in anhydrous MeOH (15 ml) was stirred at ambient temperature under nitrogen for 48 hours. The reaction mixture was diluted with aqueous NaHCO₃ solution and extracted with dichloromethane. The organic extract was dried (MgSO₄), filtered, and concentrated to a white, foamy solid, 222 mg (81%). MS (APCI): (M+1)⁺=393, (M+2)⁺=394. ¹H-NMR (CDCl₃, δ): 7.89 (d,1H, J=8.3 Hz), 7.80 (d, 1H, J=8.3 Hz), 7.45 (t, 1H, J=7.1, 7.1 Hz), 7.34 (t, 1H, J=7.3, 7.1 Hz), 6.99 (m, 3H), 3.60 (m, 4H), 3.32-2.41 (m, 15H), 2.15 (m, 1H), 1.61 (m, 1H).

### Example 124

### {6-[2-(4-BENZO[D]ISOTHIAZOL-3-YL-PIPERAZIN-1-YL)-ETHYL]-1,2,3,4-TETRAHYDORNAPHTHALEN-2-YL}-METHYLAMINE

A mixture of the title compound from Example 122 (300 mg, 0.70 mmol) with methylamine hydrochloride (319 mg, 4.12 mmol) and sodium cyanoborohydride (51 mg, 7.0 mmol) in MeOH (10 ml) and the reaction mixture was stirred at ambient temperature for 4 days. The reaction mixture was diluted with water and extracted with dichloromethane. The organic extract was dried (MgSO₄), filtered, and concentrated to an off-white, foamy solid, 244 mg (86%). MS (APCI): (M+1)⁺=407. ¹H-NMR (CDCl₃, δ): 7.90 (d, 1H, J=8.3 Hz), 7.80 (d, 1H, J=8.3 Hz), 7.46 (t, 1H, J=7.1, 7.1 Hz), 7.36 (t, 1H, J=7.1, 7.1 Hz), 7.03 (s, 2H), 6.98 (s, 1H), 3.62 (br s, 4H), 3.33-3.17 (m, 2H), 3.00-2.70 (m, 15H), 2.16 (br s, 1H), 1.95 (m, 1H).

### Example 125

### N-{6-[2-(4-BENZO[D]ISOTHIAZOL-3-YL-PIPERAZIN-1-YL)-ETHYL]-1,2,3,4-TETRAHYDRONAPHTHALEN-2-YL}-N-METHYLACETAMIDE

To a solution of the title compound from Example 124 (242 mg, 0.60 mmol) and triethylamine (125 μl, 0.90 mmol, dried over NaOH) in anhydrous THF (5 ml) under nitrogen at room temperature was added dropwise acetyl chloride (64 μl, 0.90 mmol) with vigorous stirring. After 1 hour, the reaction mixture was partitioned between aqueous NaHCO₃ solution and dichloromethane. The organic layer was dried (MgSO₄) filtered, and concentrated and the crude product was eluted through a flash column (silica gel 60, 230-400 mesh, 3% MeOH in EtOAc to 5% MeOH in EtOAc) to give the title compound as a clear, glasslike solid, 140 mg (52%). MS (APCI): (M+1)⁺=449. ¹H-NMR (CDCl₃, δ): 7.90 (d, 1H, J=8.1 Hz), 7.80 (d, 1H, J=8.1 Hz), 7.45 (t, 1H, J=7.1, 8.3 Hz), 7.34 (t, 1H, J=7.8, 7.3 Hz), 6.97 (m, 3H), 4.88 (m, 1H), 3.99 (m, 1H), 3.58 (br s, 4H), 2.97-2.64 (m, 15H), 2.12 (d, 3H, J=6.6 Hz), 1.97 (m, 1H), 1.86 (m, 1H). CHN, calc'd for C₂₆H₃₂N₄OS (0.4 mol water): C, 68.51%; H, 7.25%; N, 12.29%. found: C, 68.22%; H, 7.30%; N, 12.31%.

The enantiomers of the title compound were isolated by elution of the racemate through a Chiralcel AD column (250×4.6 mm) with 40:60 hexanes (0.2% TFA):EtOH as eluent, flow rate=0.80 m/min, run time ˜42 minutes. The (−)-enantiomer had a retention time of 6.17 minutes with an optical rotation of −28° (589 nm, c=5, MeOH) and the (+)-enantiomer had a retention time of 6.77 minutes with an optical rotation of +32.80 (589 nm, c=5, MeOH).

### Example 126

### N-{6-[2-(4BENZO[D]ISOTHIAZOL-3-YL-PIPERAZIN-1-YL)-ETHYL]-1,2,3,4-TETRAHYDRO-NAPHTHALEN-2-YL}-ACETAMIDE

To a solution of the title compound of Example 123 (220 mg, 0.56 mmol) and triethylamine (117 μl, 0.84 mmol, dried over NaOH) in anhydrous THF (5 ml) at room temperature under nitrogen was added dropwise acetyl chloride (60 μl, 0.84 mmol) with vigorous stirring. After 1.5 hours, the reaction mixture was partitioned between sat'd NaHCO_{3(aq.)} and dichloromethane. The organic layer was dried (MgSO₄), filtered, concentrated, and the crude product was purified by elution through a flash column (silica gel 60, 230-400 mesh, 3% MeOH in EtOAc to 5% MeOH in EtOAc). The glasslike solid which was isolated was dissolved in dichloromethane and the solution was treated with 4.0 N HCl solution in dioxane to precipitate the HCl salt, 126 mg (48%). MS (APCI): (M+1)⁺=435, (M−1)⁺=433. ¹H-NMR (DMSO-d₆, δ): 8.10 (m, 2H), 7.57 (t, 1H, J=7.3, 7.1 Hz), 7.45 (t, 1H, J=7.3, 7.3 Hz), 7.01 (m, 3H), 4.08 (br d, 2H, J=12.5 Hz), 3.85 (m, 1H), 3.65 (br d, 2H, J=11.2 Hz), 3.32 (m, 6H), 2.88 (m, 5H), 2.47 (s, 3H), 2.06 (m, 2H), 1.79 (m, 2H). CHN, calc'd for C₂₅H₃₀N₄OS 1.4 HCl: C, 61.83%; H, 6.52%; N, 11.54%. found: C, 61.43%; H, 6.58%; N, 10.74%.

The enantiomers of the title compound were isolated by elution of the racemate through a Chiralpak AD column (250×4.6 mm) using 3:1 hexanes (0.19 DEA):EtOH as eluent, flow rate=0.50 ml/min. The (+)-enantiomer had a retention time of 20.1 minutes with an optical rotation of +46° (589 nm, c=1, CH₂Cl₂) and the (−)-enantiomer had a retention time of 22.8 minutes with an optical rotation of −36° (589 nm, c=1, CH₂Cl₂).

The methodology described in the preparation of Example 126 was appropriately applied to the preparation of Examples 127-130.

### Example 127

### N-{6-[2-(4-BENZO[D]ISOTHIAZOL-3-YL-PIPERAZIN-1-YL)-ETHYL]-1,2,3,4-TETRAHYDRO-NAPHTHALEN-2-YL}-PROPANAMIDE

From the title compound of Example 123 (281 mg, 0.72 mmol) and propionyl chloride (94 μl, 1.07 mmol). Isolated as the HCl salt, 155 mg (44%). MS (APCI): (M+1)⁺=449, (M−1)⁺=447. ¹H-NMR (DMSO-d₆, δ): 8.08 (m, 2H), 7.55 (t, 1H, J=7.6, 7.6 Hz), 7.42 (t, 1H, J=8.1, 7.1 Hz), 6.98 (m, 3H), 4.05 (br d, 2H, J=13.2 Hz), 3.63 (br d, 2H, J=11.5 Hz), 3.29 (m, 12H), 2.80 (m, 5H), 2.02 (m, 1H), 0.94 (m, 2H). CHN, calc'd for C₂₆H₃₂N₄OS HCl: C, 64.38%; H, 6.86%; N, 11.55%. found: C, 64.28%; H, 6.77%; N, 10.76%.

### Example 128

### N-{6-[2-(4-BENZO[D]ISOTHIAZOL-3-YL-PIPERAZIN-1-YL)-ETHYL]-1,2,3,4-TETRAHYDRO-NAPHTHALEN-2-YL}-BUTYRAMIDE

From the title compound of Example 123 (200 mg, 0.51 mmol) and butyryl chloride (80 μl, 0.77 mmol). Isolated as the HCl salt, 104 mg (41%). MS (APCI): (M+1)⁺=463, (M−1)⁺=461. ¹H-NMR (DMSO-d₆, δ): 8.10 (m, 2H), 7.57 (t, 1H, J=7.6, 7.8 Hz), 7.45 (t, 1H, J=7.8, 7.3 Hz), 7.00 (m, 3H), 4.08 (br d, 2H, J=12.0 Hz), 3.65 (br d, 2H, J=12.0 Hz), 3.31 (m, 10H), 2.85 (m, 6H), 2.02 (t, 2H, J=7.1, 7.3 Hz), 1.50 (q, 2H, J=7.3, 7.3, 7.3 Hz), 0.85 (quintet, 3H, J=7.3, 6.6, 7.1, 7.6 Hz). CHN, calc'd for C₂₇H₃₄N₄OS 1.1 HCl: C, 64.50%; H, 7.04%; N, 11.14%. found: C, 64.60%; H, 6.92%; N, 10.57%.

### Example 129

### N-{6-[2-(4BENZO[D]ISOTHIAZOL-3-YL-PIPERAZIN-1-YL)-ETHYL]-1,2,3,4-TETRAHYDRO-NAPHTHALEN-2-YL}-3-METHYL-BUTYRAMIDE (13)

From the title compound of Example 123 (200 mg, 0.51 mmol) and isovaleryl chloride (94 μl, 0.77 mmol). Isolated as the HCl salt, 107 mg (41%). MS (APCI): (M+1)⁺=477, (M−1)⁺=475. ¹H-NMR (DMSO-d₆, δ): 8.10 (m, 2H), 7.57 (t, 1H, J=7.3, 7.8 Hz), 7.44 (t, 1H, J=8.1, 7.3 Hz), 7.00 (m, 3H), 4.08 (br d, 2H, J=12.2 Hz), 3.65 (br d, 2H, J=11.2 Hz), 3.36 (m, 13H), 2.90 (m, 4H), 1.93 (m, 2H), 0.86 (m, 6H). CHN, calc'd for C₂₈H₃₆N₄OS 1.1 HCl: C, 65.08%; H, 7.24%; N, 10.84%. found: C, 64.68%; H, 7.06%; N, 10.35%.

### Example 130

### CYCLOPENTANECARBOXYLIC ACID-{6-[2-(4-BENZO[D]ISOTHIAZOL-3-YL-PIPERAZIN-1-YL)-ETHYL-1,2,3,4-TETRAHYDRONAPHTHALEN-2-YL}-AMIDE

From the title compound of Example 123 (200 mg, 0.51 mmol) and cyclopentanecarbonyl chloride (100 μl, 0.77 mmol). Isolated as the HCl salt, 37 mg (14%). MS (APCI): (M+1)⁺=489. ¹H-NMR (DMSO-d₆, δ): 8.08 (m, 2H), 7.55 (t, 1H, J=7.6, 7.6 Hz), 7.42 (t, 1H, J=7.8, 7.6 Hz), 6.98 (m, 3H), 4.06 (br d, 2H, J=12.7 Hz), 3.63 (br d, 2H, J=10.2 Hz), 3.29 (m, 9H), 2.82 (m, 4H), 2.44 (m, 8H), 1.60 (m, 4H). CHN, calc'd for C₂₉H₃₆N₄OS 1.2 HCl: C, 65.42%; H, 7.04%; N, 10.52%. found: C, 64.94%; H, 6.89%; N, 10.14%.

### Example 131

### 1-METHYL-CYCLOPROPANECARBOXYLIC ACID {6-[2-(4-BENZO[D]ISOTHIAZOL-3-YL-PIPERAZIN-1-YL)-ETHYL]-1,2,3,4-TETRAHYDRO-NAPHTHALEN-2-YL}AMIDE

6-[2-(4-Benzo[d]isothiazol-3-yl-piperazin-1-yl)-ethyl]-1,2,3,4-tetrahydro-naphthalen-2-ylamine was diluted to 0.10 M with anhydrous dichloromethane, then delivered to an 8 mL vial via pipette (0.10 mmol). 1-Methyl-cyclopropanecarbonyl chloride was diluted to 1.0 M with anhydrous dichloromethane then added to the amine solution (0.20 mmol). PS-Diisopropylethyl amine was added (0.20 mmol), and the reaction was shaken overnight at room temperature. The following morning the reaction was diluted with 1 mL of anhydrous dichloromethane. PS-Trisamine scavenger resin was then added (0.20 mmol). The reaction was stirred for 3 h at room temperature. The resin was filtered and washed with one mL dichloromethane. The filtrate was concentrated via HT-12 GeneVac. Crude product was purified by HPLC (30×100 mm ODS-A C(18) 5u column). 1-Methyl-cyclopropanecarboxylic acid {6-[2-(4-benzo[d]isothiazol-3-yl-piperazin-1-yl)-ethyl]-1,2,3,4-tetrahydro-naphthalen-2-yl}amide was recovered in 90% purity @ 214 nm, LCMS (APCI) 475 [M+H]⁺.

Examples 132-165 were synthesized in combinatorial library format following the steps outlined in Example 131 on a 0.10 mmol scale using 6-[2-(4-Benzo[d]isothiazol-3-yl-piperazin-1-yl)-ethyl]-1,2,3,4-tetrahydro-naphthalen-2-ylamine with appropriate acid chloride or isocyanate starting materials and PS-Diisopropylethylamine. The crude products were purified by HPLC (30×100 mm ODS-A C (18) 5u column).

| Example | | |
|---|---|---|
| Number | Compound Name | Data |
| 132 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 515[M+H]⁺ |
| | NAPHTHALEN-2-YL}-2-FLUORO- | |
| | BENZAMIDE | |
| 133 | FURAN-2-CARBOXYLIC ACID {6-[2- | Isolated in 90% purity @ |
| | (4-BENZO[D]ISOTHIAZOL-3-YL- | 214nm; LCMS(APCI) |
| | PIPERAZIN-1-YL)-ETHYL]-1,2,3,4- | 487[M+H]⁺ |
| | TETRAHYDRO-NAPHTHALEN-2- | |
| | YL}-AMIDE | |
| 134 | PENTANOIC ACID {6-[2-(4- | Isolated in 94% purity @ |
| | BENZO[D]ISOTHIAZOL-3-YL- | 214nm; LCMS(APCI) |
| | PIPERAZIN-1-YL)-ETHYL]-1,2,3,4- | 477[M+H]⁺ |
| | TETRAHYDRO-NAPHTHALEN-2- | |
| | YL}-AMIDE | |
| 135 | N-{6-[2-(4-BENZO[D]ISOTHIAZOL-3- | solated in 96% purity @ 214nm; |
| | YL-PIPERAZIN-1-YL)-ETHYL]- | LCMS(APCI) |
| | 1,2,3,4-TETRAHYDRO- | 515[M+H]⁺ |
| | NAPHTHALEN-2-YL}-4-FLUORO- | |
| | BENZAMIDE | |
| 136 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 497[M+H]⁺ |
| | NAPHTHALEN-2-YL}-BENZAMIDE | |
| 137 | N-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 503[M+H]⁺ |
| | NAPHTHALEN-2-YL}-2- | |
| | CYCLOPENTYL-ACETAMIDE | |
| 138 | EXAMPLE 138 | Isolated in 100% purity @ |
| | N-{6-[2-(4-BENZO[D]ISOTHIAZOL-3- | 214nm; LCMS(APCI) |
| | YL-PIPERAZIN-1-YL)-ETHYL]- | 515[M+H]⁺ |
| | 1,2,3,4-TETRAHYDRO- | |
| | NAPHTHALEN-2-YL}-3-FLUORO- | |
| | BENZAMIDE | |
| 139 | N-{6-[2-(4-BENZO[D]ISOTHIAZOL-3- | Isolated in 91% purity @ |
| | YL-PIPERAZIN-1-YL)-ETHYL]- | 214nm; LCMS(APCI) |
| | 1,2,3,4-TETRAHYDRO- | 465[M+H]⁺ |
| | NAPHTHALEN-2-YL}-2-METHOXY- | |
| | ACETAMIDE | |
| 140 | N-{6-[2-(4-BENZO[D]ISOTHIAZOL-3- | Isolated in 96% purity @ |
| | YL-PIPERAZIN-1-YL)-ETHYL]- | 214nm; LCMS(APCI) |
| | 1,2,3,4-TETRAHYDRO- | 511[M+H]⁺ |
| | NAPHTHALEN-2-YL}-4-METHYL- | |
| | BENZAMIDE | |
| 141 | N-{6-[2-(4-BENZO[D]ISOTHIAZOL-3- | Isolated in 98% purity @ |
| | YL-PIPERAZIN-1-YL)-ETHYL]- | 214nm; LCMS(APCI) |
| | 1,2,3,4-TETRAHYDRO- | 511[M+H]⁺ |
| | NAPHTHALEN-2-YL}-2-PHENYL- | |
| | ACETAMIDE | |
| 142 | 1-{6-[2-(4-BENZO[D]ISOTHIAZOL-3- | Isolated in 91% purity @ |
| | YL-PIPERAZIN-1-YL)-ETHYL]- | 214nm; LCMS(APCI) |
| | 1,2,3,4-TETRAHYDRO- | 478[M+H]⁺ |
| | NAPHTHALEN-2-YL}-3-PROPYL- | |
| | UREA | |
| 143 | 1-{6-[2-(4-BENZO[D]ISOTHIAZOL-3- | Isolated in 94% purity @ |
| | YL-PIPERAZIN-1-YL)-ETHYL]- | 214nm; LCMS(APCI) |
| | 1,2,3,4-TETRAHYDRO- | 492[M+H]⁺ |
| | NAPHTHALEN-2-YL}-3-BUTYL- | |
| | UREA | |
| 144 | 1-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 478[M+H]⁺ |
| | NAPHTHALEN-2-YL}-3- | |
| | ISOPROPYL-UREA | |
| 145 | 1-{6-[2-(4-BENZO[D]ISOTHIAZOL-3- | Isolated in 96% purity @ |
| | YL-PIPERAZIN-1-YL)-ETHYL]- | 214nm; LCMS(APCI) |
| | 1,2,3,4-TETRAHYDRO- | 526[M+H]⁺ |
| | NAPHTHALEN-2-YL}-3-BENZYL- | |
| | UREA | |
| 146 | CYCLOPROPANECARBOXYLIC | Isolated in 99% purity @ |
| | ACID {6-[2-(4- | 214nm; LCMS(APCI) |
| | BENZO[D]ISOTHIAZOL-3-YL- | 475[M+H]⁺ |
| | PIPERAZIN-1-YL)-ETHYL]-1,2,3,4- | |
| | TETRAHYDRO-NAPHTHALEN-2- | |
| | YL}-METHYL-AMIDE | |
| 147 | N-{6-[2-(4-BENZO[D]ISOTHIAZOL-3- | Isolated in 99% purity @ |
| | YL-PIPERAZIN-1-YL)-ETHYL]- | 214nm; LCMS(APCI) |
| | 1,2,3,4-TETRAHYDRO- | 463(M+H]⁺ |
| | NAPHTHALEN-2-YL}-N-METHYL- | |
| | PROPIONAMIDE | |
| 148 | 1-METHYL- | Isolated in 94% purity @ |
| | CYCLOPROPANECARBOXYLIC | 214nm, LCMS(APCI) |
| | ACID {6-[2-(4- | 489[M+H]⁺ |
| | BENZO[D]ISOTHIAZOL-3-YL- | |
| | PIPERAZIN-1-YL)-ETHYL]-1,2,3,4- | |
| | TETRAHYDRO-NAPHTHALEN-2- | |
| | YL}-METHYL-AMIDE | |
| 149 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 526[M+H]⁺ |
| | NAPHTHALEN-2-YL}-6,N- | |
| | DIMETHYL-NICOTINAMIDE | |
| 150 | N-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 529[M+H]⁺ |
| | NAPHTHALEN-2-YL}-2-FLUORO-N- | |
| | METHYL-BENZAMIDE | |
| 151 | FURAN-2-CARBOXYLIC ACID {6-[2- | Isolated in 99% purity @ |
| | (4-BENZO[D]ISOTHIAZOL-3-YL- | 214nm; LCMS(APCI) |
| | PIPERAZIN-1-YL)-ETHYL]-1,2,3,4- | 501[M+H]⁺ |
| | TETRAHYDRO-NAPHTHALEN-2- | |
| | YL}-METHYL-AMIDE | |
| 152 | CYCLOHEXANECARBOXYLIC | Isolated in 90% purity @ |
| | ACID {6-[2-(4- | 214nm; LCMS(APCI) |
| | BENZO[D]ISOTHIAZOL-3-YL- | 517[M+H]⁺ |
| | PIPERAZIN-1-YL)-ETHYL]-1,2,3,4- | |
| | TETRAHYDRO-NAPHTHALEN-2- | |
| | YL}-METHYL-AMIDE | |
| 153 | N-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 491[M+H]⁺ |
| | NAPHTHALEN-2-YL}-3,N- | |
| | DIMETHYL-BUTYRAMIDE | |
| 154 | N-{6-[2-(4-BENZO[D]ISOTHIAZOL-3- | Isolated in 95% purity @ |
| | YL-PIPERAZIN-1-YL)-ETHYL]- | 214nm; LCMS(APCI) |
| | 1,2,3,4-TETRAHYDRO- | 511[M+H]⁺ |
| | NAPHTHALEN-2-YL}-N-METHYL- | |
| | BENZAMIDE | |
| 155 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 517[M+H]⁺ |
| | NAPHTHALEN-2-YL}-2- | |
| | CYCLOPENTYL-N-METHYL- | |
| | ACETAMIDE | |
| 156 | N-{6-[2-(4-BENZO[D]ISOTHIAZOL-3- | Isolated in 95% purity @ |
| | YL-PIPERAZIN-1-YL)-ETHYL]- | 214nm; LCMS(APCI) |
| | 1,2,3,4-TETRAHYDRO- | 529[M+H]⁺ |
| | NAPHTHALEN-2-YL}-3-FLUORO-N- | |
| | METHYL-BENZAMIDE | |
| 157 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 479[M+H]⁺ |
| | NAPHTHALEN-2-YL}-2-METHOXY- | |
| | N-METHYL-ACETAMIDE | |
| 158 | N-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 477[M+H]⁺ |
| | NAPHTHALEN-2-YL}-N-METHYL- | |
| | BUTYRAMIDE | |
| 159 | N-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 477[M+H]⁺ |
| | NAPHTHALEN-2-YL}-N-METHYL- | |
| | ISOBUTYRAMIDE | |
| 160 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 505+H]⁺ |
| | NAPHTHALEN-2-YL}-3,3,N- | |
| | TRIMETHYL-BUTYRAMIDE | |
| 161 | N-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 491[M+H]⁺ |
| | NAPHTHALEN-2-YL}-2,2,N- | |
| | TRIMETHYL-PROPIONAMIDE | |
| 162 | PYRAZINE-2-CARBOXYLIC ACID | Isolated in 90% purity @ |
| | {6-[2-(4-BENZO[D]ISOTHIAZOL-3- | 214nm; LCMS(APCI) |
| | YL-PIPERAZIN-1-YL)-ETHYL]- | 513[M+H]⁺ |
| | 1,2,3,4-TETRAHYDRO- | |
| | NAPHTHALEN-2-YL}-METHYL- | |
| | AMIDE | |
| 163 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 523[M+H]⁺ |
| | NAPHTHALEN-2-YL}-2-(2- | |
| | METHOXY-ETHOXY)-N-METHYL- | |
| | ACETAMIDE | |
| 164 | N-{6-[2-(4-BENZO[D]ISOTHIAZOL-3- | Isolated in 99% purity @ |
| | YL-PIPERAZIN-1-YL)-ETHYL]- | 214nm; LCMS(APCI) |
| | 1,2,3,4-TETRAHYDRO- | 525[M+H]⁺ |
| | NAPHTHALEN-2-YL}-4,N- | |
| | DIMETHYL-BENZAMIDE | |
| 165 | 1-{6-[2-(4-BENZO[D]ISOTHIAZOL-3- | Isolated in 98% purity @ |
| | YL-PIPERAZIN-1-YL)-ETHYL]- | 214nm; LCMS(APCI) |
| | 1,2,3,4-TETRAHYDRO- | 492[M+H]⁺ |
| | NAPHTHALEN-2-YL}-1-METHYL-3- | |
| | PROPYL-UREA | |
| 166 | 1-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRHYDRO- | 506[M+H]⁺ |
| | NAPHTHALEN-2-YL}-3-BUTYL-1- | |
| | METHYL-UREA | |
| 167 | 1-{6-[2-(4-BENZO[D]ISOTHIAZOL-3- | Isolated in 98% purity @ |
| | YL-PIPERAZIN-1-YL)-ETHYL]- | 214nm; LCMS(APCI) |
| | 1,2,3,4-TETRAHYDRO- | 492[M+H]⁺ |
| | NAPHTHALEN-2-YL}-3- | |
| | ISOPROPYL-1-METHYL-UREA | |
| 168 | 1-{6-[2-(4-BENZO[D]ISOTHIAZOL-3- | Isolated in 100% purity @ |
| | YL-PIPERAZIN-1-YL)-ETHYL]- | 214nm; LCMS(APCI) |
| | 1,2,3,4-TETRAHYDRO- | 540[M+H]⁺ |
| | NAPHTHALEN-2-YL}-3-BENZYL-1- | |
| | METHYL-UREA | |
| 169 | N-{6-[2-(4-BENZO[D]ISOTHIAZOL-3- | Isolated in 90% purity @ |
| | YL-PIPERAZIN-1-YL)-ETHYL]- | 214nm; LCMS(APCI) |
| | 1,2,3,4-TETRAHYDRO- | 465[M+H]⁺ |
| | NAPHTHALEN-2-YL}-2-HYDROXY- | |
| | N-METHYL-ACETAMIDE | |

### Example 170

### ETHANESULFONIC ACID {6-[2-(4-BENZO[D]ISOTHIAZOL-3-YL-PIPERAZIN-1-YL)-ETHYL]-1,2,3,4-TETRAHYDRO-NAPHTHALEN-2-YL}-METHYL-AMIDE

6-[2-(4-Benzo[d]isothiazol-3-yl-piperazin-1-yl)-ethyl]-1,2,3,4-tetrahydro-naphthalen-2-ylamine was diluted to 0.10 M with anhydrous pyridine, then delivered to an 8 mL vial via pipette (0.10 mmol). Ethane sulfonyl chloride was diluted to 1.0 M with anhydrous pyridine then added to the amine solution (0.20 mmol), and the reaction was shaken overnight at room temperature. The following morning the reaction was diluted with 1 mL of anhydrous pyridine. PS-Trisamine scavenger resin was then added (0.20 mmol). The reaction was stirred for 3 h at room temperature. The resin was filtered and washed with one mL dichloromethane. The filtrate was concentrated via HT-12 GeneVac. Crude product was purified by HPLC (30×100 mm ODS-A C(18) 5u column). Ethanesulfonic acid {6-[2-(4-benzo[d]isothiazol-3-yl-piperazin-1-yl)-ethyl]-1,2,3,4-tetrahydro-naphthalen-2-yl}-methyl-amide was recovered in 97% purity @ 214 nm, LCMS (APCI) 499 [M+H]⁺.

Examples 171 and 172 were synthesized in combinatorial library format following the steps outlined in Example 170 on a 0.10 mmol scale using 6-[2-(4-Benzo[d]isothiazol-3-yl-piperazin-1-yl)-ethyl]-1,2,3,4-tetrahydro-naphthalen-2-ylamine with appropriate sulfonyl chloride. The crude products were purified by HPLC (30×100 mm ODS-A C (18) 5u column).

### Example 171

### PROPANE-1-SULFONIC ACID {6-[2-(4-BENZO[D]ISOTHIAZOL-3-YL-PIPERAZIN-1-YL)-ETHYL]-1,2,3,4-TETRAHYDRO-NAPHTHALEN-2-YL}-AMIDE

Isolated in 99% purity @ 214 nm; LCMS (APCI) 499 [M+H]⁺

### Example 172

### N-{6-[2-(4-BENZO[D]ISOTHIAZOL-3-YL-PIPERAZIN-1-YL)-ETHYL]-1,2,3,4-TETRAHYDRO-NAPHTHALEN-2-YL}-BENZENESULFONAMIDE

Isolated in 90% purity @ 214 nm; LCMS (APCI) 533 [M+H]⁺

1. A compound of the formula 1 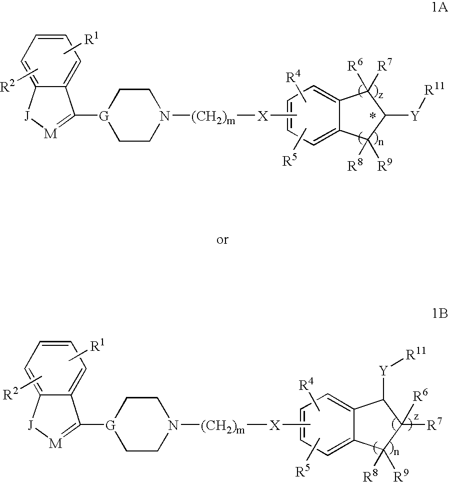
wherein J is S, SO, SO₂, CH₂, O, or NR¹⁰ wherein R¹⁰ is hydrogen, (C₁-C₆)alkyl, C(═O)(C₁-C₆)alkyl, or C(═O)O—(C₁-C₆)alkyl;
M is CH or N;
G is CH or N;
m is an integer from one to six;
X is O or NR³ wherein R³ is defined as R¹⁰ is defined above, C(═O), CHOH, CHOR³, CH(halo), or CHNR³R¹², wherein R¹² is defined as R¹⁰ is defined above;
or X is absent;
R¹ is hydrogen, halogen, cyano, (C₁-C₆)alkyl optionally substituted with from one to three fluorine atoms, (C₁-C₆)alkoxy optionally substituted with from one to three fluorine atoms, or R¹ forms a heterocyclic ring with R¹⁰;
R² is defined as R¹ with the proviso that R² cannot form a heterocyclic ring when R¹ is present;
R⁴ and R⁵ are, independently, hydrogen, halogen, cyano, amino-(C₁-C₆)alkyl, (C₁-C₆)alkylamino-(C₁-C₆)alkyl, di(C₁-C₆)alkylamino-(C₁-C₆)alkyl, hydroxy(C₁-C₆)alkyl, (C₁-C₆)alkoxy, or (C₁-C₆)alkoxyalkyl, wherein each of the alkoxy and alkyl moieties of the foregoing R⁴ and R⁵ groups can be optionally substituted with from one to three halo atoms, preferably with from one to three fluorine atoms;
R⁶, R⁷, R⁸, and R⁹ are, independently, hydrogen or (C₁-C₆)alkyl optionally substituted with from one to three fluorine atoms;
Y, when R¹¹ is present, is selected from O, NR¹³, wherein R¹³ is defined as R¹⁰ is defined above, or (CH₂)_{w} wherein w is an integer from one to six; or
Y, when R¹¹ is absent, is selected from (═O), hydroxy, NR¹³R¹⁴ wherein R¹³ and R¹⁴ are defined as R¹⁰ is defined above, and (CH₂)_{q}CH₃, wherein q is an integer from one to five;
n is an integer from one to three;
z is an integer from one to three; and
R¹¹ is hydrogen, (C₁-C₆)alkyl, —SO₂(C₁-C₆)alkyl, —SO²aryl, aryl, aryl-(C₁-C₆)alkyl, heteroaryl, heteroaryl-(C₁-C₆)alkyl, heterocyclyl, heterocyclyl-(C₁-C₄)alkyl, COR¹⁵, C(O)OR¹⁵, or C(O)NR¹⁵R¹⁶, wherein R¹⁵ and R¹⁶ are independently selected from (C₁-C₆)alkyl, aryl, heteroaryl, heteroaryl-(C₁-C₆)alkyl, aryl-(C₁-C₆)alkyl, heterocyclyl and heterocyclyl-(C₁-C₆)alkyl;
wherein said alkyl moieties within said R¹¹ groups can optionally be substituted with from one to three fluorine atoms, and the aryl, heteroaryl, and heterocyclyl moieties within said R¹¹ groups can optionally be substituted, with one or more substituents, preferably with from zero to two substituents, independently selected from (C₁-C₆)alkyl optionally substituted with from one to three fluorine atoms, (C₁-C₆)alkoxy optionally substituted with from one to three fluorine atoms, cyano, nitro, halo, amino, (C₁-C₆)alkylamino and di-(C₁-C₆)alkylamino; or
R¹¹ is absent;
with the proviso that the sum of n plus z cannot exceed 3;
and the pharmaceutically acceptable salts of such compounds.

2. A compound according to claim 1, wherein J is sulfur and M is nitrogen.

3. A compound according to claim 1, wherein G is N.

4. A compound according to claim 1, wherein m is two.

5. A compound according to claim 1, wherein X is absent.

6. A compound according to claim 1, wherein R¹ and R² are hydrogen.

7. A compound according to claim 1, wherein R⁴ and R⁵ are hydrogen.

8. A compound according to claim 1, wherein R⁶, R⁷, R⁸ and R⁹ are independently selected from hydrogen and methyl.

9. A compound according to claim 1, wherein YR¹¹ is NR¹³.

10. A compound according to claim 1, wherein Y is NR¹³R¹⁴.

11. A compound according to claim 1, wherein z is one.

12. A compound according to claim 1, wherein n is one.

13. A compound that is selected from the following compounds and their pharmaceutically acceptable salts:
N-{6-[2-(4-Benzo[d]isothiazol-3-yl-piperazin-1-yl)-ethyl]-indan-1-yl}-acetamide;
(S)(+)-N-{5-[2-(4-Benzo[d]isothiazol-3-yl-piperazin-1-yl)-ethyl]-indan-2-yl}-acetamide;
(R)(−)-N-{5-[2-(4-Benzo[d]isothiazol-3-yl-piperazin-1-yl)-ethyl]-indan-2-yl}-propionamide;
(S)(+)-N-{5-[2-(4-Benzo[d]isothiazol-3-yl-piperazin-1-yl)-ethyl]-indan-2-yl}-propionamide;
(S)(+)-Cyclopropanecarboxylic acid {5-[2-(4-Benzo[d]isothiazol-3-yl-piperazin-1-yl)-ethyl]-indan-2-yl}-amide;
(R)(−)-N-{5-[2-(4-Benzo[d]isothiazol-3-yl-piperazin-1-yl)-ethyl]-indan-2-yl}-N-methyl-acetamide;
(S)(+)-N-{5-[2-(4-Benzo[d]isothiazol-3-yl-piperazin-1-yl)-ethyl]-indan-2-yl}-N-methyl-acetamide;
{5-[2-(4-Benzo[d]isothiazol-3-yl-piperazin-1-yl)-ethyl]-1,1,3,3-tetramethyl-indan-2-yl}-methylamine;
N-{5-[2-(4-Benzo[d]isothiazol-3-yl-piperazin-1-yl)-ethyl]-1,1,3,3-tetramethyl-indan-2-yl}-N-methylacetamide;
N-{5-[2-(4-Benzo[d]isothiazol-3-yl-piperazin-1-yl)-ethyl]-1,1,3,3-tetramethyl-indan-2-yl}-acetamide;
N-{5-[2-(4-Benzo[d]isothiazol-3-yl-piperazin-1-yl)-ethyl]-1,1-dimethyl-indan-2-yl}-acetamide;
(+)-N-{5-[2-(4-Benzo[d]isothiazol-3-yl-piperazin-1-yl)-ethyl]-1,1-dimethyl-indan-2-yl}-acetamide;
(−)-N-{5-[2-(4-Benzo[d]isothiazol-3-yl-piperazin-1-yl)-ethyl]-1,1-dimethyl-indan-2-yl}-acetamide;
N-{6-[2-(4-Benzo[d]isothiazol-3-yl-piperazin-1-yl)-ethyl]-1,1-dimethyl-indan-2-yl}-acetamide;
(+)-N-{6-[2-(4-Benzo[d]isothiazol-3-yl-piperazin-1-yl)-ethyl]-1,1-dimethyl-indan-2-yl}-acetamide; and
(−)-N-{6-[2-(4-Benzo[d]isothiazol-3-yl-piperazin-1-yl)-ethyl]-1,1-dimethyl-indan-2-yl}-acetamide.

14. A pharmaceutical composition comprising a therapeutically effective amount of a compound according to claim 1 and a pharmaceutically acceptable carrier.

15. A method for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease, Huntington's disease, Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in the mammal, comprising administering to a mammal in need of such treatment an amount of a compound according to claim 1, or a pharmaceutically acceptable salt thereof, that is effective in treating such disorder or condition.

16. A method according to claim 15, wherein the disorder or condition that is being treated is selected from major depression, single episode depression, recurrent depression, child abuse induced depression, postpartum depression, dysthymia, cyclothymia and bipolar disorder.

17. A method according to claim 15, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

18. A method according to claim 15, wherein the disorder or condition that is being treated is selected from autism, pervasive development disorder, and attention deficit hyperactivity disorder.

19. A method according to claim 15, wherein the disorder or condition that is being treated is selected from generalized anxiety disorder, panic disorder, obsessive-compulsive disorder, post-traumatic stress disorder, and phobias, including social phobia, agoraphobia, and specific phobias.

20. A method according to claim 15, wherein the compound according to claim 1 is administered to a human for the treatment of any two or more comorbid disorders or conditions selected from those disorders and conditions referred to in any of the above methods.

21. A method according to claim 15, wherein the disorder or condition being treated is schizophrenia with concomitant depression.

22. A method according to claim 15, wherein the disorder or condition being treated is schizophrenia with concomitant anxiety.

23. A method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; attention deficit hyperactivity disorder (ADHD); behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to said mammal:
(a) a compound according to claim 1 or a pharmaceutically acceptable salt thereof; and
(b) another pharmaceutically active compound that is an antidepressant or an anti-anxiety agent, or a pharmaceutically acceptable salt thereof;
wherein the active agents “a” and “b” are present in amounts that render the combination effective in treating such disorder or condition.

24. A pharmaceutical composition comprising:
(a) a therapeutically effective amount of a compound according to claim 1;
(b) another pharmaceutically active agent that is an antidepressant or an anti-anxiety agent; and
(c) a pharmaceutically acceptable carrier.
